SEQUENCE LISTING (26 sequences)

SEQ ID NO: 1 - DNA, 38 bp, unidentified
  source 1..38: mol_type = unassigned DNA
ggcttgtcgacgacggcggactccgtcgtcaggatcat

SEQ ID NO: 2 - DNA, 48 bp, unidentified
  source 1..48: mol_type = unassigned DNA
ggtttgtctggtcaaccaccgcggactcagtggtgtacggtacaaacc

SEQ ID NO: 3 - DNA, 38 bp, unidentified
  source 1..38: mol_type = other DNA
ggcttgtcgacgacggcggtctccgtcgtcaggatcat

SEQ ID NO: 4 - DNA, 48 bp, unidentified
  source 1..48: mol_type = unassigned DNA
ggtttgtctggtcaaccaccgcggtctcagtggtgtacggtacaaacc

SEQ ID NO: 5 - DNA, 49 bp, unidentified
  source 1..49: mol_type = unassigned DNA
cggtgcgggtgccagggcgtgcccttggctccccgggcgcgtactccac

SEQ ID NO: 6 - DNA, 42 bp, unidentified
  source 1..42: mol_type = unassigned DNA
gtgccccaactggggtaacctttgagttctctcagttggggg

SEQ ID NO: 7 - protein, 500 aa, unidentified [sequence omitted]

SEQ ID NO: 8 - protein, 605 aa, unidentified
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 9 - DNA, 28 bp, unidentified
  source 1..28: mol_type = unassigned DNA
gcagagatccagtttggactagtctctc

SEQ ID NO: 10 - DNA, 21 bp, unidentified
  source 1..21: mol_type = unassigned DNA
gtgagcgaggaagcggaagag

SEQ ID NO: 11 - DNA, 28 bp, unidentified
  source 1..28: mol_type = unassigned DNA
atggtgagcaaaggtgaagaactgttca

SEQ ID NO: 12 - DNA, 31 bp, unidentified
  source 1..31: mol_type = unassigned DNA
atggcacaatctaagcatggattgactaagg

SEQ ID NO: 13 - DNA, 27 bp, unidentified
  source 1..27: mol_type = unassigned DNA
ctccagatccatcaaaaaaggctgtga

SEQ ID NO: 14 - DNA, 45 bp, unidentified
  source 1..45: mol_type = unassigned DNA
cttgtcagccatgatgtacacattatgactattgaagttatattc

SEQ ID NO: 15 - DNA, 17 bp, unidentified
  source 1..17: mol_type = unassigned DNA
acaccacgccacgttgc

SEQ ID NO: 16 - DNA, 19 bp, unidentified
  source 1..19: mol_type = unassigned DNA
ccgcaaagacagctccagc

SEQ ID NO: 17 - DNA, 73 bp, unidentified
  source 1..73: mol_type = unassigned DNA
agggagttttccacacggacacccccctcctcaccacagccctgccaggaccaccttatattcccaatggaag

SEQ ID NO: 18 - DNA, 19 bp, unidentified
  source 1..19: mol_type = unassigned DNA
ggaacggggctcagtctga

SEQ ID NO: 19 - DNA, 36 bp, unidentified
  source 1..36: mol_type = unassigned DNA
agttgtggtttgtccaaactcatcaatgtatcttat

SEQ ID NO: 20 - DNA, 19 bp, unidentified
  source 1..19: mol_type = unassigned DNA
ggaacggggctcagtctga

SEQ ID NO: 21 - DNA, 28 bp, unidentified
  source 1..28: mol_type = unassigned DNA
atggtgagcaaaggtgaagaactgttca

SEQ ID NO: 22 - DNA, 79 bp, unidentified
  source 1..79: mol_type = unassigned DNA
ttcctccgtgcgtcagttttacctgtgagataaggccagtagccagccccggcctaagttcaacgcgtataagatacat

SEQ ID NO: 23 - DNA, 24 bp, unidentified
  source 1..24: mol_type = unassigned DNA
ccctttcgctttcaagtccctgtt

SEQ ID NO: 24 - DNA, 19 bp, unidentified
  source 1..19: mol_type = unassigned DNA
ggcccactgtttccccttc

SEQ ID NO: 25 - DNA, 17 bp, unidentified
  source 1..17: mol_type = unassigned DNA
acaccacgccacgttgc

SEQ ID NO: 26 - DNA, 28 bp, unidentified
  source 1..28: mol_type = unassigned DNA
ctctggttctgggtacttttatctgtcc

(19) United States
(12) Patent Application Publication
(10) Pub. No.: US 2024/0352485 A1
(43) Pub. Date: Oct. 24, 2024

(54) COMPOSITIONS AND METHODS FOR THE PRODUCTION OF LIBRARIES

(71) Applicant: Flagship Pioneering Innovations VII, LLC, Cambridge, MA (US)

(72) Inventors: Patrick Leo JONES, Boston, MA (US); Adam Spier DINGENS, Cambridge, MA (US); Sukhmani Kaur BEDI, Arlington, MA (US); Markus Franz Friedrich ARNOLD, Watertown, MA (US)

(21) Appl. No.: 18/640,121

(22) Filed: Apr. 19, 2024

Related U.S. Application Data
(60) Provisional application No. 63/497,164, filed on Apr. 19, 2023.

(51) Int. Cl. C12N 15/867; C07K 14/015; C07K 14/155; C12N 15/864

(52) U.S. Cl. CPC C12N 15/867; C07K 14/015; C07K 14/155; C12N 15/8645; C12N 2740/10043; C12N 2740/15043; C12N 2750/14143; C12N 2800/30; C12N 2830/42; C12N 2840/203

(57) ABSTRACT

Provided herein are polynucleotides (e.g., plasmids), including transfer polynucleotides and landing pad polynucleotides, which are useful, e.g., in the generation of cell and virion libraries each expressing and encoding a protein of interest (e.g., viral entry proteins). The libraries described herein are further useful, e.g., in methods of assessing functional characteristics of the proteins of interest (e.g., neutralization of viral entry proteins by one or more antibodies).

### RELATED APPLICATIONS

This application claims priority to U.S. Ser. No. 63/497,164, filed Apr. 19, 2023, the entire contents of which is incorporated herein by reference.

### SEQUENCE LISTING

The instant application contains a Sequence Listing which has been submitted electronically in XML format and is hereby incorporated by reference in its entirety. Said XML copy, created on Apr. 17, 2024, is named 62801_59US01_SL.xml and is 28,672 bytes in size.

### 1. FIELD

This disclosure relates to compositions (e.g., polynucleotides, vectors, systems, cells) that are useful in, e.g., the generation of cell libraries encoding proteins of interest (e.g., viral entry proteins), that are further useful, e.g., in methods of efficiently assessing functional characteristics of the proteins of interest (e.g., viral entry proteins).

### 2. BACKGROUND

Pseudotyped viruses are engineered viruses comprising the structural and enzymatic core of one virus (e.g., a lentivirus) and the entry protein(s) of another virus. Various viruses can be utilized for the structural core of the pseudotyped virus, including, for example retroviruses (e.g., lentiviruses (such as HIV)) and MLV, and rhabdoviruses (such as VSV). Generally, one or more viral entry protein of interest is encoded in a vector (e.g., a plasmid) that is introduced into a producer cell line alongside helper plasmids containing viral packaging and enzymatic proteins. The viral particles produced are generally homogenous, expressing the viral entry protein(s) of interest on the surface of the particle and encoding the same viral entry protein(s) within the viral genome. Pseudotyped viruses can be utilized to assess such characteristics as e.g., virus entry and tropism.

### 3. SUMMARY

Provided herein are, inter alia, compositions (e.g., polynucleotides, vectors, systems, cells) that are useful in, e.g., the generation of cell libraries encoding proteins of interest (e.g., viral entry proteins), that are further useful, e.g., in methods of efficiently assessing functional characteristics of the proteins of interest (e.g., viral entry proteins).

Accordingly, in one aspect, provided herein are transfer polynucleotides comprising: a polynucleotide sequence encoding a protein of interest (e.g., a viral entry protein), one or more selectable marker genes, and a recombinase recognition site, wherein the transfer polynucleotide is transcriptionally inactive.

In some embodiments, the protein of interest comprises a barcode. In some embodiments, the transfer polynucleotide encodes more than one protein of interest. In some embodiments, the transfer polynucleotide encodes 1, 2, 3, 4, 5, or more proteins of interests.

In some embodiments, the transfer polynucleotide further comprises a partial viral genome. In some embodiments, the partial viral genome is a partial retrovirus genome, a partial lentivirus genome, or a partial adeno-associated virus (AAV) genome. In some embodiments, the partial viral genome comprises a long terminal repeat (LTR). In some embodiments, the partial viral genome comprises or consists of one LTR. In some embodiments, the LTR is a 3′ LTR. In some embodiments, the 3′ LTR comprises a U3 region. In some embodiments, the 3′ LTR does not contain a U3 region. In some embodiments, the 3′ LTR comprises a functional deletion of the U3 region. In some embodiments, the partial viral genome comprises a 3′ LTR and does not contain a 5′ LTR.

In some embodiments, the protein of interest is a viral entry protein (or a variant or fragment thereof). In some embodiments, the protein of interest is a naturally occurring viral entry protein, a naturally occurring viral entry protein variant (relative to a reference viral entry protein), a non-naturally occurring viral entry protein variant (relative to a reference viral entry protein), or a viral entry protein variant (relative to a reference viral entry protein) predicted to be naturally occurring at some point in time in the future. In some embodiments, the protein of interest is a viral entry protein from a circulating strain of a virus, from a seasonal strain of a virus, and/or from a pandemic strain of a virus. In some embodiments, the viral entry protein is a SARS-COV-2 spike protein. In some embodiments, the viral entry protein is an influenza HA protein. In some embodiments, the transfer polynucleotide encodes more than one viral entry protein. In some embodiments, the transfer polynucleotide encodes 1, 2, 3, 4, 5, or more viral entry proteins.

In some embodiments, the one or more selectable marker genes comprises an antibiotic resistance gene, a gene encoding a detectable protein, or a combination thereof. In some embodiments, the recombinase recognition site is a site that is recognized by a serine recombinase/integrase (e.g., Bxb1, φC31). In some embodiments, the recombinase recognition site is a site that is recognized by a Bxb1 recombinase. In some embodiments, the recombinase recognition site is an attB, attP, attP-GT, attP-GA, attB-GT, or attB-GA site.

In some embodiments, the method further comprises of one or more gene regulatory elements (e.g., all or a portion of one or more gene regulatory elements). In some embodiments, the one or more gene regulatory elements comprises an internal ribosome entry site (IRES), a polynucleotide sequence encoding a cleavable peptide (e.g., a 2A peptide), a viral posttranscriptional regulatory element (e.g., WPRE), a transcription termination sequence, and/or polyadenylation signal sequence (e.g., a polyA sequence), or any combination thereof. In some embodiments, the transfer polynucleotide does not contain a promoter.

In some embodiments, the transfer polynucleotide is isolated. In some embodiments, the transfer is integrated into a landing pad polynucleotide (e.g., a landing pad described herein) (e.g., a landing pad integrated into the genomic DNA of a cell). In some embodiments, the transfer polynucleotide is a DNA polynucleotide. In some embodiments, the transfer polynucleotide (e.g., DNA polynucleotide) is a plasmid.

In one aspect, provided herein are libraries (e.g., collection) of transfer polynucleotides (e.g., transfer plasmids) comprising a plurality of transfer polynucleotides described herein.

In some embodiments, the library comprises (a) a plurality of the transfer polynucleotides (e.g., plasmids) of the library comprise a polynucleotide encoding a different variant of a reference protein of interest (e.g., a reference viral entry protein), and optionally (b) a transfer polynucleotide encoding the reference protein of interest (e.g., a reference viral entry protein). In some embodiments, the reference protein is a reference viral entry protein (e.g., a viral entry protein described herein). In some embodiments, the transfer polynucleotides are plasmids.

In one aspect, provided herein are landing pad polynucleotides comprising: a partial viral genome, a recombinase recognition site, and a promoter operably linked to the recombinase recognition site.

In some embodiments, the partial viral genome comprises at least one LTR. In some embodiments, the partial viral genome comprises one or two LTRs. In some embodiments, the partial viral genome comprises a 5′LTR. In some embodiments, the partial viral genome comprises a 3′ LTR. In some embodiments, the partial viral genome comprises a 3′ LTR and a 5′ LTR. In some embodiments, the partial viral genome comprises a 5′ LTR and does not contain a 3′ LTR.

In some embodiments, the recombinase recognition site is a site that is recognized by a serine recombinase/integrase (e.g., Bxb1, φC31). In some embodiments, the recombinase recognition site is a site that is recognized by a Bxb1 recombinase. In some embodiments, the recombinase recognition site is an attB, attP, attP-GT, attP-GA, attB-GT, or attB-GA site.

In some embodiments, the promoter is a constitutive, inducible, and/or repressible promoter. In some embodiments, the promoter is an inducible and/or repressible promoter. In some embodiments, the landing pad polynucleotide further comprises one or more additional gene regulatory elements. In some embodiments, the one or more gene regulatory elements comprise a promoter, an enhancer, an internal ribosome entry site (IRES), a polynucleotide sequence encoding a cleavable peptide (e.g., a 2A peptide), a viral posttranscriptional regulatory element (e.g., WPRE), a transcription termination sequence, and/or polyadenylation signal sequence (e.g., a polyA sequence), or any combination thereof. In some embodiments, the landing pad polynucleotide further comprises a second promoter (e.g., a constitutive promoter).

In some embodiments, the landing pad polynucleotide further comprises one or more selectable marker genes. In some embodiments, the one or more selectable marker genes comprises an antibiotic resistance gene, a gene encoding a detectable protein, or a suicide gene, or a combination thereof.

In some embodiments, the landing pad polynucleotide further comprises a polynucleotide encoding a recombinase. In some embodiments, the recombinase is a serine recombinase/integrase (e.g., Bxb1, φC31). In some embodiments, the recombinase is a Bxb1 recombinase. In some embodiments, the polynucleotide encoding the recombinase is operably linked to a promoter. In some embodiments, the promoter is a constitutive promoter.

In some embodiments, the landing pad polynucleotide is isolated. In some embodiments, the landing pad is integrated into the genomic DNA of a cell. In some embodiments, the landing pad polynucleotide is a DNA polynucleotide. In some embodiments, the landing pad polynucleotide (e.g., DNA polynucleotide) is a plasmid.

In one aspect, provided herein are cells comprising a landing pad polynucleotide described herein integrated into the genomic DNA of the cell.

In some embodiments, the landing pad is integrated at a single genomic locus in the cell. In some embodiments, the landing pad is integrated at a single genomic locus in a single chromosome in the cell. In some embodiments, the single genomic locus is a safe harbor site (e.g., AAVS1, CCR5, Rosa26, or H11 (e.g., AAVS1)). In some embodiments, the cell comprises a single copy of the recombinase landing pad. In some embodiments, the cell is a human cell.

In one aspect, provided herein are libraries (e.g., collections) of cells comprising a plurality of cells comprising a landing pad polynucleotide described herein integrated into the genomic DNA of the cell and each cell further comprises a transfer polynucleotide (e.g., described herein) integrated into the integrated landing pad.

In some embodiments, each integrated transfer polynucleotide encodes a different protein of interest (e.g., a different viral entry protein). In some embodiments, the library comprises (a) a plurality of the integrated transfer polynucleotides each encodes a different variant of a reference protein of interest (e.g., a different variant of a reference viral entry protein), and optionally (b) a cell comprising an integrated transfer polynucleotide encoding the reference protein of interest (e.g., the reference viral entry protein). In some embodiments, each protein of interest encoded by each integrated transfer plasmid comprises a unique barcode.

In one aspect, provided herein are vectors comprising a transfer polynucleotide described herein. In some embodiments, the vector is a non-viral vector. In some embodiments, the vector is a plasmid.

In one aspect, provided herein are vectors comprising a landing pad polynucleotide described herein. In some embodiments, the vector is a non-viral vector. In some embodiments, the vector is a plasmid.

In one aspect, provided herein are cells (or population of cells) comprising any one or more of: a transfer polynucleotide described herein; a library of transfer polynucleotides described herein; a landing pad polynucleotide described herein; a cell library described herein; a vector described herein; or a system described herein.

In one aspect, provided herein are systems comprising (i) a transfer polynucleotide described herein; and (ii) a landing pad polynucleotide described herein.

In one aspect, provided herein are systems comprising (i) a transfer polynucleotide described herein; and (ii) a cell comprising a landing pad polynucleotide described herein integrated into the genomic DNA of the cell.

In one aspect, provided herein are systems comprising (i) a library of transfer polynucleotides described herein; and (ii) a cell comprising a landing pad polynucleotide described herein integrated into the genomic DNA of the cell.

In one aspect, provided herein are systems comprising (i) a cell library wherein each cell comprises a landing pad polynucleotide described herein integrated into the genomic DNA of the cell and a transfer polynucleotide (e.g., described herein) integrated into the integrated landing pad; and (ii) one or more helper plasmids encoding one or more viral proteins sufficient for virion production in combination with the library.

In one aspect, provided herein are systems comprising (i) a cell library made by a method described herein; and (ii) one or more of helper plasmids encoding one or more viral proteins sufficient for virion production in combination with the library.

In one aspect, provided herein are systems (i) a library of virions described herein; and (ii) a population of cells (e.g., human cells).

In one aspect, provided herein are compositions comprising any one or more of a transfer polynucleotide described herein; a library of transfer polynucleotides described herein; a landing pad polynucleotide described herein; a cell or population of cells described herein; a cell library described herein; a cell library made by a method described herein; a library of virions described herein; a vector described herein; or the system described herein; or any combination of any of the foregoing.

In one aspect, provided herein are kits comprising any one or more of a transfer polynucleotide described herein; a library of transfer polynucleotides described herein; a landing pad polynucleotide described herein; a cell or population of cells described herein; a cell library described herein; a cell library made by a method described herein; a library of virions described herein; a vector described herein; or the system described herein; or any combination of any of the foregoing.

In one aspect, provided herein are methods of making a cell library (e.g., collection), the method comprising: (a) making or obtaining a plurality of cells each comprising a landing pad polynucleotide described herein integrated into the genomic DNA of the cell; (b) introducing a library of transfer polynucleotides described herein into the cells; (c) culturing the cells under conditions and for a period of time sufficient to allow for recombinase mediated integration of a transfer polynucleotide into the integrated landing pad in a cell, wherein integration of a transfer polynucleotide into the landing pad enables transcription of: (i) the polynucleotide from the transfer polynucleotide encoding the protein of interest under the control of the promoter (e.g., inducible, repressible promoter) operably linked to the recombinase recognition site from the landing pad, and (ii) the one or more selectable marker genes from the transfer polynucleotide; (d) optionally selecting cells that comprise an integrated transfer polynucleotide by detecting expression of the one or more selectable marker genes from the transfer polynucleotide in the cells to thereby obtain a library of cells encoding proteins of interest.

In some embodiments, the recombinase recognition sites of the transfer polynucleotides and the landing pad polynucleotides are complementary.

In some embodiments, the transfer polynucleotide comprises a partial viral genome. In some embodiments, the partial viral genome of the transfer plasmid is complementary to the partial viral genome of the landing pad polynucleotide. In some embodiments, the partial virus genome of the landing pad comprises a 5′ LTR and the partial virus genome of the transfer polynucleotide comprises a 3′ LTR. In some embodiments, the partial virus genome of the landing pad comprises a 5′ LTR and a 3′ LTR.

In some embodiments, the recombinase is complementary to the recombinase recognition sites in the landing pad polynucleotide and the transfer polynucleotide. In some embodiments, the recombinase is introduced into the cells prior to, concurrently with, or subsequent to introduction of the transfer polynucleotides into the cells. In some embodiments, the landing pad comprises a polynucleotide sequence encoding the recombinase. In some embodiments, the recombinase is a Bxb1 recombinase. In some embodiments, the transfer polynucleotide comprises a Bxb1 attB site recombinase recognition site and the landing pad polynucleotide comprises a Bxb1 attP site.

In some embodiments, each different protein of interest (e.g., each different viral entry protein) comprises a unique barcode. In some embodiments, each protein of interest is a viral entry protein. In some embodiments, each protein is a different viral entry protein.

In some embodiments, the library of transfer polynucleotides comprises (a) a plurality of transfer polynucleotide, each encoding a different variants of a reference viral entry protein, and optionally (b) a transfer polynucleotide encoding the reference viral entry protein.

In some embodiments, the method further comprises transfecting the selected cells with one or more helper plasmids encoding one or more proteins viral proteins that enable formation of virus particles that express and encode the proteins (e.g., the viral entry proteins). In some embodiments, the helper plasmids encode one or more HIV-1 proteins selected from Tat, Gag-Pol, and Rev. In some embodiments, the method further comprises recovering, purifying, and/or quantifying the virions.

In one aspect, provided herein are libraries (e.g., collections) of cells made by a method described herein (e.g., a foregoing aspect).

In one aspect, provided herein are libraries (e.g., collections) of virions comprising a plurality of virions made a method described herein (e.g., a foregoing aspect).

In one aspect, provided herein are methods of making a library (e.g., collection) of virions, the method comprising (a) making or obtaining the library of cells wherein each cell of the library comprises a landing pad polynucleotide described herein integrated into the genomic DNA of the cell and a transfer polynucleotide (e.g., described herein) integrated into the integrated landing pad, and wherein each cell in the library comprises integrated transfer polynucleotide that encodes a different viral entry protein; (b) transfecting the library of cells of (a) with one or more helper plasmids encoding one or more viral proteins sufficient for virion production; and (c) culturing the cells under conditions and for sufficient time to allow for virion production; and (d) optionally isolating, purifying, and/or quantifying the produced virions.

In some embodiments, each cell in the library comprises integrated transfer polynucleotide that encodes a different viral entry protein.

In some embodiments, the cell library comprises (a) a plurality of cells each comprising an integrated transfer polynucleotide that encodes a different variant of a reference viral entry protein; and optionally (b) a cell comprising an integrated transfer polynucleotide encoding the reference viral entry protein.

In some embodiments, each virion in the library expresses (e.g., on the surface) and encodes a different viral entry protein. In some embodiments, the virion library comprises (a) plurality of virions each expressing on the surface and encoding a different variant of a reference viral entry protein; and optionally (b) a virion that expresses (e.g., on the surface) and encodes the reference viral entry protein. In some embodiments, each different viral entry protein comprises a unique barcode.

In some embodiments, the one or more helper plasmids encode one or more of the HIV gag, pol, RRE, and/or Rev proteins.

In one aspect, provided herein are libraries (e.g., a collection) of virions comprising a plurality of virions made by a method described herein (e.g., a foregoing aspect).

In one aspect, provided herein are methods of assessing the ability of one or more agents (e.g., antibodies) to neutralize a plurality of different viral entry proteins, the method comprising (a) making or obtaining the library of virions described herein (or made by a method described herein); (b) culturing a population of cells (e.g., a single population of cells) in the presence of the virion library of (a) and one or more agent (e.g., antibody) under conditions and for sufficient time to allow for infection of the cells; and (c) making a determination of whether the one or more agent (e.g., antibody) is capable of neutralizing a viral entry protein expressed by a virion of the library based on the ability of the virion within the library to infect the cells; wherein the one or more agent (e.g., antibody) is capable of neutralizing the viral entry protein if the virion does not infect the cells (or infection of the cells by the virion is not detectable).

In some embodiments, each virion in the library expresses (e.g., on the surface) and encodes a different viral entry protein. In some embodiments, the virion library comprises (a) a plurality of virions each encoding a different variant of a reference viral entry protein; and optionally (b) a virion encoding the reference viral entry protein. In some embodiments, each different viral entry protein comprises a unique barcode.

In some embodiments, the one or more agent is one or more antibody. In some embodiments, the one or more antibody is present in sera (or plasma) from a subject (e.g., a human subject, a non-human mammal subject) (or pooled sera (or plasma) from one or more subjects (e.g., human subjects, non-human mammal subjects)), wherein the sera (or plasma) is added to the cell culture. In some embodiments, the sera (or plasma) is obtained from a subject (or subjects) that are known to have been infected with or vaccinated against the virus that corresponds to the viral entry protein of the library. In some embodiments, the one or more antibody is a monoclonal antibody. In some embodiments, the one or more antibody is purified and isolated.

### 4. BRIEF DESCRIPTION OF THE FIGURES

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic showing an example approach of generating a cell library encoding virus packageable proteins (e.g., e.g., viral entry proteins) (e.g., uniquely barcoded viral entry proteins); as well as the virion library expressing (and encoding) the library of proteins (e.g., viral entry proteins) (e.g., uniquely barcoded viral entry proteins).

FIG. 2A is a schematic showing an example system and method of lentivirus pseudotyping, wherein a viral entry protein (VEP) is encoded in a plasmid that is introduced alongside helper plasmids containing viral packaging proteins. As there is only a single viral entry protein and a single genome to be packaged into the pseudotyped virus (e.g., replication-incompetent pseudotyped virus) across a pool of cells, the genotype-phenotype link is maintained. See, e.g., Duvergé, Alexis, and Matteo Negroni. “Pseudotyping Lentiviral Vectors: When the Clothes Make the Virus.” Viruses vol. 12,11 1311. 16 Nov. 2020, doi: 10.3390/v12111311, the entire contents of which are incorporated herein by reference for all purposes.

FIG. 2B is a schematic showing how the genotype-phenotype link in the exemplary lentivirus pseudotyping system described in FIG. 2A is lost when the system is utilized using a plurality of different plasmids, each encoding a different VEP. In this system, the introduction of a plurality of different plasmids into a cell line for packaging can result in multiple plasmids entering the same cell and the production of genotype-phenotype mismatched pseudotyped viruses—the VEP expressed on the surface of the virion does not match the barcoded genome within the virion (the genotype-phenotype link is broken).

FIG. 3A is a schematic showing an exemplary method described herein of generating a cell comprising an exemplary landing pad described herein. The landing pad (top right) is integrated into the genomic DNA of the cells (e.g., utilizing a CRISPR/Cas based method) (e.g., into a transcriptionally active safe harbor locus (e.g., AAVS1)). As shown in the figures, in preferred embodiments, the landing pad comprises one or more selectable marker gene (e.g., described herein) such that the cells with a successfully integrated landing pad can be selected. In some embodiments, the landing pad is only integrated into a single locus in a single chromosome (e.g., for master cell line creation).

FIG. 3B is a schematic showing an exemplary transfer plasmid library described herein (e.g., wherein the transfer plasmid library comprises at least one transfer plasmid comprising a polynucleotide encoding a reference viral entry protein (e.g., SARS-COV-2 spike) and a plurality of transfer plasmids each comprising a polynucleotide encoding a different variant of the reference viral entry protein (e.g., SARS-COV-2 spike), each being uniquely barcoded (left)). FIG. 3B further shows the generation of a cell library encoding barcoded viral entry proteins described herein (middle) through the introduction of the transfer plasmid library into a cell line with a stably integrated landing pad (e.g., as described in § 5.14.1). In preferred embodiments, the transfer plasmid comprises one or more selectable marker genes (e.g., described herein) (e.g., that are different from one or more selectable marker genes present in the integrated landing pad), such that cells with a successfully integrated transfer polynucleotide can be selected. FIG. 3B further shows the generation of a viral entry protein virion library described herein (see, e.g., § 5.14.2) through the introduction of one or more helper plasmids encoding the required viral proteins for virion production.

FIG. 4 is a schematic showing an exemplary method of utilizing a viral entry protein virion library described herein (see, e.g., § 5.7) to assess the ability of one or more antibodies (e.g., one or more recombinant monoclonal antibodies, antibodies present in sera from one or more subject (e.g., one or more human subject)) to neutralize the viral entry proteins expressed on the surface (and encoded within the genome of) the virions within the library. As shown on the left, the viral entry protein virion library can be utilized to infect cells in vitro with or without (control) sera (e.g., from one or more subject (e.g., human subject)) or one or more monoclonal antibody. Virions expressing viral entry proteins that are neutralized will not be capable of infecting the cells (middle-top); virions expressing select viral entry proteins that have escaped neutralization will be able to infect the cells (middle-middle); and in the control culture (without sera or monoclonal antibodies) all of the virions within the library will be able to infect the cells (middle-bottom). Where barcoded viral entry proteins are utilized, post-infection sequencing will detect only the viral entry proteins that enabled successful infection of the cells. The ratio of barcode present in the no sera (or no monoclonal antibody) control and the sera experimental group can be compared to identify e.g., relevant escape variations in the viral entry proteins (e.g., as compared to a reference viral entry protein).

FIG. 5 is a schematic showing the design of an exemplary recombinase landing pad described herein integrated into the AAVS1 genomic locus (top), an exemplary transfer plasmid of the disclosure (middle), and an exemplary recombined product of the disclosure (bottom) in which the transfer plasmid has been integrated into the recombinase landing pad by a Bxb1 recombinase. In this example, the recombinase landing pad includes a 5′ end of a lentiviral genome (5′ LTR and HIV-1 regulatory elements) and the transfer plasmid includes a 3′ end (3′ LTR) of a lentiviral genome. Bxb1 recombinase binds to the attP-GA and attB-GA sites on the landing pad and transfer plasmid, respectively, brings the sites together using protein-protein interactions, and carries out site-specific recombination (SSR) to generate two new sites, attL-GA and attR-GA, in the recombined product. Abbreviations for certain elements utilized in FIG. 5 are set forth below. 5′ LTR: 5′ long terminal repeat; PTRE3GS: doxycycline-inducible promoter, attP-GA: Bxb1 recombinase recognition site, BFP: blue fluorescent protein coding sequence selectable marker, Pcmv: cytomegalovirus constitutive promoter, BlastR: blasticidin resistance selectable marker; T2A: self-cleaving peptide coding sequence, BxB1: Bxb1 recombinase coding sequence, rtTa: element encoding tetR+VP16 fusion, AAVS1: adeno-associated virus integration site 1; attB-GA: Bxb1 recombinase recognition site, Viral Entry Protein: polynucleotide sequence encoding a viral entry protein of interest; 16x NBC: barcode sequence; WPRE: Woodchuck Hepatitis Virus Posttranscriptional Regulatory Element, 3′ LTR: 3′ long terminal repeat, bGH polyA: bovine growth hormone polyA element; IRES: internal ribosome entry site; PuroR: puromycin resistance selectable marker gene, ZsGreen: Zoanthus green fluorescent protein coding sequence.

FIG. 6 is a schematic showing the design of an exemplary recombinase landing pad described herein integrated into the AAVS1 genomic locus (top), an exemplary transfer plasmid (middle), and an exemplary recombined product (bottom) in which the transfer plasmid has been integrated into the recombinase landing pad by a Bxb1 recombinase. In this example, the recombinase landing pad includes two LTRs (5′ LTR and 3′ LTR) while the transfer plasmid lacks an LTR. Abbreviations of elements are same as for FIG. 5.

FIG. 7 is a schematic showing the design of an exemplary landing pad system described herein comprising a landing pad integrated into the AAVS1 genomic locus (top), two exemplary transfer plasmids (middle), and the recombined product post integration of the transfer plasmid into the landing pad (bottom). The components denoted by each abbreviation are set forth in Table 4 herein.

FIG. 8 is a schematic showing the design of an exemplary landing pad system described herein comprising two exemplary landing pads integrated into the AAVS1 genomic locus (top), an exemplary transfer plasmid (middle), and the recombined product post integration of the second (bottom) exemplary transfer plasmid into the landing pad (bottom). The components denoted by each abbreviation are set forth in Table 4 herein. The first (top) exemplary transfer plasmid comprises a viral 3′ LTR containing a U3 Region; and the second (bottom) exemplary transfer plasmid comprises a viral 3′ LTR lacking a U3 Region.

FIG. 9 is a schematic showing an exemplary landing pad plasmid (pLP) comprising a partial lentiviral genome, encoded integrase (e.g., Bxb1), encoded BFP, and an attP site; an exemplary transfer plasmid (pTF) comprising an encoded a viral entry protein (VEP) and an attB site; and the recombined product post integration of the landing pad into the subject DNA (e.g., genomic DNA in a cell) and integration of the transfer plasmid into the landing pad.

FIG. 10 is a schematic showing an exemplary method of assessing successful integration of a landing pad system described herein (as described in Example 3). Briefly, a landing pad plasmid and a transfer plasmid were transfected into target cells. After sufficient time and conditions to allow for integration of both the landing pad and the transfer polynucleotide, genomic DNA was extracted from the cells and analyzed by PCR utilizing primers designed for specific detection of the recombined product. Additional PCR analysis utilizing primers specific for the landing pad and the transfer polynucleotide was also conducted.

FIG. 11 is a schematic showing the positioning of primers pairs 1, 2, 3, and 4 for assessment of the integration of the landing pad system (as described in Example 3). Primer set 1 was designed to span the newly formed integration site at attR; primer set 2 was designed to span the newly formed integration site at attL, primer set 3 was designed to be specific for a portion of the landing pad; and primer set 4 was designed to be specific for a portion of the transfer polynucleotide.

FIG. 12 is an image of an electrophoresis gel showing the PCR products obtained utilizing primer set 1, 2, 3, or 4 in genomic DNA isolated from each of the treatment groups (landing pad plasmid control), transfer plasmid (control), and cells co-transfected with the landing pad plasmid and the transfer plasmid (as described in Example 3).

FIG. 13 is a schematic showing the generation of a stable HEK-293T landing pad cell line, wherein the landing pad polynucleotide is stably integrated into the cells utilizing a CRISPR/Cas based method and single cells in the transfected cultured are screened for landing pad integration (as described in Example 3).

FIG. 14 is a schematic showing the showing the positioning of primers pairs 6, 7, 8, and 9 for assessment of the integration of the landing pad system (as described in Example 3).

FIG. 15 is an image of an electrophoresis gel showing the PCR products obtained utilizing primer set 6, 7, 8, and 9 in genomic DNA isolated confirming stable integration of the landing pad into the cell (as described in Example 3).

FIG. 16 is a schematic showing the showing the generation of HEK-293T landing pad cells comprising an transfer polynucleotide into the landing pad, wherein the stable HEK-293T landing pad cells are transfected with a transfer plasmid and integration of the transfer polynucleotide into the cell and subsequently screened for integration (as described in Example 3).

FIG. 17 is an image of an electrophoresis gel showing the PCR products obtained utilizing primer set 1, 2, 3, and 4 in genomic DNA confirming stable integration of the transfer polynucleotide into the stable HEK-293T landing pad cell (as described in Example 3).

### 5. DETAILED DESCRIPTION

Viral particle libraries comprising a plurality of viruses pseudotyped with different viral entry proteins (e.g., SARS-COV-2 spike protein, Influenza hemagglutinin protein) are useful in numerous applications, including e.g., examining the effect of specific variations on viral entry and immune escape. As such, pseudotyped viral particle libraries are useful e.g., in the study of vaccines, anti-retrovirals, antibodies; e.g., particularly in the context of dangerous viruses. For any given viral entry protein there are countless numbers of variants (both naturally occurring and engineered). However, the current methods of generating such libraries are, inter alia, laborious, time consuming, and unscalable. As such, current libraries remain relatively small.

The inventors have, inter alia, designed methods of making, inter alia, large, consistent, and scalable viral entry protein pseudotyped virus libraries. As such, the compositions (e.g., transfer polynucleotides, landing pad polynucleotides, vectors, systems, cells, etc.) described herein are useful in, e.g., the generation of e.g., pseudotyped virus and cell libraries encoding viral entry proteins, that are further useful, e.g., in methods of efficiently assessing functional characteristics of the viral entry proteins. As such, the current disclosure provides compositions (e.g., transfer polynucleotides, landing pad polynucleotides, vectors, systems, cells, etc.); and their use in, inter alia, the generation of cell and virion based protein libraries.

TABLE OF CONTENTS

| | |
|---|---|
| 5.1 | Definitions |
| 5.2 | Transfer Polynucleotides (e.g., Transfer Plasmids) |
| 5.2.1 | Transfer Polynucleotide (e.g., Transfer Plasmid) Structure |
| 5.2.2 | Transcriptional Inactivity |
| 5.2.3 | Proteins of Interest |
| 5.2.3.1 | Viral Entry Proteins |
| 5.2.3.2 | Non-Viral Proteins |
| 5.2.4 | Recombinase Recognition Sites |
| 5.2.5 | Selectable Marker Genes |
| 5.2.6 | Gene Regulatory Elements |
| 5.2.7 | Partial Viral Genome |
| 5.2.8 | Integrated Transfer Polynucleotides |
| 5.3 | Libraries of Transfer Polynucleotides (e.g., Transfer Plasmids) |
| 5.4 | Landing Pad Polynucleotides (e.g., Landing Pad Plasmids) |
| 5.4.1 | Landing Pad Polynucleotide (e.g., Landing Pad |
| | Plasmid) Structure |
| 5.4.2 | Recombinases |
| 5.4.3 | Recombinase Recognition Sites |
| 5.4.4 | Partial Viral Genome |
| 5.4.5 | Selectable Marker Genes |
| 5.4.6 | Gene Regulatory Elements |
| 5.4.7 | Homology Arms for Site-Specific Integration |
| 5.4.8 | Integrated Landing Pads |
| 5.5 | Cells Comprising Integrated Landing Pads |
| 5.5.1 | Cell Types |
| 5.5.2 | Locus and Copy Number |
| 5.5.3 | Methods of Site Specific Landing Pad Integration |
| 5.6 | Cell Libraries (e.g., Encoding Viral Entry Proteins) |
| 5.7 | Virion Libraries |
| 5.8 | Polynucleotides |
| 5.9 | Vectors |
| 5.10 | Cells |
| 5.11 | Systems |
| 5.11.1 | Exemplary Systems |
| 5.11.2 | Complementary Elements |
| 5.11.3 | Additional Components |
| 5.11.3.1 | Recombinases |
| 5.11.3.2 | Helper Plasmids |
| 5.12 | Compositions |
| 5.13 | Kits |
| 5.14 | Methods |
| 5.14.1 | Methods of Making Cell Libraries (e.g., |
| | Encoding Viral Entry Proteins) |
| 5.14.2 | Methods of Making Virion Libraries |
| 5.14.3 | Methods of Utilizing Virion Libraries |

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided and vice versa.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

The term “barcode,” as used herein, generally refers to a label, or identifier, that conveys or is capable of conveying information about an analyte (e.g., a protein (e.g., the amino acid sequence of a protein), a polynucleotide (e.g., a polynucleotide encoding a protein) (e.g., the nucleotide sequence of a polynucleotide)). A barcode can be part of an analyte. A barcode can be independent of an analyte. A barcode can be a tag attached to an analyte (e.g., a protein, a polynucleotide (e.g., a polynucleotide encoding a protein)) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; peptide barcodes; random nucleotide and/or amino acid sequences; and synthetic nucleotide and/or amino acid sequences. A barcode can be attached to an analyte in a reversible or irreversible manner. In some embodiments, the barcodes described herein are irreversibly attached to an analyte. A barcode can be added to, for example, a polynucleotide (e.g., a DNA polynucleotide) (e.g., a polynucleotide encoding a protein), e.g., before, during, and/or after sequencing of the polynucleotide or an encoded protein. A barcode can be added to, for example, a protein, e.g., before, during, and/or after sequencing of the protein. Barcodes can allow for identification and/or quantification of individual sequencing-reads. Generally, different proteins will have unique barcodes to enable identification of each different protein. For example, barcoded variants of a reference will each have a unique barcode (relative to each other and the barcoded reference) to enable identification of each protein (each variant and the reference) through sequencing of the barcode.

The term “derived from” as used herein does not denote any specific process or method for obtaining the polynucleotide or protein. For example, the polynucleotide, or protein can be recombinant produced or chemically synthesized.

The “different” as used herein in reference to more than protein (e.g., viral entry proteins) means that the proteins do not have an identical amino acid sequence. This includes completely distinct proteins (e.g., SARS-Cov-2 spike protein and influenza A HA protein); and also includes a variant of a reference protein (e.g., a variant of a SARS-COV-2 spike protein and the reference SARS-COV-2 spike protein).

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein, the term “gene regulatory element” refers to an element (e.g., a polynucleotide sequence) that regulates expression of a gene. Gene regulatory elements are known in the art. Exemplary gene regulatory elements include, but art not limited to, e.g., promoters, enhancers, IRESs, 2A elements, termination elements, polyadenylation signals, etc. Gene regulatory elements can be derived from any suitable organism (e.g., human, viral, bacterial, etc.). Gene regulatory elements include those that are naturally occurring, variants of naturally occurring elements, or synthetic elements.

The terms “nucleic acid molecule,” “polynucleotide,” and “oligonucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleoside linkage, such as a phosphoroamidate linkage or a phosphorothioate linkage, instead of the phosphodiester found between the nucleotides of an unmodified nucleic acid molecule. Nucleic acid molecules can be linear or circular. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “operably linked” or “operably connected” refers to the linkage of two moieties in a functional relationship. For example, a gene regulatory element e.g., a promoter, enhancer, etc. is operably linked to a polynucleotide that encodes a protein if it affects the expression (e.g., transcription) of the polynucleotide that encodes the protein. For example, a polynucleotide element (e.g., gene, coding sequence (e.g., encoding a viral entry protein)) positioned in a polynucleotide construct (e.g., plasmid, recombinase landing pad, or recombined plasmid+recombinase landing pad) in such a way that enables expression of the polynucleotide element (e.g., gene, coding sequence (e.g., encoding a viral entry protein)) under control of a regulatory element (e.g., promoter) within the polynucleotide construct is operably linked to the regulatory element.

As used herein, the term “partial virus genome” or “partial viral genome” refers to a portion of a viral genome that comprises at least one long terminal repeat (LTR) of a virus (or a variant, fragment, and/or component thereof). This includes both naturally occurring LTRs (wild type and naturally occurring variants of a wild type LTR) and engineered variants thereof of naturally occurring LTRs. This includes both complete LTRs and incomplete LTRs (e.g., naturally occurring incomplete LTRs). Typical viral LTRs comprise a U3 region, an R region, and a U5 region. The LTR of a partial viral genome may contain a deletion of one or more component of an LTR (e.g., deletion of a U3 region) (e.g., as described herein).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where proteins are contemplated herein, it should be understood that proteins folded into their three-dimensional structure are also provided herein as well as polypeptides in the primary structure. Proteins can include more than one polypeptide (e.g., customarily referred to as the quaternary structure). As used herein, the term “promoter sequence operably linked to the recombinase recognition site”, means that a promoter sequence and recombinase recognition site are positioned in the recombinase landing pad such that integration of a polynucleotide (e.g., a transfer plasmid) of the disclosure at the recombinase recognition site in the landing pad will result in positioning of the polynucleotide sequence encoding a protein of interest near the promoter sequence, such that expression of the nucleotide sequence can occur under the control of the promoter sequence in the landing pad.

The terms, “recombinase” and “site-specific recombinase” are used interchangeably herein and refer to enzymes that can mediate rearrangements of DNA segments through the recognition of specific DNA sequences (recombination recognition sites). Site-specific recombinases are known in the art. The term includes, e.g., tyrosine site-specific recombinases (e.g., Cre, Dre, Flp, KD, B2, B3); tyrosine integrases (e.g., λ, HK022, HP01); serine resolvases/invertases (e.g., γδ, ParA, Tn3, Gin); and serine integrases (e.g., φC31, Bxb1, and R4). In some embodiments, the recombinase is a serine integrase. In specific embodiments, the recombinase is Bxb1.

As used herein, the term “recombinase recognition site” or “recombinase attachment site” refers to a polynucleotide sequence that is recognized by a site-specific recombinase.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleoside linkages.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments the subject is a non-human animal. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Palcaognathac (e.g., ostriches, emus), Columbiformes (e.g., pigeons, doves), or Psittaciformes (e.g., parrots). In some embodiments, the subject is a ferret, hamster, mouse, or non-human primate. In some embodiments, the subject is a ferret.

As used herein, the term “U3 Region” in reference to a viral 3′ LTR refers to the region of the viral 3′ LTR that comprises the promoter and/or sequences that drive viral transcription.

As used herein, the term “variant” or “variation” with reference to a polynucleotide, refers to a polynucleotide that comprises at least one substitution, alteration, inversion, addition, or deletion of nucleotide compared to a reference polynucleotide. As used herein, the term “variant” or “variation” with reference to a protein refers to a protein that comprises at least one substitution, alteration, inversion, addition, or deletion of an amino acid residue compared to a reference protein.

As used herein, the terms “variant protein” or “variant of a reference protein” and the like refer to a protein comprising at least one amino acid variation relative to the amino acid sequence of a reference protein. For example, a variant of a reference protein can differ from a reference protein by the addition, deletion, or substitution (or any combination thereof) of 1, 2, 3, 4, 5, 6, 7, 8, 9, 10, 11, 12, 13 14, 15, 16, 17, 18, 19, 20, 50, or 100 or more amino acid residues. In some embodiments, the variant differs from a reference protein by the addition, deletion, or substitution (or any combination thereof) of about 1, 2, 3, 4, 5, 10, 15, 20, 50, 100, or more amino acid residues.

As used herein, the term “viral entry protein” or “virus entry protein” refers to a viral protein (or any naturally occurring variants, engineered variants thereof, and/or variants predicted to naturally occurring at some point in the future) that functions (at least in part) to mediate entry of the virus into a host cell. The viral entry protein can be from any enveloped virus. Entry proteins of enveloped viruses are typically exposed on the surface of the envelope. Entry of enveloped viruses into cells is typically mediated (at least in part) through the fusion of the viral and cell membrane. In some cases, a single viral entry protein is sufficient to facilitate entry. In some cases, a single viral entry protein is not sufficient to facilitate entry and a plurality of viral protein components are required.

### 5.2 Transfer Polynucleotides (e.g., Transfer Plasmids)

Provided herein are, inter alia, transfer polynucleotides (e.g., transfer plasmids) that encode a protein of interest (e.g., a viral entry protein) (e.g., that are transcriptionally inactive). The transfer polynucleotides can take any suitable form of a polynucleotide (e.g., described herein, see, e.g., § 5.8) or a polynucleotide incorporated in a vector (e.g., a plasmid) (e.g., a vector described herein, see, e.g., § 5.9).

In some embodiments, the transfer polynucleotide is double stranded. In some embodiments, the transfer polynucleotide is single stranded. In some embodiments, the transfer polynucleotide is linear. In some embodiments, the transfer polynucleotide is circular. In some preferred embodiments, the transfer polynucleotide (e.g., transfer plasmid) is circular. The transfer polynucleotide can comprise DNA nucleotides and/or RNA nucleotides. In some embodiments, the transfer polynucleotide comprises one or more non-naturally occurring nucleotide. In some preferred embodiments, the transfer polynucleotide is a DNA polynucleotide. In some preferred embodiments, the transfer polynucleotide (e.g., transfer plasmid) is a circular double-stranded DNA molecule. In some embodiments, the transfer polynucleotide (e.g., transfer plasmid) is a circular single-stranded DNA molecule.

In some embodiments, the transfer polynucleotide is incorporated into a vector (i.e., a transfer vector) (e.g., a vector described herein, see, e.g., § 5.9). In some embodiments, the vector is a plasmid (i.e., a transfer plasmid). In some embodiments, the vector is a viral vector (i.e., a transfer viral vector). Suitable vectors (e.g., gene delivery vectors, plasmids, viral vectors, and non-viral vectors) for use in preparing transfer polynucleotides of the disclosure are known in the art and are commercially available. Exemplary suitable vectors are also described herein, see, e.g., § 5.9.

### 5.2.1 Transfer Polynucleotide (e.g., Transfer Plasmid) Structure

In some embodiments, the transfer polynucleotide (e.g., transfer plasmid) comprises (i) a polynucleotide encoding one or more protein of interest (e.g., a viral entry protein of interest (see, e.g., § 5.2.3.1)); and one or more of (ii) a recombinase recognition site (see, e.g., § 5.2.4); (iii) one or more selectable marker genes (see, e.g., § 5.2.5); (iv) one or more gene regulatory elements (see, e.g., § 5.2.6)); and/or (v) a partial viral genome (see, e.g., § 5.2.7).

In some embodiments, the transfer polynucleotide (e.g., transfer plasmid) comprises (i) a polynucleotide encoding one or more protein of interest (e.g., a viral entry protein of interest (see, e.g., § 5.2.3.1)); and one or more of (ii) one or more selectable marker genes (see, e.g., § 5.2.5); (ii) a recombinase recognition site (see, e.g., § 5.2.4); and/or (iii) a partial viral genome (see, e.g., § 5.2.7).

In some embodiments, the transfer polynucleotide (e.g., transfer plasmid) comprises (i) polynucleotide encoding one or more protein of interest (e.g., a viral entry protein of interest (see, e.g., § 5.2.3.1)) and (ii) a recombinase recognition site (see, e.g., § 5.2.4); and one or more of (iii) one or more selectable marker genes (see, e.g., § 5.2.5); (iv) one or more gene regulatory elements (see, e.g., § 5.2.6)); and/or (v) a partial viral genome (see, e.g., § 5.2.7).

In some embodiments, the transfer polynucleotide (e.g., transfer plasmid) comprises (i) a polynucleotide encoding one or more protein of interest (e.g., a viral entry protein of interest (see, e.g., § 5.2.3.1)); and one or more of (ii) a recombinase recognition site (see, e.g., § 5.2.4); (iii) a polynucleotide sequence encoding one or more selectable marker genes (see, e.g., § 5.2.5); and/or (iv) one or more gene regulatory elements (see, e.g., § 5.2.6)).

In some embodiments, the transfer polynucleotide (e.g., transfer plasmid) comprises (i) a polynucleotide encoding one or more protein of interest (e.g., a viral entry protein of interest (see, e.g., § 5.2.3.1)) and (ii) a recombinase recognition site (see, e.g., § 5.2.4); and one or more of (iii) one or more selectable marker genes (see, e.g., § 5.2.5); and/or (iv) one or more gene regulatory elements (see, e.g., § 5.2.6)).

In some embodiments, the transfer polynucleotide (e.g., transfer plasmid) comprises (i) a polynucleotide encoding one or more protein of interest (e.g., a viral entry protein of interest (see, e.g., § 5.2.3.1)); and (ii) a recombinase recognition site (see, e.g., § 5.2.4).

In some embodiments, the transfer polynucleotide (e.g., transfer plasmid) comprises (i) a polynucleotide encoding one or more protein of interest (e.g., a viral entry protein of interest (see, e.g., § 5.2.3.1)); (ii) a recombinase recognition site (see, e.g., § 5.2.4); and (iii) one or more selectable marker genes (see, e.g., § 5.2.5).

In some embodiments, the transfer polynucleotide (e.g., transfer plasmid) comprises (i) a polynucleotide encoding one or more protein of interest (e.g., a viral entry protein of interest (see, e.g., § 5.2.3.1)); (ii) a recombinase recognition site (see, e.g., § 5.2.4); (iii) one or more selectable marker genes (see, e.g., § 5.2.5); and (iv) one or more gene regulatory elements (see, e.g., § 5.2.6)).

In some embodiments, the transfer polynucleotide (e.g., transfer plasmid) comprises (i) a polynucleotide encoding one or more protein of interest (e.g., a viral entry protein of interest (see, e.g., § 5.2.3.1)); (ii) a recombinase recognition site (see, e.g., § 5.2.4); (iii) one or more selectable marker genes (see, e.g., § 5.2.5); (iv) one or more gene regulatory elements (see, e.g., § 5.2.6)); and (v) a partial viral genome (see, e.g., § 5.2.7).

### 5.2.2 Transcriptional Inactivity

In preferred embodiments, the transfer polynucleotides (e.g., transfer plasmids) described herein are transcriptionally inactive. Transcriptionally inactive transfer polynucleotides (e.g., transfer plasmids) are not capable of being transcribed unless they are e.g., integrated into the genome of a cell. For example, transcriptionally inactive transfer polynucleotides (e.g., transfer plasmids) described herein lack one or more gene regulatory elements (e.g., promoters, enhancers and/or other elements) necessary to direct transcription of the transfer polynucleotide (e.g., or at least a portion of the transfer polynucleotide (e.g., one or more protein coding region)). Accordingly, transcriptionally inactive transfer polynucleotides (e.g., transfer plasmids) that have been introduced into a cell, but which have not been integrated into the cell's DNA (e.g., into a landing pad described herein), will not be transcribed. Upon integration of the transfer polynucleotide (e.g., transfer plasmid) into the cell's genome (for example, into a landing pad described herein that has been integrated into the cell's genome) transcription of the transfer polynucleotide (e.g., or at least a portion of the transfer polynucleotide (e.g., one or more protein coding region)) can occur under the control of gene regulatory sequences (e.g., one or more inducible or constitutive promoters) in the cell's endogenous genomic DNA, and/or in the integrated landing pad DNA (e.g., gene regulatory elements in a landing pad described herein integrated in the cell's genome) (as discussed further herein).

### 5.2.3 Proteins of Interest

The transfer polynucleotides (e.g., transfer plasmids) described herein comprise a polynucleotide sequence encoding one or more protein of interest. The polynucleotide sequence encoding the protein(s) of interest can be, e.g., a gene sequence (e.g., comprising one or more exons; comprising one or more introns and one or more exons; and/or other gene regulatory elements); or a coding sequence (e.g., an open reading frame sequence).

The protein of interest can be any peptide or protein (e.g., an enzyme, a structural protein, a targeting protein, a signaling protein, an antibody (or antigen-binding fragment thereof), an viral envelope protein, a viral fusion protein, etc.). In some embodiments, the protein of interest is a non-viral protein. In preferred embodiments, the protein of interest is a viral protein (e.g., a viral entry protein) (see, e.g., § 5.2.3.1). In most preferred embodiments, the protein of interest is a viral entry protein (see, e.g., § 5.2.3.1).

The protein of interest can be a reference protein (e.g., a naturally-occurring wildtype protein), or a variant of a reference protein. Variants include, e.g., naturally occurring variants and engineered (non-naturally occurring) variants.

In some embodiments, the protein of interest comprises one or more heterologous sequence or tag. In some embodiments, the protein of interest comprises a purification sequence or tag. Examples of suitable purification tags are known to those of skill in the art. In some embodiments, the protein of interest comprises a detectable tag. In some embodiments, the protein of interest comprises a unique detectable tag. In some embodiments, the protein of interest comprises a barcode sequence or tag (e.g., a unique barcode sequence or tag) (e.g., to facilitate sequencing and identification of the protein). In some embodiments, the protein of interest comprises a barcode sequence (e.g., a unique barcode sequence or tag) (e.g., to facilitate sequencing and identification of the protein). Examples of suitable barcode sequences/tags for use in the transfer polynucleotides described herein are known to those of skill in the art.

In some embodiments, the transfer polynucleotide encodes 1 protein of interest. In some embodiments, the transfer polynucleotide encodes more than one protein of interest. In some embodiments, the transfer polynucleotide encodes 1, 2, 3, 4, 5 or more proteins of interest. In some embodiments, the transfer polynucleotide encodes 1, 2, 3, or 4 proteins of interest. In some embodiments, the transfer polynucleotide encodes 1 protein of interest. In some embodiments, the transfer polynucleotide encodes 2 proteins of interest. In some embodiments, the transfer polynucleotide encodes 3 proteins of interest. In some embodiments, the transfer polynucleotide encodes 4 proteins of interest. In some embodiments, the transfer polynucleotide encodes 5 proteins of interest. In some embodiments, the transfer polynucleotide encodes 1 protein of interest and one or more other proteins.

### 5.2.3.1 Viral Entry Proteins

In preferred embodiments, the protein of interest is a viral protein. The viral entry protein can be any viral entry protein from any enveloped virus. Viral entry proteins include, e.g., naturally occurring proteins, naturally occurring variants, non-naturally occurring variants, and variants predicted to be naturally occurring at some future time point.

In some embodiments, the viral entry protein is a reference viral entry protein (e.g., a naturally occurring wildtype protein). In some embodiments, the viral entry protein is a variant of a reference viral entry protein. In some embodiments, the viral entry protein is a naturally occurring variant of a reference viral entry protein. In some embodiments, the viral entry protein is a non-naturally occurring variant of a reference viral entry protein. In some embodiments, the viral entry protein is a variant of a reference viral entry protein that is predicted to be naturally occurring at some point in time in the future.

In some embodiments, the viral entry protein is from a circulating strain of a virus. In some embodiments, the viral entry protein is from a seasonal strain of a virus. In some embodiments, the viral entry protein is from a pandemic strain of a virus.

Exemplary E enveloped viruses and corresponding viral entry proteins are set forth in Table 1. The viruses and entry proteins set forth in Table 1 are exemplary only and not intended to be limiting in any way.

TABLE 1
Exemplary Enveloped Viruses and Entry Proteins.

| | Exemplary | Exemplary Viral |
|---|---|---|
| Virus family | Representatives | Entry Proteins |
| Coronaviridae | SARS-CoV-2 | Spike protein |
| | SARS-CoV | Spike protein |
| | MERS-CoV | Spike protein |
| Orthomyxoviridae | Influenza A virus | Hemagglutinin (HA) |
| | Influenza B virus | HA |
| Retroviridae | HIV-1 | Envelope glycoprotein |
| | HIV-2 | Envelope glycoprotein |
| Filoviridae | Ebola virus | GP glycoprotein |
| Paramyxoviridae | Measles | G glycoprotein/ |
| | | F glycoprotein |
| | Mumps | HN glycoprotein/ |
| | | F glycoprotein |
| | Respiratory | G glycoprotein/ |
| | Syncytial virus | F glycoprotein |
| | Parainfluenza | G glycoprotein |
| | Sendai virus | F glycoprotein |
| Flaviviridae | Dengue virus | E protein |
| | Yellow Fever virus | E protein |
| | West Nile virus | E protein |
| | Zika virus | E protein |
| | Japanese encephalitis virus | E protein |
| Alphaviridae | Semliki Forest virus | E1 glycoprotein/ |
| | | E2 glycoprotein |
| Rhabdoviridae | Vesicular stomatitis virus | G glycoprotein |
| Baculoviridae | Baculovirus | Gp64 glycoprotein |
| Arenaviridae | Lassa virus | GP1, GP2, SSP |

In some embodiments, the viral entry protein is from a circulating strain of a virus from a virus family listed in Table 1; from a virus set forth in Table 1; or a viral entry protein listed in Table 1. In some embodiments, the viral entry protein is from a seasonal strain of a virus from a virus family listed in Table 1; from a virus set forth in Table 1; or a viral entry protein listed in Table 1. In some embodiments, the viral entry protein is from a pandemic strain of a virus from a virus family listed in Table 1; from a virus set forth in Table 1; or a viral entry protein listed in Table 1.

In some embodiments, the viral entry protein is from a family listed in Table 1. In some embodiments, the viral entry protein is from a virus listed in Table 1. In some embodiments, the viral entry protein is listed in Table 1.

In some embodiments, the viral entry protein is a SARS-COV-2 spike protein. In some embodiments, the viral entry protein is a SARS-COV spike protein. In some embodiments, the viral entry protein is a MERS-COV spike protein. In some embodiments, the viral entry protein is an Influenza virus HA protein. In some embodiments, the viral entry protein is an Influenza A virus HA protein. In some embodiments, the viral entry protein is an Influenza B virus HA protein. In some embodiments, the viral entry protein is a HIV gp41 protein. In some embodiments, the viral entry protein is a HIV-1 gp41 protein. In some embodiments, the viral entry protein is a HIV-2 gp41 protein. In some embodiments, the viral entry protein is an Ebola virus GP protein. In some embodiments, the viral entry protein is a Sendai virus F protein. In some embodiments, the viral entry protein is a Semliki Forest virus El protein. In some embodiments, the viral entry protein is a Dengue virus E protein. In some embodiments, the viral entry protein is a Vesicular stomatitis virus G protein. In some embodiments, the viral entry protein is a Baculovirus GP64 protein. In some embodiments, the viral entry protein is a measles G glycoprotein. In some embodiments, the viral entry protein is a measles F glycoprotein. In some embodiments, the viral entry protein is a measles G glycoprotein and F glycoprotein. In some embodiments, the viral entry protein is a mumps HN glycoprotein. In some embodiments, the viral entry protein is a mumps HN and F glycoprotein. In some embodiments, the viral entry protein is a mumps F glycoprotein. In some embodiments, the viral entry protein is a respiratory syncytial virus G glycoprotein. In some embodiments, the viral entry protein is a parainfluenza G glycoprotein. In some embodiments, the viral entry protein is a parainfluenza F glycoprotein. In some embodiments, the viral entry protein is a parainfluenza G glycoprotein and F glycoprotein. In some embodiments, the viral entry protein is a dengue virus E protein. In some embodiments, the viral entry protein is a yellow fever virus E protein. In some embodiments, the viral entry protein is a West Nile virus E protein. In some embodiments, the viral entry protein is a zika virus E protein. In some embodiments, the viral entry protein is a Japanese encephalitis virus E protein. In some embodiments, the viral entry protein is a Lassa virus GP1 protein. In some embodiments, the viral entry protein is a Lassa virus GP2 protein. In some embodiments, the viral entry protein is a Lassa virus SSP protein. In some embodiments, the viral entry protein is a Lassa virus GP1, GP2, and SSP protein. In some embodiments, the viral entry protein is a reference viral entry protein (e.g., a naturally occurring wildtype protein) listed in Table 1. In some embodiments, the viral entry protein is a variant of a reference viral entry protein listed in Table 1. In some embodiments, the viral entry protein is a naturally occurring variant of a reference viral entry protein listed in Table 1. In some embodiments, the viral entry protein is a non-naturally occurring variant of a reference viral entry protein listed in Table 1. In some embodiments, the viral entry protein is a variant of a reference viral entry protein listed in Table 1 that is predicted to be naturally occurring at some point in time in the future.

In some embodiments, the viral entry protein is a reference SARS-COV-2 spike protein (e.g., a naturally occurring wildtype SARS-COV-2 spike protein). In some embodiments, the viral entry protein is a variant of a reference SARS-COV-2 spike protein. In some embodiments, the viral entry protein is a naturally occurring variant of a reference SARS-COV-2 spike protein. In some embodiments, the viral entry protein is a non-naturally occurring variant of a reference SARS-COV-2 spike protein. In some embodiments, the viral entry protein is a variant of a reference SARS-COV-2 spike protein that is predicted to be naturally occurring at some point in time in the future.

In some embodiments, the viral entry protein is a reference Influenza HA protein (e.g., a naturally occurring wildtype Influenza HA protein). In some embodiments, the viral entry protein is a variant of a reference Influenza HA protein. In some embodiments, the viral entry protein is a naturally occurring variant of a reference Influenza HA protein. In some embodiments, the viral entry protein is a non-naturally occurring variant of a reference Influenza HA protein. In some embodiments, the viral entry protein is a variant of a reference Influenza HA protein that is predicted to be naturally occurring at some point in time in the future.

In some embodiments, the transfer polynucleotide encodes 1 viral entry protein. In some embodiments, the transfer polynucleotide encodes more than one viral entry protein. In some embodiments, the transfer polynucleotide encodes 1, 2, 3, 4, 5 or more viral entry proteins. In some embodiments, the transfer polynucleotide encodes 1, 2, 3, or 4 viral entry proteins. In some embodiments, the transfer polynucleotide encodes 1 viral entry protein. In some embodiments, the transfer polynucleotide encodes 2 viral entry proteins. In some embodiments, the transfer polynucleotide encodes 3 viral entry proteins. In some embodiments, the transfer polynucleotide encodes 4 viral entry proteins. In some embodiments, the transfer polynucleotide encodes 5 viral entry proteins. In some embodiments, the transfer polynucleotide encodes 1 viral entry proteins and one or more other proteins.

For example, some viruses require more than one viral entry protein to mediate entry into a cell (see, e.g., Table 1 (e.g., measles, mumps, respiratory syncytial virus, Semliki forest virus, Lassa virus). As such, in some embodiments, the transfer polynucleotide encodes each of the viral entry proteins.

### 5.2.3.2 Non-Viral Proteins

In some embodiments, the encoded protein of interest is a non-viral protein. The reference protein can be any peptide or protein (e.g., an enzyme, a structural protein, a targeting protein or peptide, a signaling protein, an antibody or antigen-binding fragment of an antibody). In some embodiments, the reference protein is a non-viral protein (e.g., a cell targeting protein or peptide, such as a single-chain variable fragment (scFv) or Fab fragment of an antibody). In some embodiments, the protein of interest is an antibody (or a functional fragment or variant thereof).

### 5.2.4 Recombinase Recognition Sites

As described above, in preferred embodiments, the transfer polynucleotides (e.g., transfer plasmids) described herein comprise a recombinase recognition site (also known and referred to herein as a recombinase attachment (att) site).

As described herein, site specific recombinases are enzymes that can mediate rearrangements of DNA segments through the recognition of specific DNA sequences (recombination recognition sites). Site-specific recombinases and their cognate recognition sites are known in the art. For example, exemplary site-specific recombinases, include, but are not limited to serine recombinases, or serine integrases, which are also known as resolvases (e.g., Bxb1 recombinase/integrase, φC31 integrase, γδ resolvase, and Gin invertase); and tyrosine recombinases (e.g., Cre, Flp, and λ integrase).

Exemplary site-specific tyrosine and serine recombinases and their recognition sites are described, for example, in Gaj T. et al, Expanding the scope of site-specific recombinases for genetic and metabolic engineering. Biotechnol Bioeng. 2014 January; 111 (1): 1-15, doi: 10.1002/bit.25096. Epub 2013 Sep. 13 (see, e.g., Table 1 on page 29) (herein after “Gaj 2014”), Durrant, M. G., et al., Systematic discovery of recombinases for efficient integration of large DNA sequences into the human genome. Nat Biotechnol 41, 488-499 (2023) (see, e.g., Suppl. Table 2) (hereinafter “Durrant 2023”), and Merrick, C. A., et al., Serine Integrases: Advancing Synthetic Biology. ACS Synth. Biol. 2018, 7, 299-310 (hereinafter “Merrick 2018”), the entire contents of each of which are incorporated herein by reference for all purposes. Additional examples of Bxb1 recognition sites include the attP-GT and attP-GA sites described in Low, B., et al., Scientific Reports (2022) 12:5424 (hereinafter “Low 2022”), the entire contents of which are incorporated herein by reference for all purposes. Additional Bxb1 recognition sites are described in, e.g., Zhang, Q., Azarin, S. M. & Sarkar, C. A. Model-guided engineering of DNA sequences with predictable site-specific recombination rates. Nat Commun 13, 4152 (2022) (hereinafter “Zhang 2022”); https://doi.org/10.1038/s41467-022-31538-3, the entire contents of which are incorporated herein by reference for all purposes.

Exemplary recognition sites for Bxb1 recombinase/integrase and φC31 integrase are set forth in Table 2.

TABLE 2
Recognition Sites for Bxb1 Recombinase.

| Description | | Nucleotide Sequence of Attachment Sites | SEQ |
|---|---|---|---|
| Recombinase | Attachment Sites | (5′ to 3′) | ID NO |
| Bxb1 | Attachment Bacterial | GGCTTGTCGACGACGGCGGACTCCGTCGTCAGG | 1 |
| | (attB) Site (attB-GA) | ATCAT | |
| | Attachment Phage | GGTTTGTCTGGTCAACCACCGCGGACTCAGTGG | 2 |
| | (attP) Site (attP-GA) | TGTACGGTACAAACC | |
| | attB-GT Site | GGCTTGTCGACGACGGCGGTCTCCGTCGTCAGG | 3 |
| | | ATCAT | |
| | attP-GT Site | GGTTTGTCTGGTCAACCACCGCGGTCTCAGTGG | 4 |
| | | TGTACGGTACAAACC | |
| φC31 | attB Site | CGGTGCGGGTGCCAGGGCGTGCCCTTGGCTCCC | 5 |
| | | CGGGCGCGTACTCCAC | |
| | AttP Site | GTGCCCCAACTGGGGTAACCTTTGAGTTCTCTC | 6 |
| | | AGTTGGGGG | |

In certain embodiments, the transfer polynucleotide comprises a recombinase recognition site that is recognized by a Bxb1 recombinase. In certain embodiments, transfer polynucleotides of the disclosure comprise a recombinase recognition site that is recognized by a Bxb1 recombinase, such as an attB, attP, attP-GT, attP-GA, attB-GT, or attB-GA site.

In some embodiments, the transfer polynucleotide comprises an attB site. In some embodiments, the transfer polynucleotide comprises an attB site comprising a nucleotide sequence at least 95%, 96%, 97%, 98%, 99%, or 100% identical to the nucleotide sequence set forth in SEQ ID NO: 1. In some embodiments, the transfer polynucleotide comprises an attB site comprising the nucleotide sequence set forth in SEQ ID NO: 1. In some embodiments, the transfer polynucleotide comprises an attB site consisting of a nucleotide sequence at least 95%, 96%, 97%, 98%, 99%, or 100% identical to the nucleotide sequence set forth in SEQ ID NO: 1. In some embodiments, the transfer polynucleotide comprises an attB site consisting of the nucleotide sequence set forth in SEQ ID NO: 1.

In some embodiments, the transfer polynucleotide comprises an attP site. In some embodiments, the transfer polynucleotide comprises an attP site comprising a nucleotide sequence at least 95%, 96%, 97%, 98%, 99%, or 100% identical to the nucleotide sequence set forth in SEQ ID NO: 2. In some embodiments, the transfer polynucleotide comprises an attP site comprising the nucleotide sequence set forth in SEQ ID NO: 2. In some embodiments, the transfer polynucleotide comprises an attP site consisting of a nucleotide sequence at least 95%, 96%, 97%, 98%, 99%, or 100% identical to the nucleotide sequence set forth in SEQ ID NO: 2. In some embodiments, the transfer polynucleotide comprises an attP site consisting of the nucleotide sequence set forth in SEQ ID NO: 2.

In some embodiments, the transfer polynucleotide comprises an attB site. In some embodiments, the transfer polynucleotide comprises an attB site comprising a nucleotide sequence at least 95%, 96%, 97%, 98%, 99%, or 100% identical to the nucleotide sequence set forth in SEQ ID NO: 3. In some embodiments, the transfer polynucleotide comprises an attB site comprising the nucleotide sequence set forth in SEQ ID NO: 3. In some embodiments, the transfer polynucleotide comprises an attB site consisting of a nucleotide sequence at least 95%, 96%, 97%, 98%, 99%, or 100% identical to the nucleotide sequence set forth in SEQ ID NO: 3. In some embodiments, the transfer polynucleotide comprises an attB site consisting of the nucleotide sequence set forth in SEQ ID NO: 3.

In some embodiments, the transfer polynucleotide comprises an attP site. In some embodiments, the transfer polynucleotide comprises an attP site comprising a nucleotide sequence at least 95%, 96%, 97%, 98%, 99%, or 100% identical to the nucleotide sequence set forth in SEQ ID NO: 4. In some embodiments, the transfer polynucleotide comprises an attP site comprising the nucleotide sequence set forth in SEQ ID NO: 4. In some embodiments, the transfer polynucleotide comprises an attP site consisting of a nucleotide sequence at least 95%, 96%, 97%, 98%, 99%, or 100% identical to the nucleotide sequence set forth in SEQ ID NO: 4. In some embodiments, the transfer polynucleotide comprises an attP site consisting of the nucleotide sequence set forth in SEQ ID NO: 4.

In some embodiments, the recombinase recognition site in a transfer polynucleotide (e.g., transfer plasmid) is a cognate partner site of the recombinase recognition site in a landing pad polynucleotide (e.g., a landing pad plasmid or integrated into a cell) of the disclosure (e.g., a transfer polynucleotide of the disclosure that is to be integrated into the landing pad) (e.g., that are part of the same system (e.g., described herein)). For example, the recombinase recognition site in a transfer polynucleotide (e.g., transfer plasmid) is an attB site when the recombinase recognition site in a landing pad polynucleotide (e.g., a landing pad plasmid or integrated into a cell) of the disclosure is an attP site (e.g., that are part of the same system (e.g., described herein)).

In some embodiments, the transfer polynucleotide comprises an attB site and a corresponding landing pad polynucleotide (e.g., part of a system described herein) comprises an attP site. In some embodiments, the transfer polynucleotide comprises an attB site comprising a nucleotide sequence at least 95%, 96%, 97%, 98%, 99%, or 100% identical to the nucleotide sequence set forth in SEQ ID NO: 1; and a corresponding landing pad polynucleotide (e.g., part of a system described herein) comprises an attP site comprising a nucleotide sequence at least 95%, 96%, 97%, 98%, 99%, or 100% identical to the nucleotide sequence set forth in SEQ ID NO: 2. In some embodiments, the transfer polynucleotide comprises an attB site comprising the nucleotide sequence set forth in SEQ ID NO: 1; and a corresponding landing pad polynucleotide (e.g., part of a system described herein) comprises an attP site comprising the nucleotide sequence set forth in SEQ ID NO: 2. In some embodiments, the transfer polynucleotide comprises an attB site consisting of a nucleotide sequence at least 95%, 96%, 97%, 98%, 99%, or 100% identical to the nucleotide sequence set forth in SEQ ID NO: 1; and a corresponding landing pad polynucleotide (e.g., part of a system described herein) comprises an attP site consisting of a nucleotide sequence at least 95%, 96%, 97%, 98%, 99%, or 100% identical to the nucleotide sequence set forth in SEQ ID NO: 2. In some embodiments, the transfer polynucleotide comprises an attB site consisting of the nucleotide sequence set forth in SEQ ID NO: 1; and a corresponding landing pad polynucleotide (e.g., part of a system described herein) comprises an attP site consisting of the nucleotide sequence set forth in SEQ ID NO: 2.

In some embodiments, the transfer polynucleotide comprises an attP site and a corresponding landing pad polynucleotide (e.g., part of a system described herein) comprises an attB site. In some embodiments, the transfer polynucleotide comprises an attP site comprising a nucleotide sequence at least 95%, 96%, 97%, 98%, 99%, or 100% identical to the nucleotide sequence set forth in SEQ ID NO: 2; and a corresponding landing pad polynucleotide (e.g., part of a system described herein) comprises an attB site comprising a nucleotide sequence at least 95%, 96%, 97%, 98%, 99%, or 100% identical to the nucleotide sequence set forth in SEQ ID NO: 1. In some embodiments, the transfer polynucleotide comprises an attP site comprising the nucleotide sequence set forth in SEQ ID NO: 2; and a corresponding landing pad polynucleotide (e.g., part of a system described herein) comprises an attB site comprising the nucleotide sequence set forth in SEQ ID NO: 1. In some embodiments, the transfer polynucleotide comprises an attP site consisting of a nucleotide sequence at least 95%, 96%, 97%, 98%, 99%, or 100% identical to the nucleotide sequence set forth in SEQ ID NO: 2; and a corresponding landing pad polynucleotide (e.g., part of a system described herein) comprises an attB site consisting of a nucleotide sequence at least 95%, 96%, 97%, 98%, 99%, or 100% identical to the nucleotide sequence set forth in SEQ ID NO: 1. In some embodiments, the transfer polynucleotide comprises an attP site consisting of the nucleotide sequence set forth in SEQ ID NO: 2; and a corresponding landing pad polynucleotide (e.g., part of a system described herein) comprises an attB site consisting of the nucleotide sequence set forth in SEQ ID NO: 1.

In some embodiments, the transfer polynucleotide comprises an attB site and a corresponding landing pad polynucleotide (e.g., part of a system described herein) comprises an attP site. In some embodiments, the transfer polynucleotide comprises an attB site comprising a nucleotide sequence at least 95%, 96%, 97%, 98%, 99%, or 100% identical to the nucleotide sequence set forth in SEQ ID NO: 3; and a corresponding landing pad polynucleotide (e.g., part of a system described herein) comprises an attP site comprising a nucleotide sequence at least 95%, 96%, 97%, 98%, 99%, or 100% identical to the nucleotide sequence set forth in SEQ ID NO: 4. In some embodiments, the transfer polynucleotide comprises an attB site comprising the nucleotide sequence set forth in SEQ ID NO: 3; and a corresponding landing pad polynucleotide (e.g., part of a system described herein) comprises an attP site comprising the nucleotide sequence set forth in SEQ ID NO: 4. In some embodiments, the transfer polynucleotide comprises an attB site consisting of a nucleotide sequence at least 95%, 96%, 97%, 98%, 99%, or 100% identical to the nucleotide sequence set forth in SEQ ID NO: 3; and a corresponding landing pad polynucleotide (e.g., part of a system described herein) comprises an attP site consisting of a nucleotide sequence at least 95%, 96%, 97%, 98%, 99%, or 100% identical to the nucleotide sequence set forth in SEQ ID NO: 4. In some embodiments, the transfer polynucleotide comprises an attB site consisting of the nucleotide sequence set forth in SEQ ID NO: 3; and a corresponding landing pad polynucleotide (e.g., part of a system described herein) comprises an attP site consisting of the nucleotide sequence set forth in SEQ ID NO: 4.

In some embodiments, the transfer polynucleotide comprises an attP site and a corresponding landing pad polynucleotide (e.g., part of a system described herein) comprises an attB site. In some embodiments, the transfer polynucleotide comprises an attP site comprising a nucleotide sequence at least 95%, 96%, 97%, 98%, 99%, or 100% identical to the nucleotide sequence set forth in SEQ ID NO: 4; and a corresponding landing pad polynucleotide (e.g., part of a system described herein) comprises an attB site comprising a nucleotide sequence at least 95%, 96%, 97%, 98%, 99%, or 100% identical to the nucleotide sequence set forth in SEQ ID NO: 3. In some embodiments, the transfer polynucleotide comprises an attP site comprising the nucleotide sequence set forth in SEQ ID NO: 4; and a corresponding landing pad polynucleotide (e.g., part of a system described herein) comprises an attB site comprising the nucleotide sequence set forth in SEQ ID NO: 3. In some embodiments, the transfer polynucleotide comprises an attP site consisting of a nucleotide sequence at least 95%, 96%, 97%, 98%, 99%, or 100% identical to the nucleotide sequence set forth in SEQ ID NO: 4; and a corresponding landing pad polynucleotide (e.g., part of a system described herein) comprises an attB site consisting of a nucleotide sequence at least 95%, 96%, 97%, 98%, 99%, or 100% identical to the nucleotide sequence set forth in SEQ ID NO: 3. In some embodiments, the transfer polynucleotide comprises an attP site consisting of the nucleotide sequence set forth in SEQ ID NO: 4; and a corresponding landing pad polynucleotide (e.g., part of a system described herein) comprises an attB site consisting of the nucleotide sequence set forth in SEQ ID NO: 3.

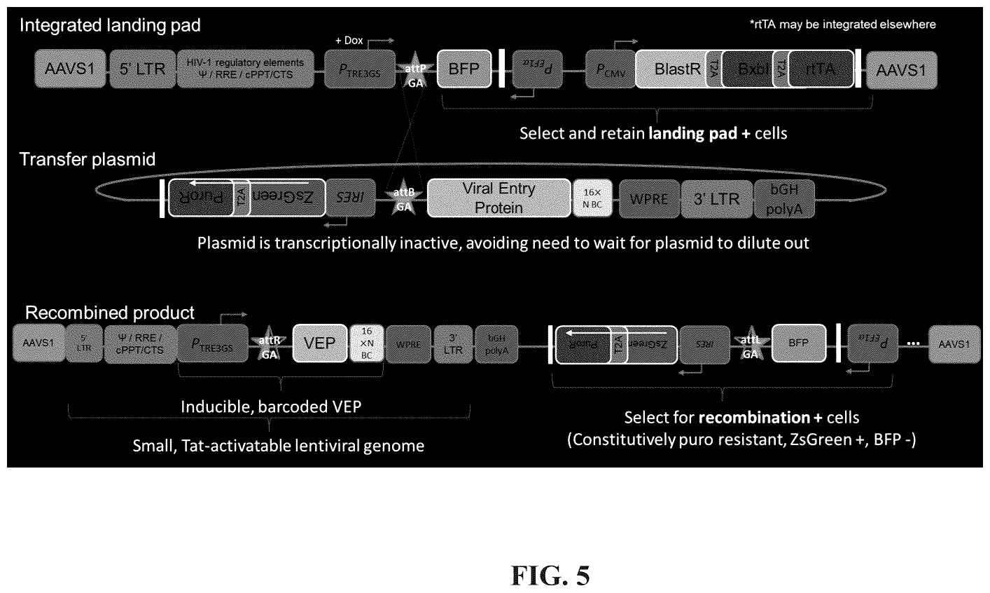

It will be clear to a person of ordinary skill in the art that mechanistically, attB and attP integrative recombination forms attL and attR sites (in the recombined product). This is shown, e.g., in FIGS. 5-8 in the exemplary recombined products (bottom schematic of each of FIGS. 5-8).

### 5.2.5 Selectable Marker Genes

In preferred embodiments, the transfer polynucleotide (e.g., transfer plasmid) comprises one or more (e.g., 1, 2, or 3, or more) selectable marker genes. The one or more selectable marker genes can be utilized for positive selection of transfer polynucleotides that have integrated into the DNA of a cell (e.g., transfer polynucleotides that have integrated into a landing pad described herein in a cell's genome).

Various selectable marker genes are known in the art and a person of ordinary skill in the art can select one or more suitable selectable marker gene for use in a transfer polynucleotide (e.g., transfer plasmid) described herein. Exemplary selectable marker genes, include, but are not limited to drug resistance genes (e.g., antibiotic resistance genes (e.g., puromycin resistance genes, ampicillin resistance genes, gentamycin resistance genes, streptomycin resistance genes, kanamycin resistance genes, hygromycin resistance genes, cefoxitin resistance genes, amoxicillin resistance genes, tetracycline resistance genes, sulfadiazine resistance genes, chloramphenicol resistance genes, fosfomycin resistance genes, trimethoprim resistance genes, erythromycin resistance genes, rifampicin resistance genes, azithromycin resistance genes, Blasticidin resistance genes)); detectable proteins (e.g., fluorescent proteins (e.g., Green Fluorescent Protein (GFP), Blue Fluorescent Protein (BFP), yellow fluorescent protein (YFP), cyan fluorescent protein (CFP), red fluorescent protein (RFP), Zs Green)); suicide genes (e.g., Herpes simplex virus thymidine kinase (HSV-TK) gene, human inducible caspase 9 (iCasp9) gene, mutant human thymidylate kinase (mTMPK) gene, human CD20 gene).

In some embodiments, the transfer polynucleotide (e.g., transfer plasmid) comprises at least one an antibiotic resistance gene (e.g., a puromycin resistance gene). In some embodiments, the at least one antibiotic resistance gene is a puromycin resistance gene. In some embodiments, the transfer polynucleotide comprises a gene encoding a detectable protein. In some embodiments, the detectable protein is a fluorescent protein. In some embodiments, the fluorescent protein is GFP, BFP, YFP, CFP, RFP, or Zs Green.

In some embodiments, the transfer polynucleotide comprises more than 1 (e.g., 2, 3, 4, 5, or more) selectable marker gene. In some embodiments, the transfer polynucleotide comprises a plurality of selectable marker genes. In some embodiments, at least 2 of the selectable marker genes in the plurality are different types (e.g., on is an antibiotic resistance gene and one encodes a detectable protein). In some embodiments, the transfer polynucleotide comprises at least one antibiotic resistance gene and at least one gene encoding a detectable protein. In some embodiments, the transfer polynucleotide comprises at least one suicide gene (e.g., Herpes simplex virus thymidine kinase (HSV-TK) gene, human inducible caspase 9 (iCasp9) gene, mutant human thymidylate kinase (mTMPK) gene, human CD20 gene).

In some embodiments, any selectable marker genes within a transfer polynucleotide (e.g., transfer plasmid) are different from any selectable marker genes within a landing pad polynucleotide described herein (e.g., that are part of a system described herein). As such, the integration of a landing pad into the genomic DNA of a cell could be selected for separately from the integration of a transfer polynucleotide described herein into an integrated landing pad.

### 5.2.6 Gene Regulatory Elements

In preferred embodiments, the transfer polynucleotide (e.g., transfer plasmid) comprises one or more (e.g., 1, 2, 3, 4, 5, or more) gene regulatory elements.

Exemplary gene regulatory elements include, but are not limited to, e.g., promoters, enhancers, internal ribosome entry sites (IRESs), 2A sequences, viral posttranscriptional regulatory elements (e.g., WPRE), transcription termination sequences (e.g., SV40, hGH, BGH, rbGlob terminators), and polyadenylation signal sequences (e.g., polyA sequence).

In some embodiments, the transfer polynucleotide comprises one or more of a promoter; an enhancer; an IRES; a viral posttranscriptional regulatory element (e.g., WPRE); a transcription termination sequence (e.g., SV40, hGH, BGH, rbGlob terminators); a polyadenylation signal sequence (e.g., polyA sequence); and/or a polynucleotide sequence encoding a cleavable peptide, such as self-cleaving peptides (e.g., 2A peptides, e.g., T2A, P2A, E2A, or F2A peptides); or any combination of the foregoing.

In some embodiments, the transfer polynucleotide comprises a promoter. In some embodiments, the transfer polynucleotide comprises an enhancer. In some embodiments, the transfer polynucleotide comprises an IRES. In some embodiments, the transfer polynucleotide comprises a polyA. In some embodiments, the transfer polynucleotide comprises a viral posttranscriptional regulatory element. In some embodiments, the viral posttranscriptional regulatory element is a Woodchuck Hepatitis Virus Posttranscriptional Regulatory Element (WPRE). In some embodiments, the transfer polynucleotide comprises a transcription termination sequence (e.g., (SV40, hGH, BGH, rbGlob terminators). In some embodiments, the transfer polynucleotide comprises a polyadenylation signal sequence (e.g., polyA sequence). In some embodiments, the transfer polynucleotide comprises a polynucleotide sequence encoding a cleavable peptide, such as self-cleaving peptides (e.g., 2A peptides, e.g., T2A, P2A, E2A, or F2A peptides). 2A peptides are typically positioned between protein coding polynucleotide sequences in order to induce ribosomal skipping during translation.

In certain embodiments, the transfer polynucleotide (e.g., transfer plasmid) comprises an IRES operably connected to one or more selectable marker genes (e.g., described herein). In certain embodiments, the transfer polynucleotide (e.g., transfer plasmid) comprises a plurality of selectable maker genes (e.g., described herein), wherein each selectable marker gene of the plurality is separated by a 2A element (e.g., a T2A, P2A, E2A, or F2A element).

In some embodiments, the transfer polynucleotide (e.g., transfer plasmid) does not contain a promoter. In some embodiments, the transfer polynucleotide (e.g., transfer plasmid) does not contain an enhancer. In some embodiments, the transfer polynucleotide (e.g., transfer plasmid) does not contain a promoter or an enhancer.

In some embodiments, the polynucleotide encoding the protein of interest of the transfer polynucleotide (e.g., transfer plasmid) is not operably connected to a promoter, enhancer, or IRES. In some embodiments, the polynucleotide encoding the protein of interest of the transfer polynucleotide (e.g., transfer plasmid) is not operably connected to a promoter. In some embodiments, the polynucleotide encoding the protein of interest of the transfer polynucleotide is not operably connected to an (e.g., transfer plasmid) enhancer. In some embodiments, the polynucleotide encoding the protein of interest of the transfer polynucleotide (e.g., transfer plasmid) is not operably connected to an IRES.

### 5.2.7 Partial Viral Genome

In some embodiments, transfer polynucleotides described herein do not contain a partial viral genome. In some embodiments, transfer polynucleotides described herein comprise a partial viral genome. The partial viral genome can be naturally occurring or a variant of a naturally occurring partial viral genome.

The partial viral genome can be derived from be any virus whose genome can be activated (e.g., upon reconstitution in vivo). Exemplary viruses include, e.g., retroviruses (e.g., lentiviruses (e.g., HIV)), adenoviruses, parvoviruses (e.g., adeno-associated virus), and viruses of family orthoherpesviridae viruses (e.g., herpes viruses, e.g., herpes simplex virus).

In some embodiments, the partial viral genome is a partial retrovirus genome. In some embodiments, the partial virus retrovirus genome is a partial lentivirus genome (e.g., a partial HIV genome). In some embodiments, the partial virus retrovirus genome is a partial HIV genome. In some embodiments, the partial virus genome is a partial adenovirus genome. In some embodiments, the partial viral genome is a partial parvovirus virus genome. In some embodiments, the partial virus genome is a partial adeno-associated virus genome. In some embodiments, the partial viral genome is a partial genome from a virus from the orthoherpesviridae family. In some embodiments, the partial viral genome is a partial herpes virus genome. In some embodiments, the partial viral genome is a partial herpes simplex virus genome.

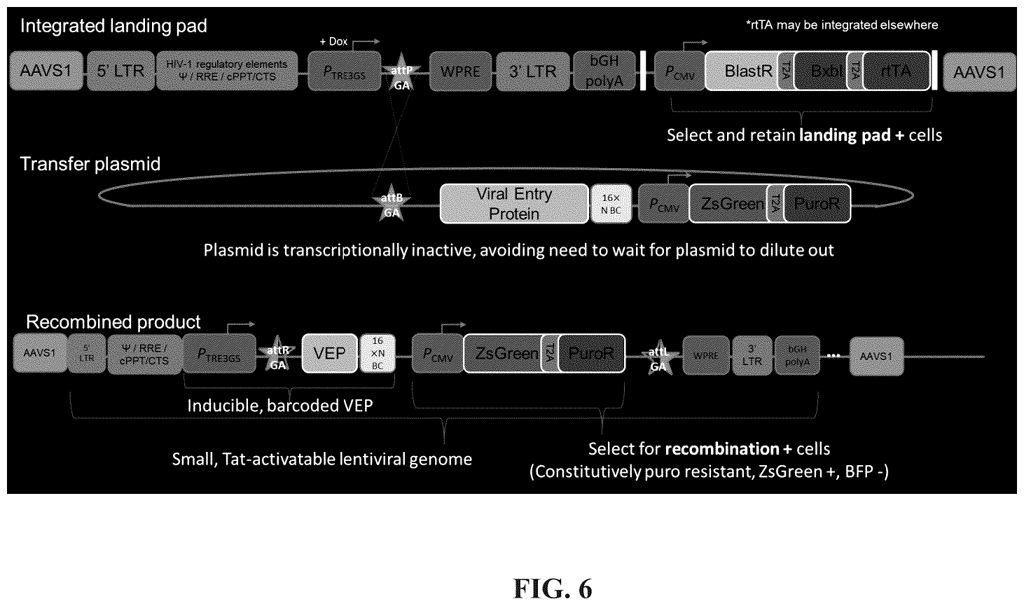

In some embodiments, the partial viral genome comprises or consists of one or more viral long terminal repeat (LTR) (or a variant, fragment, and/or component thereof). In some embodiments, the partial virus genome in the transfer polynucleotide (e.g., transfer plasmid) of the disclosure has one LTR (see, e.g., FIG. 5). In some embodiments, the partial virus genome in a landing pad of the disclosure has no LTRs, for example, when a corresponding landing pad (e.g., landing pad plasmid) (e.g., of the same system) has two LTRs (e.g., a 5′ and a 3′ LTR)) (see, e.g., FIG. 6).

In some embodiments, the partial viral genome comprises a 5′ LTR. In some embodiments, the partial viral genome comprises a 3′ LTR. In some embodiments, the partial viral genome comprises a 5′ LTR and a 3′ LTR. In some embodiments, the partial viral genome comprises a 5′ LTR and lacks a 3′ LTR. In some embodiments, the partial viral genome comprises a 3′ LTR and lacks a 5′ LTR. In some embodiments, the partial viral genome consists of a 5′ LTR. In some embodiments, the partial viral genome consists of a 3′ LTR. In some embodiments, the partial viral genome consists of a 5′ LTR and lacks a 3′ LTR. In some embodiments, the partial viral genome consists of a 3′ LTR and lacks a 5′ LTR.

In embodiments, wherein the partial viral genome contains a 3′ LTR, the 3′ LTR can be a reference 3′ LTR (e.g., wild type) or a variant thereof. In some embodiments, the 3′ LTR comprises a full length U3 region (i.e., the 3′ LTR does not have a deletion of any part of the U3 region). In some embodiments, the 3′ LTR comprises a portion of U3 region (i.e., the 3′ LTR has a deletion of part of the U3 region). In some embodiments, the 3′ LTR comprises a functional deletion of at least a portion of the U3 region (i.e., the 3′ LTR has a deletion of at least a portion of the U3 region to render it non-functional). In some embodiments, the 3′ LTR does not contain a U3 region (i.e., the 3′ LTR has a deletion of the entire U3 region). In some embodiments, the comprises a functional variation (e.g., one or more nucleotide substitution) of at least a portion of the U3 region (i.e., the 3′ LTR has a deletion of at least a portion of the U3 region to render it non-functional).

The partial viral genome can include additional genes encoding one or more viral proteins. For example, the partial viral genome can also include one or more viral structural gene, regulatory gene, and/or accessory gene. For example, in embodiments wherein the partial viral genome is an HIV partial viral genome, the partial viral genome of the transfer polynucleotide may comprise any one or more HIV virus protein (e.g., gag, pol, env genes), HIV virus regulatory genes (e.g., tat, rev genes), and/or HIV virus accessory genes (e.g., HIV-1 vif, vpr, vpu, nef genes).

As described elsewhere herein, in embodiments, wherein the transfer polynucleotide comprises a partial viral genome; the corresponding landing pad (e.g., in a system described herein or integrated into a cell described herein) (e.g., that are part of the same system) can comprise a corresponding part of the same partial viral genome. As such, integration of the transfer polynucleotide (e.g., transfer plasmid) at the recombinase recognition site in the recombinase landing pad results in a reconstituted, or reconstructed, viral genome (e.g., comprising two LTRs, viral protein genes, viral regulatory genes and/or viral accessory genes). The partial viral genome in the transfer polynucleotide (e.g., transfer plasmid) (e.g., that are part of the same system) that is to be integrated into a corresponding landing pad is preferably from the same type of virus as the partial viral genome in the corresponding landing pad.

For example, in embodiments, wherein the transfer polynucleotide described herein comprises a partial viral genome comprising a 3′ LTR or a variant, fragment, or component thereof); the corresponding landing pad (e.g., in a system described herein or integrated into a cell described herein) may comprise a partial viral genome from the corresponding 5′LTR (or a variant, fragment, or component thereof) of the same viral genome. For example, in some embodiments, the transfer polynucleotide described herein comprises a partial HIV viral genome comprising an HIV 3′ LTR (or a variant thereof); and the corresponding landing pad (e.g., in a system described herein or integrated into a cell described herein) comprises a partial viral genome comprising the corresponding HIV 5′ LTR or a variant, fragment, or component thereof).

### 5.2.8 Integrated Transfer Polynucleotides

As described throughout, the transfer polynucleotides described herein (see, e.g., § 5.2) can be isolated (e.g., not integrated into a landing pad) (e.g., a transfer plasmid) or integrated in a landing pad (e.g., a landing pad integrated into a cell's genomic DNA).

In some embodiments, the transfer polynucleotide is isolated.

In some embodiments, the transfer polynucleotide is integrated into a landing pad (e.g., a landing pad integrated into a cell's genomic DNA). A person of ordinary skill in the art would understand that introduction of e.g., a transfer plasmid (e.g., described herein) and subsequent integration may result in only a portion of an isolated transfer plasmid being integrated into the landing pad.

### 5.3 Libraries of Transfer Polynucleotides (e.g., Transfer Plasmids)

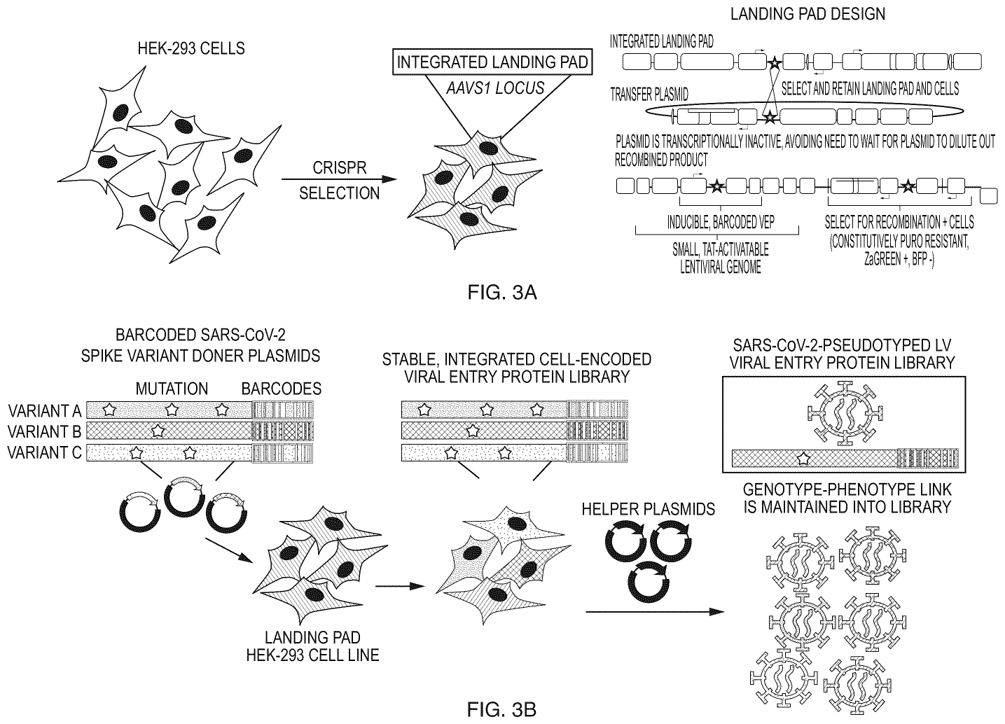

Also provided herein are, inter alia, pluralities (e.g., collections, libraries) of transfer polynucleotides (e.g., transfer plasmids) described herein. For example, FIG. 3B (left) shows a plurality (e.g., a collection, a library) of transfer polynucleotides (e.g., transfer plasmids) each encoding a different protein (e.g., viral entry protein).

The transfer polynucleotides (e.g., transfer plasmids) in the plurality can be identical or differ in sequence. In some embodiments, other than the polynucleotide sequence encoding the protein of interest, the sequence of the transfer polynucleotides in the plurality (e.g., a collection, a library) is substantially identical. In some embodiments, the sequence of the transfer polynucleotides in the plurality (e.g., a collection, a library) is substantially identical outside of the polynucleotide sequence encoding the protein of interest. In some embodiments, other than the polynucleotide sequence encoding the protein of interest, the sequence of the transfer polynucleotides in the plurality (e.g., a collection, a library) is identical. In some embodiments, the sequence of the transfer polynucleotides in the plurality (e.g., a collection, a library) is identical outside of the polynucleotide sequence encoding the protein of interest. In some embodiments, other than the polynucleotide sequence encoding the protein of interest, the sequence of the transfer polynucleotides in the plurality (e.g., a collection, a library) is at least 95%, 96%, 97%, 98%, 99% or 100% identical. In some embodiments, the sequence of the transfer polynucleotides in the plurality (e.g., a collection, a library) is at least 95%, 96%, 97%, 98%, 99% or 100% identical outside of the polynucleotide sequence encoding the protein of interest.

In some embodiments, the transfer polynucleotides (e.g., transfer plasmids) in the plurality collectively encode a plurality (e.g., a collection, a library) of viral entry proteins. In some embodiments, each transfer polynucleotide (e.g., transfer plasmid) in the plurality encodes a different viral entry protein, relative to the other transfer polynucleotides (e.g., transfer plasmid) in the plurality.

In some embodiments, the transfer polynucleotides (e.g., transfer plasmids) in the plurality collectively encode a plurality (e.g., a collection, a library) of different viral entry proteins. In some embodiments, each transfer polynucleotide (e.g., transfer plasmid) in the plurality encodes a different viral entry protein, relative to the other transfer polynucleotides (e.g., transfer plasmid) in the plurality.

In some embodiments, the plurality of transfer polynucleotides (e.g., transfer plasmids) comprises a plurality of transfer polynucleotides (e.g., transfer plasmids) collectively encoding a plurality (e.g., a collection, a library) of different variants of a reference protein (e.g., a reference viral entry protein); and optionally a transfer polynucleotide (e.g., transfer plasmid) encoding the reference protein (e.g., the reference viral entry protein). In some embodiments, the plurality of transfer polynucleotides (e.g., transfer plasmids) comprises a plurality of transfer polynucleotides (e.g., transfer plasmids) collectively encoding a plurality (e.g., a collection, a library) of different variants of a reference protein (e.g., a reference viral entry protein); and a transfer polynucleotide (e.g., transfer plasmid) encoding the reference protein (e.g., the reference viral entry protein).

In some embodiments, the plurality of transfer polynucleotides (e.g., transfer plasmids) comprises (a) a plurality of transfer polynucleotides (e.g., transfer plasmids) collectively encoding a plurality (e.g., a collection, a library) of different variants of a reference viral entry protein; and (b) optionally a transfer polynucleotide (e.g., transfer plasmid) encoding the reference viral entry protein. In some embodiments, the plurality of transfer polynucleotides (e.g., transfer plasmids) comprises (a) a plurality of transfer polynucleotides (e.g., transfer plasmids) collectively encoding a plurality (e.g., a collection, a library) of different variants of a reference viral entry protein; and (b) a transfer polynucleotide (e.g., transfer plasmid) encoding the reference viral entry protein.

The reference protein can be any peptide or protein (e.g., an enzyme, a structural protein, a targeting protein, a signaling protein, an antibody or antigen-binding fragment of an antibody). For example, any protein of interest described herein (see, e.g., § 5.2.3). In some embodiments, the reference protein is a protein of interest described in § 5.2.3, 5.2.3.1.

In some embodiments, the reference protein is a non-viral protein (e.g., a cell targeting protein or peptide, e.g., an antibody (e.g., a scFv, a Fab)).

In preferred embodiments, the reference protein is a viral protein. In certain preferred embodiments, the viral protein is a viral entry protein (e.g., described herein, see, e.g., § 5.2.3.1) (e.g., a spike protein of a SARS virus (e.g., a SARS-COV-2 virus); an HA protein of an influenza virus). In certain embodiments, the viral entry protein is a viral entry protein described in § 5.2.3.1. In certain embodiments, the viral entry protein is a SARS-COV-2 spike protein. In certain embodiments, the viral entry protein is an influenza HA protein.

In some embodiments, the plurality (e.g., collection, library) of transfer polynucleotides are transfer vectors (e.g., viral vectors, non-viral vectors, gene delivery vectors, plasmids). In some embodiments, the plurality (e.g., collection, library) of transfer polynucleotides are transfer plasmids. In some embodiments, the plurality (e.g., collection, library) of transfer polynucleotides are transfer non-viral vectors. In some embodiments, the plurality (e.g., collection, library) of transfer polynucleotides are transfer viral vectors. In some embodiments, the plurality (e.g., collection, library) of transfer polynucleotides are transfer gene delivery vectors.

A plurality of transfer polynucleotides can be generated using a variety of methods that are well known in the art, including library generation methods that are well known in the art. Examples of methods of generating libraries include those described in WO2014/201416 A1 and WO2020/006494, the entire contents of which are incorporated herein by reference for all purposes. In some embodiments, a plurality of amino acid sequences is generated in silico prior to making transfer polynucleotides (e.g., transfer plasmids of the disclosure) that collectively encode the proteins (e.g., different proteins) (e.g., recombinantly, synthetically).

In some embodiments, the plurality (e.g., library, collection) comprises at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or more different transfer polynucleotides (e.g., transfer plasmids) (i.e., encoding a different protein of interest). In some embodiments, the plurality (e.g., library, collection) comprises more than 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or more different transfer polynucleotides (e.g., transfer plasmids) (i.e., encoding a different protein of interest).

### 5.4 Landing Pad Polynucleotides (e.g., Landing Pad Plasmids)

Provided herein are, inter alia, landing pad polynucleotides (e.g., landing pad plasmids) (also referred to herein as recombinase landing pad polynucleotides) (e.g., recombinase landing pad plasmids) that function to enable site-specific integration of a transfer polynucleotide (e.g., described herein) (or a portion thereof) into the genome of a cell.

The landing pad polynucleotides described herein can be isolated (e.g., not integrated in genomic DNA) (e.g., a landing pad plasmid) or integrated in a cell's genomic DNA (see, e.g., § 5.4.8) (e.g., a landing pad).

The landing pad polynucleotide can take any suitable form of a polynucleotide (e.g., described herein, see, e.g., § 5.8) or a polynucleotide incorporated in a vector (e.g., a plasmid) (e.g., a vector described herein, see, e.g., § 5.9).

In some embodiments, the landing pad polynucleotide is double stranded. In some embodiments, the landing pad polynucleotide is single stranded. In some embodiments, the landing pad polynucleotide is linear. In some embodiments, the landing pad polynucleotide is circular. In some preferred embodiments, the landing pad polynucleotide is circular. The landing pad polynucleotide can comprise DNA nucleotides and/or RNA nucleotides. In some embodiments, the landing pad polynucleotide comprises one or more non-natural nucleotides. In some preferred embodiments, the landing pad polynucleotide is a DNA polynucleotide. In some preferred embodiments, the landing pad polynucleotide is a circular double-stranded DNA molecule. In some embodiments, the landing pad polynucleotide is a circular single-stranded DNA molecule.

In some embodiments, the landing pad polynucleotide is incorporated into a vector (i.e., a landing pad vector) (e.g., a gene delivery vector) (e.g., a vector described herein, see, e.g., § 5.9). In some embodiments, the vector is a plasmid (i.e., a landing pad plasmid). In some embodiments, the vector is a viral vector (i.e., a landing pad viral vector). Suitable vectors (e.g., gene delivery vectors, plasmids, viral vectors, and non-viral vectors) for use in preparing landing pad polynucleotides of the disclosure are known in the art and are commercially available.

### 5.4.1 Landing Pad Polynucleotide (e.g., Landing Pad Plasmid) Structure

In some embodiments, the landing pad polynucleotide (e.g., landing pad plasmid) comprises any one or more of (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); and/or (v) a partial viral genome (see, e.g., § 5.4.4).

In some embodiments, the landing pad polynucleotide (e.g., landing pad plasmid) comprises (i) a recombinase recognition site (see, e.g., § 5.4.3); and any one or more of (ii) one or more selectable marker genes (see, e.g., § 5.4.5); (iii) one or more gene regulatory elements (see, e.g., § 5.4.6)); and/or (iv) a partial viral genome (see, e.g., § 5.4.4).

In some embodiments, the landing pad polynucleotide (e.g., landing pad plasmid) comprises (i) a recombinase recognition site (see, e.g., § 5.4.3); (ii) one or more selectable marker genes (see, e.g., § 5.4.5); (iii) one or more gene regulatory elements (see, e.g., § 5.4.6)); and (iv) a partial viral genome (see, e.g., § 5.4.4).

In some embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); and (v) a partial viral genome (see, e.g., § 5.4.4).

In some embodiments, the landing pad polynucleotide (e.g., landing pad plasmid) comprises (i) a recombinase recognition site (see, e.g., § 5.4.3); and any one or more of (ii) one or more gene regulatory elements (see, e.g., § 5.4.6)); and/or (iii) a partial viral genome (see, e.g., § 5.4.4).

In some embodiments, the landing pad polynucleotide (e.g., landing pad plasmid) comprises (i) a recombinase recognition site (see, e.g., § 5.4.3); (ii) one or more gene regulatory elements (see, e.g., § 5.4.6)); and (iii) a partial viral genome (see, e.g., § 5.4.4).

In some embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more gene regulatory elements (see, e.g., § 5.4.6)); and (iv) a partial viral genome (see, e.g., § 5.4.4).

In some preferred embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); and/or (v) a partial viral genome (see, e.g., § 5.4.4).

In some embodiments, the landing pad polynucleotide (e.g., landing pad plasmid) comprises any one or more of (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); (v) a partial viral genome (see, e.g., § 5.4.4); and/or (vi) right homology arm and a left homology arm.

In some embodiments, the landing pad polynucleotide (e.g., landing pad plasmid) comprises (i) a recombinase recognition site (see, e.g., § 5.4.3); and any one or more of (ii) one or more selectable marker genes (see, e.g., § 5.4.5); (iii) one or more gene regulatory elements (see, e.g., § 5.4.6)); (iv) a partial viral genome (see, e.g., § 5.4.4); and/or (v) a right homology arm and a left homology arm.

In some embodiments, the landing pad polynucleotide (e.g., landing pad plasmid) comprises (i) a recombinase recognition site (see, e.g., § 5.4.3); (ii) one or more selectable marker genes (see, e.g., § 5.4.5); (iii) one or more gene regulatory elements (see, e.g., § 5.4.6)); (iv) a partial viral genome (see, e.g., § 5.4.4); and (v) right homology arm and a left homology arm.

In some embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); (v) a partial viral genome (see, e.g., § 5.4.4); and (vi) a right homology arm and a left homology arm.

In some embodiments, the landing pad polynucleotide (e.g., landing pad plasmid) comprises (i) a recombinase recognition site (see, e.g., § 5.4.3); and any one or more of (ii) one or more gene regulatory elements (see, e.g., § 5.4.6)); (iii) a partial viral genome (see, e.g., § 5.4.4); and/or (iv) a right homology arm and a left homology arm.

In some embodiments, the landing pad polynucleotide (e.g., landing pad plasmid) comprises (i) a recombinase recognition site (see, e.g., § 5.4.3); (ii) one or more gene regulatory elements (see, e.g., § 5.4.6)); (iii) a partial viral genome (see, e.g., § 5.4.4); and (iv) a right homology arm and a left homology arm.

In some embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more gene regulatory elements (see, e.g., § 5.4.6)); (iv) a partial viral genome (see, e.g., § 5.4.4); and (v) a right homology arm and a left homology arm.

In some preferred embodiments, the landing pad polynucleotide (e.g., landing pad plasmid) comprises (i) a recombinase recognition site (see, e.g., § 5.4.3); (ii) a polynucleotide encoding a site-specific recombinase; (iii) one or more selectable marker genes (see, e.g., § 5.4.5); (iv) one or more gene regulatory elements (see, e.g., § 5.4.6)); (v) a partial viral genome (see, e.g., § 5.4.4); and/or (vi) a right homology arm and a left homology arm.

### 5.4.2 Recombinases

In some preferred embodiments, the landing pad polynucleotide (e.g., landing pad plasmid) comprises a polynucleotide encoding a site-specific recombinase.

Site-specific recombinases are known in the art and described herein. See, also, e.g., Gaj 2014, Durrant 2023, Merrick 2018, and Low 2022.

Exemplary site specific recombinases, include but are not limited to, tyrosine site-specific recombinases (e.g., Cre, Dre, Flp, KD, B2, B3); tyrosine integrases (e.g., λ, HK022, HP01); serine resolvases/invertases (e.g., γδ, ParA, Tn3, Gin); and serine integrases (e.g., φC31, Bxb1, and R4). In specific embodiments, the recombinase is a serine integrase. In specific embodiments, the recombinase is R4. In specific embodiments, the recombinase is φC31. In specific embodiments, the recombinase is Bxb1.

The amino acid sequence of an exemplary site-specific recombinases is provided in Table 3.

TABLE 3
Amino Acid Sequence of Exemplary Site-Specific Recombinases.

| | | SEQ |
|---|---|---|
| Description | Amino Acid Sequence | ID NO |
| Bxb1 | MRALVVIRLSRVTDATTSPERQLESCQQLCAQRGWDVVGVAEDLDVSGAVDP | 7 |
| Uniprot ID: | FDRKRRPNLARWLAFEEQPFDVIVAYRVDRLTRSIRHLQQLVHWAEDHKKLV | |
| Q9B086 | VSATEAHFDTTTPFAAVVIALMGTVAQMELEAIKERNRSAAHFNIRAGKYRG | |
| | SLPPWGYLPTRVDGEWRLVPDPVQRERILEVYHRVVDNHEPLHLVAHDLNRR | |
| | GVLSPKDYFAQLQGREPQGREWSATALKRSMISEAMLGYATLNGKTVRDDDG | |
| | APLVRAEPILTREQLEALRAELVKTSRAKPAVSTPSLLLRVLFCAVCGEPAY | |
| | KFAGGGRKHPRYRCRSMGFPKHCGNGTVAMAEWDAFCEEQVLDLLGDAERLE | |
| | KVWVAGSDSAVELAEVNAELVDLTSLIGSPAYRAGSPQREALDARIAALAAR | |
| | QEELEGLEARPSGWEWRETGQRFGDWWREQDTAAKNTWLRSMNVRLTFDVRG | |
| | GLTRTIDFGDLQEYEQHLRLGSVVERLHTGMS | |
| φC31 | MDTYAGAYDRQSRERENSSAASPATQRSANEDKAADLQREVERDGGRFRFVG | 8 |
| Uniprot ID: | HFSEAPGTSAFGTAERPEFERILNECRAGRLNMIIVYDVSRFSRLKVMDAIP | |
| Q9T221 | IVSELLALGVTIVSTQEGVFRQGNVMDLIHLIMRLDASHKESSLKSAKILDT | |
| | KNLQRELGGYVGGKAPYGFELVSETKEITRNGRMVNVVINKLAHSTTPLTGP | |
| | FEFEPDVIRWWWREIKTHKHLPFKPGSQAAIHPGSITGLCKRMDADAVPTRG | |
| | ETIGKKTASSAWDPATVMRILRDPRIAGFAAEVIYKKKPDGTPTTKIEGYRI | |
| | QRDPITLRPVELDCGPIIEPAEWYELQAWLDGRGRGKGLSRGQAILSAMDKL | |
| | YCECGAVMTSKRGEESIKDSYRCRRRKVVDPSAPGQHEGTCNVSMAALDKFV | |
| | AERIFNKIRHAEGDEETLALLWEAARRFGKLTEAPEKSGERANLVAERADAL | |
| | NALEELYEDRAAGAYDGPVGRKHFRKQQAALTLRQQGAEERLAELEAAEAPK | |
| | LPLDQWFPEDADADPTGPKSWWGRASVDDKRVFVGLFVDKIVVTKSTTGRGQ | |
| | GTPIEKRASITWAKPPTDDDEDDAQDGTEDVAA | |

In some embodiments, the recombinase is a recombinase set forth in Table 3 (or a variant thereof). In some embodiments, the amino acid sequence of the recombinase comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a recombinase set forth in Table 3. In some embodiments, the amino acid sequence of the recombinase comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7. In some embodiments, the amino acid sequence of the recombinase comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8.

It is clear to a person of ordinary skill in the art that the site-specific recombinase pairs with cognate recombinase recognitions sites. As such, a person of ordinary skill in the art can determine suitable pairs of site-specific recombinases and recombinase recognition sites for use in landing pads (and systems) described herein.

### 5.4.3 Recombinase Recognition Sites

In preferred embodiments, the landing pad polynucleotides (e.g., landing pad plasmids) described herein comprise a recombinase recognition site.

As described above (see, e.g., §§ 5.2.4, 5.4.2), site-specific recombinases and their cognate recognition sites are known in the art. For example, exemplary site-specific recombinases, include, but are not limited to, tyrosine recombinases (e.g., Cre, Flp, and λ integrase) and serine recombinases, or serine integrases, which are also known as resolvases (e.g., Bxb1 recombinase/integrase, φC31 integrase, γδ resolvase, and Gin invertase). Exemplary site-specific tyrosine and serine recombinases and their recognition sites are described, for example, in Gaj 2014. Additional examples of site-specific tyrosine and serine recombinases and their cognate attachment sites are disclosed in Durrant 2023 and Merrick 2018. Additional examples of Bxb1 recognition sites include the attP-GT and attP-GA sites described in Low 2022. Additional Bxb1 recognition sites are described in Zhang 2022.

Exemplary recognition sites for Bxb1 recombinase/integrase and φC31 integrase are set forth above in Table 2.

In certain embodiments, the landing pad polynucleotide comprises a recombinase recognition site that is recognized by a Bxb1 recombinase. In certain embodiments, landing pads of the disclosure comprise a recombinase recognition site that is recognized by a Bxb1 recombinase, such as an attB, attP, attP-GT, attP-GA, attB-GT, or attB-GA site.

In some embodiments, the landing pad polynucleotide comprises an attB site. In some embodiments, the landing pad polynucleotide comprises an attB site comprising a nucleotide sequence at least 95%, 96%, 97%, 98%, 99%, or 100% identical to the nucleotide sequence set forth in SEQ ID NO: 1. In some embodiments, the landing pad polynucleotide comprises an attB site comprising the nucleotide sequence set forth in SEQ ID NO: 1. In some embodiments, the landing pad polynucleotide comprises an attB site consisting of a nucleotide sequence at least 95%, 96%, 97%, 98%, 99%, or 100% identical to the nucleotide sequence set forth in SEQ ID NO: 1. In some embodiments, the landing pad polynucleotide comprises an attB site consisting of the nucleotide sequence set forth in SEQ ID NO: 1.

In some embodiments, the landing pad polynucleotide comprises an attP site. In some embodiments, the landing pad polynucleotide comprises an attP site comprising a nucleotide sequence at least 95%, 96%, 97%, 98%, 99%, or 100% identical to the nucleotide sequence set forth in SEQ ID NO: 2. In some embodiments, the landing pad polynucleotide comprises an attP site comprising the nucleotide sequence set forth in SEQ ID NO: 2. In some embodiments, the landing pad polynucleotide comprises an attP site consisting of a nucleotide sequence at least 95%, 96%, 97%, 98%, 99%, or 100% identical to the nucleotide sequence set forth in SEQ ID NO: 2. In some embodiments, the landing pad polynucleotide comprises an attP site consisting of the nucleotide sequence set forth in SEQ ID NO: 2.

In some embodiments, the landing pad polynucleotide comprises an attB site. In some embodiments, the landing pad polynucleotide comprises an attB site comprising a nucleotide sequence at least 95%, 96%, 97%, 98%, 99%, or 100% identical to the nucleotide sequence set forth in SEQ ID NO: 3. In some embodiments, the landing pad polynucleotide comprises an attB site comprising the nucleotide sequence set forth in SEQ ID NO: 3. In some embodiments, the landing pad polynucleotide comprises an attB site consisting of a nucleotide sequence at least 95%, 96%, 97%, 98%, 99%, or 100% identical to the nucleotide sequence set forth in SEQ ID NO: 3. In some embodiments, the landing pad polynucleotide comprises an attB site consisting of the nucleotide sequence set forth in SEQ ID NO: 3.

In some embodiments, the landing pad polynucleotide comprises an attP site. In some embodiments, the landing pad polynucleotide comprises an attP site comprising a nucleotide sequence at least 95%, 96%, 97%, 98%, 99%, or 100% identical to the nucleotide sequence set forth in SEQ ID NO: 4. In some embodiments, the landing pad polynucleotide comprises an attP site comprising the nucleotide sequence set forth in SEQ ID NO: 4. In some embodiments, the landing pad polynucleotide comprises an attP site consisting of a nucleotide sequence at least 95%, 96%, 97%, 98%, 99%, or 100% identical to the nucleotide sequence set forth in SEQ ID NO: 4. In some embodiments, the landing pad polynucleotide comprises an attP site consisting of the nucleotide sequence set forth in SEQ ID NO: 4.

In some embodiments, the recombinase recognition site in a recombinase landing pad is a cognate site of the recombinase recognition site in a transfer polynucleotide (e.g., transfer plasmid) of the disclosure (e.g., a transfer polynucleotide of the disclosure that is to be integrated into the recombinase landing pad) (e.g., part of the same system). For example, the recombinase recognition site in a recombinase landing pad is an attB site when the recombinase recognition site in a transfer polynucleotide (e.g., transfer plasmid) of the disclosure is an attP site.

In some embodiments, the recombinase recognition site in a landing pad (e.g., a landing pad plasmid or integrated into a cell) is a cognate partner site of the recombinase recognition site in a transfer polynucleotide of the disclosure (e.g., a transfer polynucleotide of the disclosure that is to be integrated into the landing pad) (e.g., that are part of the same system (e.g., described herein)). For example, the recombinase recognition site in a landing pad (e.g., a landing pad plasmid or integrated into a cell) is an attB site when the recombinase recognition site in a transfer polynucleotide (e.g., transfer plasmid) of the disclosure is an attP site (e.g., that are part of the same system (e.g., described herein)).

In some embodiments, the landing pad polynucleotide comprises an attB site and a corresponding transfer polynucleotide (e.g., part of a system described herein) comprises an attP site. In some embodiments, the landing pad polynucleotide comprises an attB site comprising a nucleotide sequence at least 95%, 96%, 97%, 98%, 99%, or 100% identical to the nucleotide sequence set forth in SEQ ID NO: 1; and a corresponding transfer polynucleotide (e.g., part of a system described herein) comprises an attP site comprising a nucleotide sequence at least 95%, 96%, 97%, 98%, 99%, or 100% identical to the nucleotide sequence set forth in SEQ ID NO: 2. In some embodiments, the landing pad polynucleotide comprises an attB site comprising the nucleotide sequence set forth in SEQ ID NO: 1; and a corresponding transfer polynucleotide (e.g., part of a system described herein) comprises an attP site comprising the nucleotide sequence set forth in SEQ ID NO: 2. In some embodiments, the landing pad polynucleotide comprises an attB site consisting of a nucleotide sequence at least 95%, 96%, 97%, 98%, 99%, or 100% identical to the nucleotide sequence set forth in SEQ ID NO: 1; and a corresponding transfer polynucleotide (e.g., part of a system described herein) comprises an attP site consisting of a nucleotide sequence at least 95%, 96%, 97%, 98%, 99%, or 100% identical to the nucleotide sequence set forth in SEQ ID NO: 2. In some embodiments, the landing pad polynucleotide comprises an attB site consisting of the nucleotide sequence set forth in SEQ ID NO: 1; and a corresponding transfer polynucleotide (e.g., part of a system described herein) comprises an attP site consisting of the nucleotide sequence set forth in SEQ ID NO: 2.

In some embodiments, the landing pad polynucleotide comprises an attP site and a corresponding transfer polynucleotide (e.g., part of a system described herein) comprises an attB site. In some embodiments, the landing pad polynucleotide comprises an attP site comprising a nucleotide sequence at least 95%, 96%, 97%, 98%, 99%, or 100% identical to the nucleotide sequence set forth in SEQ ID NO: 2; and a corresponding transfer polynucleotide (e.g., part of a system described herein) comprises an attB site comprising a nucleotide sequence at least 95%, 96%, 97%, 98%, 99%, or 100% identical to the nucleotide sequence set forth in SEQ ID NO: 1. In some embodiments, the landing pad polynucleotide comprises an attP site comprising the nucleotide sequence set forth in SEQ ID NO: 2; and a corresponding transfer polynucleotide (e.g., part of a system described herein) comprises an attB site comprising the nucleotide sequence set forth in SEQ ID NO: 1. In some embodiments, the landing pad polynucleotide comprises an attP site consisting of a nucleotide sequence at least 95%, 96%, 97%, 98%, 99%, or 100% identical to the nucleotide sequence set forth in SEQ ID NO: 2; and a corresponding transfer polynucleotide (e.g., part of a system described herein) comprises an attB site consisting of a nucleotide sequence at least 95%, 96%, 97%, 98%, 99%, or 100% identical to the nucleotide sequence set forth in SEQ ID NO: 1. In some embodiments, the landing pad polynucleotide comprises an attP site consisting of the nucleotide sequence set forth in SEQ ID NO: 2; and a corresponding transfer polynucleotide (e.g., part of a system described herein) comprises an attB site consisting of the nucleotide sequence set forth in SEQ ID NO: 1.

In some embodiments, the landing pad polynucleotide comprises an attB site and a corresponding transfer polynucleotide (e.g., part of a system described herein) comprises an attP site. In some embodiments, the landing pad polynucleotide comprises an attB site comprising a nucleotide sequence at least 95%, 96%, 97%, 98%, 99%, or 100% identical to the nucleotide sequence set forth in SEQ ID NO: 3; and a corresponding transfer polynucleotide (e.g., part of a system described herein) comprises an attP site comprising a nucleotide sequence at least 95%, 96%, 97%, 98%, 99%, or 100% identical to the nucleotide sequence set forth in SEQ ID NO: 4. In some embodiments, the landing pad polynucleotide comprises an attB site comprising the nucleotide sequence set forth in SEQ ID NO: 3; and a corresponding transfer polynucleotide (e.g., part of a system described herein) comprises an attP site comprising the nucleotide sequence set forth in SEQ ID NO: 4. In some embodiments, the landing pad polynucleotide comprises an attB site consisting of a nucleotide sequence at least 95%, 96%, 97%, 98%, 99%, or 100% identical to the nucleotide sequence set forth in SEQ ID NO: 3; and a corresponding transfer polynucleotide (e.g., part of a system described herein) comprises an attP site consisting of a nucleotide sequence at least 95%, 96%, 97%, 98%, 99%, or 100% identical to the nucleotide sequence set forth in SEQ ID NO: 4. In some embodiments, the landing pad polynucleotide comprises an attB site consisting of the nucleotide sequence set forth in SEQ ID NO: 3; and a corresponding transfer polynucleotide (e.g., part of a system described herein) comprises an attP site consisting of the nucleotide sequence set forth in SEQ ID NO: 4.

In some embodiments, the landing pad polynucleotide comprises an attP site and a corresponding transfer polynucleotide (e.g., part of a system described herein) comprises an attB site. In some embodiments, the landing pad polynucleotide comprises an attP site comprising a nucleotide sequence at least 95%, 96%, 97%, 98%, 99%, or 100% identical to the nucleotide sequence set forth in SEQ ID NO: 4; and a corresponding transfer polynucleotide (e.g., part of a system described herein) comprises an attB site comprising a nucleotide sequence at least 95%, 96%, 97%, 98%, 99%, or 100% identical to the nucleotide sequence set forth in SEQ ID NO: 3. In some embodiments, the landing pad polynucleotide comprises an attP site comprising the nucleotide sequence set forth in SEQ ID NO: 4; and a corresponding transfer polynucleotide (e.g., part of a system described herein) comprises an attB site comprising the nucleotide sequence set forth in SEQ ID NO: 3. In some embodiments, the landing pad polynucleotide comprises an attP site consisting of a nucleotide sequence at least 95%, 96%, 97%, 98%, 99%, or 100% identical to the nucleotide sequence set forth in SEQ ID NO: 4; and a corresponding transfer polynucleotide (e.g., part of a system described herein) comprises an attB site consisting of a nucleotide sequence at least 95%, 96%, 97%, 98%, 99%, or 100% identical to the nucleotide sequence set forth in SEQ ID NO: 3. In some embodiments, the landing pad polynucleotide comprises an attP site consisting of the nucleotide sequence set forth in SEQ ID NO: 4; and a corresponding transfer polynucleotide (e.g., part of a system described herein) comprises an attB site consisting of the nucleotide sequence set forth in SEQ ID NO: 3.

It will be clear to a person of ordinary skill in the art that mechanistically, attB and attP integrative recombination forms attL and attR sites (in the recombined product). This is shown, e.g., in FIGS. 5-8 in the exemplary recombined products (bottom schematic of each of FIGS. 5-8).

### 5.4.4 Partial Viral Genome

In preferred embodiments, landing pad polynucleotides (e.g., a landing pad plasmid or integrated into a cell) described herein comprise a partial viral genome. The partial viral genome can be naturally occurring or a variant of a naturally occurring partial viral genome.

The partial viral genome can be derived from be any virus whose genome can be activated (e.g., upon reconstitution in vivo). Exemplary viruses include, e.g., retroviruses (e.g., lentiviruses (e.g., HIV)), adenoviruses, parvoviruses (e.g., adeno-associated virus), and viruses of family orthoherpesviridae viruses (e.g., herpes viruses, e.g., herpes simplex virus).

In some embodiments, the partial viral genome is a partial retrovirus genome. In some embodiments, the partial virus retrovirus genome is a partial lentivirus genome (e.g., a partial HIV genome). In some embodiments, the partial virus retrovirus genome is a partial HIV genome. In some embodiments, the partial virus genome is a partial adenovirus genome. In some embodiments, the partial viral genome is a partial parvovirus virus genome. In some embodiments, the partial virus genome is a partial adeno-associated virus genome. In some embodiments, the partial viral genome is a partial genome from a virus from the orthoherpesviridae family. In some embodiments, the partial viral genome is a partial herpes virus genome. In some embodiments, the partial viral genome is a partial herpes simplex virus genome.

In some embodiments, the partial viral genome comprises or consists of one or more viral long terminal repeat (LTR). In some embodiments, the partial virus genome in a landing pad of the disclosure has only one LTR (see, e.g., FIG. 5). In some embodiments, the partial virus genome in a landing pad of the disclosure has two LTRs (e.g., a 5′ LTR and 3′ LTR, for example, when the transfer polynucleotide (e.g., transfer plasmid) (e.g., of the same system) lacks an LTR) (see, e.g., FIG. 6).

In some embodiments, the partial viral genome comprises a 5′ LTR. In some embodiments, the partial viral genome comprises a 3′ LTR. In some embodiments, the partial viral genome comprises a 5′ LTR and a 3′ LTR. In some embodiments, the partial viral genome comprises a 5′ LTR and lacks a 3′ LTR. In some embodiments, the partial viral genome comprises a 3′ LTR and lacks a 5′ LTR. In some embodiments, the partial viral genome consists of a 5′ LTR. In some embodiments, the partial viral genome consists of a 3′ LTR. In some embodiments, the partial viral genome consists of a 5′ LTR and lacks a 3′ LTR. In some embodiments, the partial viral genome consists of a 3′ LTR and lacks a 5′ LTR.

In embodiments, wherein the partial viral genome contains a 3′ LTR, the 3′ LTR can be a reference 3′ LTR (e.g., wild type) or a variant thereof. In some embodiments, the 3′ LTR comprises a full length U3 region (i.e., the 3′ LTR does not have a deletion of any part of the U3 region). In some embodiments, the 3′ LTR comprises a portion of U3 region (i.e., the 3′ LTR has a deletion of part of the U3 region). In some embodiments, the 3′ LTR comprises a functional deletion of at least a portion of the U3 region (i.e., the 3′ LTR has a deletion of at least a portion of the U3 region to render it non-functional). In some embodiments, the 3′ LTR does not contain a U3 region (i.e., the 3′ LTR has a deletion of the entire U3 region). In some embodiments, the comprises a functional variation (e.g., one or more nucleotide substitution) of at least a portion of the U3 region (i.e., the 3′ LTR has a deletion of at least a portion of the U3 region to render it non-functional).

The partial viral genome can include additional genes encoding one or more viral proteins. For example, the partial viral genome can also include one or more viral structural gene, regulatory gene, and/or accessory gene. For example, in embodiments wherein the partial viral genome is an HIV partial viral genome, the partial viral genome of the landing pad polynucleotide may comprise any one or more HIV virus structural or polymerase genes (e.g., gag, pol, env genes), HIV virus regulatory genes (e.g., tat, rev genes), and/or HIV virus accessory genes (e.g., HIV-1 vif, vpr, vpu, nef genes). In some embodiments, the partial viral genome comprises one or more viral regulatory elements. For examples, in embodiments wherein the partial viral genome is an HIV partial viral genome, the partial viral genome of the landing pad polynucleotide may comprise any one or more HIV regulatory elements (e.g., ψ, RRE, cPPT, CTS).

As described elsewhere herein, in embodiments, wherein the landing pad (e.g., in a system described herein or integrated into a cell described herein) comprises a partial viral genome; the corresponding transfer polynucleotide (e.g., that are part of the same system) can comprise a corresponding part of the same partial viral genome. As such, integration of the transfer polynucleotide (e.g., transfer plasmid) at the recombinase recognition site in the recombinase landing pad results in a reconstituted, or reconstructed, viral genome (e.g., comprising two LTRs, viral protein genes, viral regulatory genes and/or viral accessory genes). The partial viral genome in the landing pad is preferably from the same type of virus as the partial viral genome in a corresponding transfer polynucleotide (e.g., transfer plasmid) (e.g., that are part of the same system) that is to be integrated into the landing pad.

For example, in embodiments, wherein the landing pad (e.g., in a system described herein or integrated into a cell described herein) described herein comprises a partial viral genome comprising a 5′ LTR (or a variant, fragment, or component thereof); the corresponding transfer polynucleotide may comprise a partial viral genome from the corresponding 3′ LTR (or a variant, fragment, or component thereof) of the same viral genome. For example, in some embodiments, the landing pad (e.g., in a system described herein or integrated into a cell described herein) described herein comprises a partial HIV viral genome comprising an HIV 5′ LTR (or a variant, fragment, or component thereof); and the corresponding transfer polynucleotide comprises a partial viral genome comprising the corresponding HIV 3′ LTR (or a variant, fragment, or component thereof).

### 5.4.5 Selectable Marker Genes

In preferred embodiments, the landing pad polynucleotide (e.g., a landing pad plasmid or integrated into a cell) comprises one or more (e.g., 1, 2, or 3, or more) selectable marker genes. The one or more selectable marker genes can be utilized for positive selection of landing polynucleotides that have integrated into the DNA of a cell.

Various selectable marker genes are known in the art and a person of ordinary skill in the art can select one or more suitable selectable marker gene for use in a transfer polynucleotide (e.g., transfer plasmid) described herein. Exemplary selectable marker genes, include, but are not limited to drug resistance genes (e.g., antibiotic resistance genes (e.g., puromycin resistance genes, ampicillin resistance genes, gentamycin resistance genes, streptomycin resistance genes, kanamycin resistance genes, hygromycin resistance genes, cefoxitin resistance genes, amoxicillin resistance genes, tetracycline resistance genes, sulfadiazine resistance genes, chloramphenicol resistance genes, fosfomycin resistance genes, trimethoprim resistance genes, erythromycin resistance genes, rifampicin resistance genes, azithromycin resistance genes, Blasticidin resistance genes)); detectable proteins (e.g., fluorescent proteins (e.g., Green Fluorescent Protein (GFP), Blue Fluorescent Protein (BFP), yellow fluorescent protein (YFP), cyan fluorescent protein (CFP), red fluorescent protein (RFP), Zs Green)); suicide genes (e.g., Herpes simplex virus thymidine kinase (HSV-TK) gene, human inducible caspase 9 (iCasp9) gene, mutant human thymidylate kinase (mTMPK) gene, human CD20 gene).

In some embodiments, the landing pad polynucleotide (e.g., a landing pad plasmid or integrated into a cell) comprises at least one an antibiotic resistance gene. In some embodiments, the landing pad polynucleotide comprises a gene encoding a detectable protein. In some embodiments, the detectable protein is a fluorescent protein.

In some embodiments, the landing pad polynucleotide comprises more than 1 (e.g., 2, 3, 4, 5, or more) selectable marker gene. In some embodiments, the landing pad polynucleotide comprises a plurality of selectable marker gene. In some embodiments, at least 2 of the selectable marker genes in the plurality are different types (e.g., on is an antibiotic resistance gene and one encodes a detectable protein). In some embodiments, the landing pad polynucleotide comprises at least one antibiotic resistance gene and at least one gene encoding a detectable protein. In some embodiments, the landing pad polynucleotide comprises at least one suicide gene (e.g., Herpes simplex virus thymidine kinase (HSV-TK) gene, human inducible caspase 9 (iCasp9) gene, mutant human thymidylate kinase (mTMPK) gene, human CD20 gene).

In some embodiments, any selectable marker genes within a landing pad polynucleotide (e.g., a landing pad plasmid or integrated into a cell) are different from any selectable marker genes within a transfer polynucleotide (e.g., transfer plasmid) described herein (e.g., that are part of a system described herein). As such, the integration of a landing pad into the genomic DNA of a cell could be selected for separately from the integration of a transfer polynucleotide described herein into an integrated landing pad.

In some embodiments, the selectable marker gene(s) in the landing pad (e.g., landing pad plasmid) are only transcriptionally active when there has been no integration of a transfer polynucleotide of the disclosure into the landing pad.

### 5.4.6 Gene Regulatory Elements

In preferred embodiments, the landing pad polynucleotide (e.g., a landing pad plasmid or integrated into a cell) comprises one or more (e.g., 1, 2, 3, 4, 5, or more) gene regulatory elements.

Exemplary gene regulatory elements include, but are not limited to, e.g., promoters, enhancers, internal ribosome entry sites (IRESs), 2A sequences, viral posttranscriptional regulatory elements (e.g., WPRE), transcription termination sequences (e.g., SV40, hGH, BGH, rbGlob terminators), and polyadenylation signal sequences (e.g., polyA sequence).

In some embodiments, the landing pad polynucleotide comprises one or more of a promoter; an enhancer; an IRES; a viral posttranscriptional regulatory element (e.g., WPRE); a transcription termination sequence (e.g., SV40, hGH, BGH, rbGlob terminators); a polyadenylation signal sequence (e.g., polyA sequence); and/or a polynucleotide sequence encoding a cleavable peptide, such as self-cleaving peptides (e.g., 2A peptides, e.g., T2A, P2A, E2A, or F2A peptides); an rtTa element encoding tetR+VP16 fusion for activation of inducible expression; or any combination of the foregoing.

In some embodiments, the landing pad polynucleotide comprises a promoter. In some embodiments, the landing pad polynucleotide comprises an enhancer. In some embodiments, the landing pad polynucleotide comprises an IRES. In some embodiments, the landing pad polynucleotide comprises a polyA. In some embodiments, the landing pad polynucleotide comprises a viral posttranscriptional regulatory element. In some embodiments, the viral posttranscriptional regulatory element is a Woodchuck Hepatitis Virus Posttranscriptional Regulatory Element (WPRE). In some embodiments, the landing pad polynucleotide comprises a transcription termination sequence (e.g., (SV40, hGH, BGH, rbGlob terminators). In some embodiments, the landing pad polynucleotide comprises a polyadenylation signal sequence (e.g., polyA sequence). In some embodiments, the landing pad polynucleotide comprises a polynucleotide sequence encoding a cleavable peptide, such as self-cleaving peptides (e.g., 2A peptides, e.g., T2A, P2A, E2A, or F2A peptides). 2A peptides are typically positioned between protein coding polynucleotide sequences in order to induce ribosomal skipping during translation.

In certain embodiments, the landing pad polynucleotide (e.g., landing pad plasmid) comprises an IRES operably connected to one or more selectable marker genes (e.g., described herein). In certain embodiments, the landing pad polynucleotide (e.g., landing pad plasmid) comprises a plurality of selectable maker genes (e.g., described herein), wherein each selectable marker gene of the plurality is separated by a 2A element (e.g., a T2A, P2A, E2A, or F2A element).

In some embodiments, the landing pad polynucleotide (e.g., landing pad plasmid) comprises at least one promoter. In some embodiments, the landing pad polynucleotide (e.g., landing pad plasmid) comprises at least one enhancer. In some embodiments, the landing pad polynucleotide (e.g., landing pad plasmid) comprises at least one promoter and at least one enhancer.

Suitable promoters are known in the art and can be selected by a person of ordinary skill in the art. Promoters can be constitutive, inducible, and/or repressible. In some embodiments, the promoter is a constitutive promoter (e.g., a CMV promoter). In some embodiments, the landing pad polynucleotide (e.g., landing pad plasmid) comprises at least one inducible promoter. For example, an antibiotic inducible promoter (e.g., a doxycycline inducible promoter (e.g., PTRE3GS)). In some embodiments, the landing pad polynucleotide (e.g., landing pad plasmid) comprises at least one constitutive promoter. In some embodiments, the landing pad polynucleotide (e.g., landing pad plasmid) comprises at least one repressible promoter. In some embodiments, the landing pad polynucleotide (e.g., landing pad plasmid) comprises at least one constitutive promoter and at least on inducible promoter. In some embodiments, the landing pad polynucleotide (e.g., landing pad plasmid) comprises at least one constitutive promoter and at least on repressible promoter.

In particular embodiments, an inducible promoter (e.g., PTRE3GS) is operably linked to a recombinase recognition site in the landing pad polynucleotide. As such, upon integration of a corresponding transfer polynucleotide (e.g., part of the same system) the polynucleotide sequence encoding the protein of interest (e.g., the viral entry protein of interest) will be operably linked to the inducible promoter. In particular embodiments, a constitutive promoter (e.g., a CMV promoter) is operably linked to a polynucleotide encoding a recombinase (e.g., Bxb1) in the landing pad polynucleotide.

In particular embodiments, a repressible promoter (e.g., PTRE3GS) is operably linked to a recombinase recognition site in the landing pad polynucleotide. As such, upon integration of a corresponding transfer polynucleotide (e.g., part of the same system) the polynucleotide sequence encoding the protein of interest (e.g., the viral entry protein of interest) will be operably linked to the repressible promoter. In particular embodiments, a constitutive promoter (e.g., a CMV promoter) is operably linked to a polynucleotide encoding a recombinase (e.g., Bxb1) in the landing pad polynucleotide.

### 5.4.7 Homology Arms for Site-Specific Integration

As described herein, the landing pad polynucleotides can be designed for site-specific integration into a cellular genome. In some embodiments, the landing pad comprises one or more (e.g., 2) homology arms to mediate site-specific insertion using a genetic engineered system such as, CRISPR-Cas (see, e.g., § 5.5.3). As such, in some embodiments, the landing pad plasmid comprises a right homology arm and a left homology arm flanking the landing pad to be integrated into the cellular genome.

For example, in some embodiments, an HDR (homology directed repair) CRISPR-Cas system can be utilized, wherein the molecular machinery of the cell will utilize the landing pad polynucleotide as a donor template nucleic acid molecule in repairing and/or resolving a cleavage site in the cellular genome mediated by a Cas endonuclease (or functional fragment, functional variant, or domain thereof), wherein landing pad donor sequence will be incorporated into the target site of the cellular genome through e.g., HDR. See, e.g., U.S. Pat. No. 8,697,359, the entire contents of which is incorporated herein by reference for all purposes. For such methods, the landing pad plasmid may comprise a right homology arm and a left homology arm flanking the landing pad to be integrated into the cellular genome.

In some embodiments, the homology arms are directed to a safe harbor locus. Exemplary safe harbor loci in human cells include, but are not limited to, AAVS1, CCR5, Rosa26, and H11. In some preferred embodiments, the homology arms are directed to the AAVS1 gene. In some embodiments, the homology arms are directed to the CCR5 gene. In some embodiments, the homology arms are directed to the Rosa26 gene. In some embodiments, the homology arms are directed to the H11 gene.

### 5.4.8 Integrated Landing Pads

As described above, the landing pad polynucleotides described herein (see, e.g., § 5.4) can be isolated (e.g., not integrated in genomic DNA) (e.g., a landing pad plasmid) or integrated in a cell's genomic DNA (e.g., a landing pad).

In some embodiments, the landing pad polynucleotide is isolated.

In some embodiments, the landing pad polynucleotide is integrated into a cell's genomic DNA. In preferred embodiments, the integration of the landing pad into the genome of the cell is irreversible.

A person of ordinary skill in the art would understand that introduction of e.g., a landing pad plasmid (e.g., described herein) and subsequent integration may result in only a portion of an isolated landing pad plasmid being integrated into the genomic DNA of the cell. The portion of the landing pad plasmid (generally referred to herein as the landing pad) would be integrated into the genomic DNA which contains all of the elements that make up the subject landing pad.

### 5.5 Cells Comprising Integrated Landing Pads

Provided herein are, inter alia, cells (e.g., a cell or a population of cells) comprising a landing pad polynucleotide described herein integrated into the cell's genome/genomic DNA. For example, a landing pad polynucleotide described in § 5.4.

In preferred embodiments, the cells comprise a landing pad polynucleotide irreversibly integrated into the genome of the cells. In preferred embodiments, the cells are in vitro. In some embodiments, the cells are ex vivo.

### 5.5.1 Cell Types

The cell can be any type of cell that supports virus (e.g., lentivirus) production. In some embodiments, the cell is a mammalian cell or a mammalian cell line. In preferred embodiments, the cell is a human cell. In some embodiments, the cell is an animal cell. In some embodiments, the cell is a mouse, rat, hamster, rabbit, cat, dog, or non-human primate cell.

Exemplary cell lines include, but are not limited to, human embryonic kidney (HEK) e.g., HEK293, HEK 293F), HeLa, SH-SY5Y, MCF-7, H1, H9, CHO, COS, PC3, Vero, MC3T3, NSO, VERY, BHK, MDCK, W138, BT483, Hs578T, HTB2, BT20, T47D), CRL7030, and HsS78Bst cells. In some embodiments, the cell is a (HEK) e.g., HEK293, HEK 293F), HeLa, SH-SY5Y, MCF-7, H1, or H9 cell. In some embodiments, the cell is a HEK cell or cell line (e.g., HEK293 cells, HEK 293F cells, HEK 293 FT cells, HEK 293T cells, HEK 293S cells, HEK 293FTM cells, HEK 293SG cells, HEK 293SGGD cells, HEK 293H cells, HEK 293E cells, HEK EBNA1-6E cells, HEK 293MSR cells, HEK 293A cells). In some embodiments, the cells are HEK 293T cells.

### 5.5.2 Locus and Copy Number

In some embodiments, the landing pad polynucleotide is integrated into a safe harbor genomic locus. Exemplary safe harbor loci in human cells include, but are not limited to, AAVS1, CCR5, Rosa26, and H11. In some embodiments, the landing pad is integrated into the AAVS1 gene locus. In some embodiments, the landing pad is integrated into the CCR5 gene locus. In some embodiments, the landing pad is integrated into the Rosa26 gene locus. In some embodiments, the landing pad is integrated into the H1l gene locus.

In preferred embodiments, the cell(s) comprise a single recombinase landing pad integrated at a single genomic locus in the cell(s). In preferred embodiments, the cell(s) comprise a single recombinase landing pad integrated at a single genomic locus in a single chromosome in the cell(s). Validation of single copy insertion can be determined using standard methods known in the art, including, e.g., inverse PCR and genotyping PCR, flow cytometry, Sanger sequencing, and Southern blotting. See, e.g., Maes, Stefanie et al. “Deep mutational scanning of proteins in mammalian cells.” Cell reports methods vol. 3, 11 (2023): 100641. doi: 10.1016/j.crmeth.2023.100641—and references cited therein; the entire contents of each of which are incorporated herein by reference for all purposes.

### 5.5.3 Methods of Site Specific Landing Pad Integration

Methods of integrating landing pad polynucleotides into cells are known in the art. See, e.g., Maes, Stefanie et al. “Deep mutational scanning of proteins in mammalian cells.” Cell reports methods vol. 3, 11 (2023): 100641. doi: 10.1016/j.crmeth.2023.100641; Hirano N., Muroi T., Takahashi H., Haruki M. Site-specific recombinases as tools for heterologous gene integration. Appl. Microbiol. Biotechnol. 2011; 92:227-239. doi: 10.1007/s00253-011-3519-5; Xu Z., Thomas L., Davies B., Chalmers R., Smith M., Brown W. Accuracy and efficiency define Bxb1 integrase as the best of fifteen candidate serine recombinases for the integration of DNA into the human genomc. BMC Biotechnol. 2013; 13:87-103. doi: 10.1186/1472-6750-13-87; Jones, Eric M et al. “Structural and functional characterization of G protein-coupled receptors with deep mutational scanning.” eLife vol. 9 e54895. 21 Oct. 2020, doi: 10.7554/eLife.54895; Chong, Rockie et al. “A Multiplexed Assay for Exon Recognition Reveals that an Unappreciated Fraction of Rare Genetic Variants Cause Large-Effect Splicing Disruptions.” Molecular cell vol. 73, 1 (2019): 183-194.c8. doi: 10.1016/j.molcel.2018.10.037; Matreyek, Kenneth A et al. “A platform for functional assessment of large variant libraries in mammalian cells.” Nucleic acids research vol. 45, 11 (2017): c102. doi: 10.1093/nar/gkx183; Shin, Seunghyeon et al. “Comprehensive Analysis of Genomic Safe Harbors as Target Sites for Stable Expression of the Heterologous Gene in HEK293 Cells.” ACS synthetic biology vol. 9, 6 (2020): 1263-1269. doi: 10.1021/acssynbio.0c00097; the entire contents of each of which are incorporated herein by reference for all purposes.

For example, landing pads can be integrated into the genome of the cell(s) in a site-specific manner using known methods in the art. For example, via homologous recombination induced via genome-editing methods such as CRISPR-Cas, zinc-finger nucleases (ZFNs), transcription activator-like effector nucleases (TALENs), and engineered meganucleases.

General information on CRISPR/Cas systems, components thereof, testing, and delivery of such components, including e.g., methods, materials, delivery vehicles, vectors, particles, AAV, and methods of making and using thereof, including as to amounts and formulations can be found in the art, see, e.g., Makarova et al. (2018) The CRISPR Journal 1 (5): 325-336; and Adli (2018) Nat. Communications 9:1911, Silva 2011, Makarova K S, Koonin E V. Annotation and Classification of CRISPR-Cas Systems. Methods Mol Biol. 2015; 1311:47-75. doi: 10.1007/978-1-4939-2687-9_4; WO2014093595A1, WO2014093622A2, WO2014093635A1, WO2014093655A2, WO2014093661A2, WO2014093694A1, WO2014093701A1, WO2014093709A1, WO2014093712A1, WO2014093718A1, WO2014204723A1, WO2014204724A1, WO2014204725A1, WO2014204726A1, WO2014204727A1, WO2014204728A1, WO2014204729A1, WO2020047124A1, WO2021178709A1, WO2021178717A2, WO2021178720A2, WO2021248102A1, the entire contents of each of which is incorporated by reference herein in their entirety for all purposes.

In some embodiments, for example HDR (homology directed repair) CRISPR-Cas system can be utilized, wherein the molecular machinery of the cell will utilize the landing pad polynucleotide as a donor template nucleic acid molecule in repairing and/or resolving a cleavage site in the cellular genome mediated by a Cas endonuclease (or functional fragment, functional variant, or domain thereof), wherein landing pad donor sequence will be incorporated into the target site of the cellular genome through e.g., HDR. See, e.g., U.S. Pat. No. 8,697,359, the entire contents of which is incorporated herein by reference for all purposes.

General information on ZFN genetic engineering systems, components thereof, and delivery of such components, including e.g., methods, materials, delivery vehicles, vectors, particles, and methods of making and using can be found in the art, see, e.g., Porteus and Baltimore (2003) Science 300:763; Miller et al. (2007) Nat. Biotechnol. 25:778-785; Sander et al. (2011) Nature Methods 8:67-69; and Wood et al. (2011) Science 333:307, Silva 2011, the full contents of each of which is incorporated by reference herein in their entirety for all purposes.

General information on TALEN genetic engineering systems, components thereof, testing, and delivery of such components, including e.g., methods, materials, delivery vehicles, vectors, particles, and methods of making and using can be found in the art, see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30 (5): 460-465, Silva 2011, the full contents of each of which is incorporated by reference herein in their entirety for all purposes.

General information on meganuclease genetic engineering systems, components thereof, testing, and delivery of such components, including e.g., methods, materials, delivery vehicles, vectors, particles, and methods of making and using can be found in the art, see, e.g., Silva 2011, the full contents of each of which is incorporated by reference herein in their entirety for all purposes.

### 5.6 Cell Libraries (e.g., Encoding Viral Entry Proteins)

Also provided herein are, inter alia, cell libraries (e.g., collections) comprising a plurality of cells each encoding a protein of interest (e.g., a viral entry protein). Each of the cells in the library comprises an integrated landing pad polynucleotide described herein, and an integrated transfer polynucleotide described herein encoding a protein of interest (e.g., a viral entry protein). For example, FIG. 3B (middle) shows a library of cells, each cell comprising an integrated landing pad and an integrated transfer polynucleotide, wherein each transfer polynucleotide encodes a different viral entry protein. These cell libraries, may also be referred to herein as libraries of cell-stored proteins or cell-stored protein libraries.

In some embodiments, each of the cells within the plurality encodes a different protein of interest (e.g., a different viral entry protein) relative to the other cells in the plurality.

In some embodiments, the library comprises a plurality of cells, each encoding a different protein. In some embodiments, the library comprises (a) a plurality of cells, each encoding a different variant of a reference protein; and optionally (b) a cell encoding the reference protein. In some embodiments, the library comprises (a) a plurality of cells, each encoding a different variant of a reference protein; and (b) a cell encoding the reference protein.

The reference protein can be any peptide or protein (e.g., an enzyme, a structural protein, a targeting protein, a signaling protein, an antibody or antigen-binding fragment of an antibody). For example, any protein of interest described herein (see, e.g., §§ 5.2.3, 5.2.3.1). In some embodiments, the reference protein is a protein of interest described in §§ 5.2.3 (e.g., § 5.2.3.1).

In some embodiments, the reference protein is a non-viral protein (e.g., a cell targeting protein or peptide, e.g., an antibody (e.g., a scFv, a Fab)).

In some embodiments, the reference protein is a viral protein. In preferred embodiments, the viral protein is a viral entry protein (e.g., described herein, see, e.g., § 5.2.3.1) (e.g., a spike protein of a SARS virus (e.g., a SARS-COV-2 virus); an HA protein of an influenza virus). In certain embodiments, the viral entry protein is a viral entry protein described in § 5.2.3.1. In certain embodiments, the viral entry protein is a SARS-COV-2 spike protein. In certain embodiments, the viral entry protein is an influenza HA protein.

In some embodiments, each of the cells within the plurality encodes a different viral entry protein relative to the other cells in the plurality.

In some embodiments, the library comprises a plurality of cells, each encoding a different viral entry protein. In some embodiments, the library comprises (a) a plurality of cells, each encoding a different variant of a reference viral entry protein; and optionally (b) a cell encoding the reference viral entry protein. In some embodiments, the library comprises (a) a plurality of cells, each encoding a different variant of a reference viral entry protein; and (b) a cell encoding the reference viral entry protein.

In some embodiments, other than the polynucleotide sequence encoding the protein variant, the sequence of the exogenous DNA (i.e., the integrated landing pad and integrated transfer polynucleotide) in the plurality is substantially identical. In some embodiments, the sequence of the exogenous DNA (i.e., the integrated landing pad and integrated transfer polynucleotide) in the plurality is substantially identical outside of the polynucleotide sequence encoding the protein variant. In some embodiments, other than the polynucleotide sequence encoding the protein variant, the sequence of the exogenous DNA (i.e., the integrated landing pad and integrated transfer polynucleotide) in the plurality is identical. In some embodiments, the sequence of the exogenous DNA (i.e., the integrated landing pad and integrated transfer polynucleotide) in the plurality is identical outside of the polynucleotide sequence encoding the protein variant. In some embodiments, other than the polynucleotide sequence encoding the protein variant, the sequence of the exogenous DNA (i.e., the integrated landing pad and integrated transfer polynucleotide) in the plurality is at least 95%, 96%, 97%, 98%, 99% or 100% identical. In some embodiments, the sequence of the exogenous DNA (i.e., the integrated landing pad and integrated transfer polynucleotide) in the plurality is at least 95%, 96%, 97%, 98%, 99% or 100% identical outside of the polynucleotide sequence encoding the protein variant.

In some embodiments, the plurality comprises at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or more different cells (i.e., encoding a different protein variant or reference protein). In some embodiments, the plurality comprises more than 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 or more different cells (i.e., encoding a different protein variant or reference protein).

In some embodiments, the cell library is made by a method described herein (see, e.g., § 5.14.1). In some embodiments, the cell library is made by a method described in § 5.14.1.

Provided herein are cell libraries made by a method described herein (see, e.g., § 5.14.1).

### 5.7 Virion Libraries

Also provided herein are, inter alia, virion libraries (e.g., collections) comprising a plurality of virions, each virion of the plurality expressing a protein of interest (e.g., a viral entry protein) on the surface and encoding the same protein of interest (e.g., viral entry protein) within the genome of the virion (phenotype-genotype linked). In some embodiments, the library comprises a plurality of virions, each virion of the plurality expressing a different protein of interest (e.g., a different viral entry protein) on the surface and encoding the same protein of interest (e.g., viral entry protein) within the genome of the virion (phenotype-genotype linked).

In some embodiments, each of the virions in the plurality expresses (and encodes) a different protein variant (e.g., viral entry protein variant). For example, FIG. 3B (right) shows a plurality of virions each virion expressing a different viral entry protein on the surface and encoding the same viral entry protein within the genome of the virion.

In some embodiments, the library comprises a plurality of virions, each virion of the plurality expressing a viral entry protein on the surface and encoding the same viral entry protein within the genome of the virion (phenotype-genotype linked). In some embodiments, the library comprises a plurality of virions, each virion of the plurality expressing a different viral entry protein on the surface and encoding the same viral entry protein within the genome of the virion (phenotype-genotype linked).

In some embodiments, the library comprises (a) a plurality of virions, each virion of the plurality expressing a variant of a reference viral entry protein on the surface and encoding the same variant viral entry protein within the genome of the virion (phenotype-genotype linked); and optionally (b) a virion expressing the reference viral entry protein on the surface and encoding the same reference viral entry protein within the genome of the virion (phenotype-genotype linked).

In some embodiments, the library comprises (a) a plurality of virions, each virion of the plurality expressing a variant of a reference viral entry protein on the surface and encoding the same variant viral entry protein within the genome of the virion (phenotype-genotype linked); and (b) a virion expressing the reference viral entry protein on the surface and encoding the same reference viral entry protein within the genome of the virion (phenotype-genotype linked).

The reference protein can be any peptide or protein (e.g., an enzyme, a structural protein, a targeting protein, a signaling protein, an antibody or antigen-binding fragment of an antibody). For example, any protein of interest described herein (see, e.g., §§ 5.2.3, 5.2.3.1). In some embodiments, the reference protein is a protein of interest described in §§ 5.2.3, 5.2.3.1.

In some embodiments, the reference protein is a non-viral protein (e.g., a cell targeting protein or peptide, e.g., an antibody (e.g., a scFv, a Fab)).

In some embodiments, the reference protein is a viral protein. In certain embodiments, the viral protein is a viral entry protein (e.g., described herein, see, e.g., § 5.2.3.1) (e.g., a spike protein of a SARS virus (e.g., a SARS-COV-2 virus); an HA protein of an influenza virus). In certain embodiments, the viral entry protein is a viral entry protein described in § 5.2.3.1. In certain embodiments, the viral entry protein is a SARS-COV-2 spike protein. In certain embodiments, the viral entry protein is an influenza HA protein.

In some embodiments, the plurality (e.g., library, collection) comprises at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or more different virions (i.e., expressing/encoding a different protein variant or reference protein). In some embodiments, the plurality (e.g., library, collection) comprises more than 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 or more different cells (i.e., expressing/encoding a different protein variant or reference protein).

In some embodiments, the virions are retrovirus based (e.g., lentiviruses (e.g., HIV)), adenovirus based, parvovirus (e.g., adeno-associated virus) based, or virus based of family orthoherpesviridae viruses (e.g., herpes viruses, e.g., herpes simplex virus). In some embodiments, the virions are retrovirus based. In some embodiments, the virions are lentivirus based. In some embodiments, the virions are HIV based. In some embodiments, the virions are adenovirus based. In some embodiments, the virions are parvovirus based. In some embodiments, the virions are adeno-associated virus based. In some embodiments, the virions are orthoherpesviridae family based. In some embodiments, the virions are herpes virus based. In some embodiments, the virions are herpes simplex virus based.

In some embodiments, the virion library is made by a method described herein (see, e.g., § 5.14.2). In some embodiments, the virion library is made by a described in § 5.14.2.

Provided herein are virion libraries (e.g., collections) made by a method described herein (e.g., made by a method described in § 5.14.2).

### 5.8 Polynucleotides

Various polynucleotides are provided herein, including, e.g., transfer polynucleotides, transfer plasmids, landing pad polynucleotides, landing pad plasmids, etc. (see, e.g., §§ 5.2, 5.4).

Any of the polynucleotides described herein can be double-stranded or single-stranded. Any of the polynucleotides described herein can be linear or circular. Any of the polynucleotides described herein can comprise DNA nucleotides, RNA nucleotides, and/or non-natural nucleotides.

Any portion or entirety of any polynucleotide described herein can be codon optimized (e.g., a protein coding polynucleotide sequence) can be codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

Any of the polynucleotides described herein (e.g., transfer polynucleotides, transfer plasmids, landing pad polynucleotides, landing pad plasmids, etc.) can be produced recombinantly or synthetically, using standard reagents, techniques, and methods that are well known to those of ordinary skill in the art.

### 5.9 Vectors

Any of the polynucleotides described herein (e.g., transfer polynucleotides, landing pad polynucleotides (see, e.g., §§ 5.2, 5.4)) can be incorporated into a vector. As such, provided herein are, inter alia, vectors comprising any one or more polynucleotide described herein (e.g., transfer polynucleotides, landing pad polynucleotides).

In some embodiments, the vector is a non-viral vector. In preferred embodiments, the vector is a plasmid. A person of ordinary skill in the art is aware of suitable plasmids (e.g., commercially available plasmids) as well as methods of preparation.

In some embodiments, the vector is a viral vector. A person of ordinary skill in the art is aware of suitable viral vector (e.g., commercially available viral vectors) as well as methods of preparation.

### 5.10 Cells

Provided herein are, inter alia, cells comprising any one or more of a transfer polynucleotide (e.g., transfer plasmid described herein); a landing pad polynucleotide (e.g., landing pad plasmid) described herein; a landing pad described herein integrated into the cell's genome; a landing pad described herein integrated into the cell's genome and a transfer polynucleotide (e.g., transfer plasmid) described herein (e.g., not integrated into the cell's genome); and/or a landing pad described herein integrated into the cell's genome and a transfer polynucleotide described herein integrated into the landing pad in the cell's genome.

As such, provided herein are, inter alia, cells comprising a transfer polynucleotide (e.g., transfer plasmid described herein). Also provided herein are, inter alia, cells comprising a landing pad polynucleotide (e.g., landing pad plasmid) described herein. Also provided herein are, inter alia, cells comprising a landing pad described herein integrated into the cells genome. Also provided herein are, inter alia, cells comprising a landing pad described herein integrated into the cell's genome and a transfer polynucleotide (e.g., transfer plasmid) described herein (e.g., not integrated into the cell's genome). Also provided herein are, inter alia, cells comprising a landing pad described herein integrated into the cell's genome and a transfer polynucleotide described herein integrated into the landing pad in the cell's genome.

In some embodiments, the cells are in vitro. In some preferred embodiments, the cells are in vitro. In some embodiments, the cells are ex vivo.

In some embodiments, the cell is a mammalian cell or a mammalian cell line. In specific embodiments, the cell is a human cell. In some embodiments, the cell is a mammalian cell. In some embodiments, the cell is an animal cell. In some embodiments, the cell is a mouse, rat, hamster, rabbit, cat, dog, or non-human primate cell.

Exemplary cell lines include, but are not limited to, human embryonic kidney (HEK) e.g., HEK293, HEK 293F), HeLa, SH-SY5Y, MCF-7, H1, H9, CHO, COS, PC3, Vero, MC3T3, NSO, VERY, BHK, MDCK, W138, BT483, Hs578T, HTB2, BT20, T47D), CRL7030, and HsS78Bst cells. In some embodiments, the cell is a (HEK) e.g., HEK293, HEK 293F), HeLa, SH-SY5Y, MCF-7, H1, or H9 cell. In some embodiments, the cell is a HEK cell or cell line (e.g., HEK293 cells, HEK 293F cells, HEK 293 FT cells, HEK 293T cells, HEK 293S cells, HEK 293FTM cells, HEK 293SG cells, HEK 293SGGD cells, HEK 293H cells, HEK 293E cells, HEK EBNA1-6E cells, HEK 293MSR cells, HEK 293A cells). In some embodiments, the cells are HEK 293T cells.

### 5.11 Systems

Provided herein are, inter alia, systems comprising any one or more of a transfer polynucleotide (e.g., transfer plasmid) described herein (see, e.g., § 5.2); a landing pad polynucleotide (e.g., landing pad plasmid) described herein (see, e.g., § 5.4); a cell (or population of cells) comprising a landing pad plasmid integrated into the cell's genome described herein (see, e.g., § 5.5); a library of transfer polynucleotides (e.g., transfer plasmids) described herein (see, e.g., § 5.3); a cell library described herein (see, e.g., § 5.6); a cell library made by a method described herein (see, e.g., § 5.14.1); a library of virions expressing and encoding a protein of interest (e.g., a viral entry protein) described herein (see, e.g., § 5.7); and/or a virion library made by a method described herein (see, e.g., § 5.14.2).

Any of the systems described herein may be used in any of the methods described herein (see, e.g., § 5.14).

### 5.11.1 Exemplary Systems

As such, provided herein are systems comprising (i) a transfer polynucleotide (e.g., a transfer plasmid) described herein (see, e.g., § 5.2) and (ii) a landing pad polynucleotide (e.g., a landing pad plasmid) described herein (see, e.g., § 5.4).

Also provided herein are systems comprising (i) a transfer polynucleotide (e.g., a transfer plasmid) described herein (see, e.g., § 5.2) and (ii) a cell comprising a landing pad described herein integrated into the cell's genome (see, e.g., § 5.5).

Also provided herein are systems comprising (i) a library of transfer polynucleotides (e.g., transfer plasmids) described herein (see, e.g., § 5.3) and (ii) a cell comprising a landing pad described herein integrated into the cell's genome (see, e.g., § 5.5).

Also provided herein are systems comprising (i) a cell library described herein (see, e.g., § 5.6) and (ii) a plurality of helper plasmids encoding one or more viral proteins sufficient for virion production in combination with the library (see, e.g., § 5.11.3.2).

Also provided herein are systems comprising (i) a cell library made by a method described herein (see, e.g., § 5.14.1) and (ii) a plurality of helper plasmids encoding one or more viral proteins sufficient for virion production in combination with the library (see, e.g., § 5.11.3.2).

Also provided herein are systems comprising (i) a library of virions expressing and encoding proteins (e.g., viral entry proteins) described herein (see, e.g., § 5.7); and (ii) a population of cells (see, e.g., § 5.10).

Also provided herein are systems comprising (i) a library of virions made by a method described herein (see, e.g., § 5.14.2); and (ii) a population of cells (see, e.g., § 5.10).

### 5.11.2 Complementary Elements

It will be clear to a person of ordinary skill in the art that where components of a system described herein comprise elements that correspond to one another (e.g., the recombinase recognition site of a transfer polynucleotide and the recombinase recognition site of the landing pad in the same system; the partial viral genomes, etc.), the elements should be complementary to be suitable for the function of the elements within the system.

Exemplary complementary elements include the recombinase recognition site of the transfer polynucleotide, the recombinase recognition sites of the landing pad polynucleotide, and the recombines (either encoded by the landing pad or provided exogenously). As such, the recombinase recognition site of the transfer polynucleotide and the recombinase recognition site of the landing pad polynucleotide should be complementary pairs, such that recombination can occur in the presence of a recombinase and under suitable conditions. For example, if the recombinase recognition site of the transfer polynucleotide is a Bxb1 attB site the recombinase recognition site of the landing pad polynucleotide may be a Bxb1 attP site. In addition, the recombinase of a system (whether encoded by the landing pad polynucleotide or provided separately within the system) should be complementary to (recognize) the recombinase recognition sites. For example, if the transfer polynucleotide comprises a Bxb1 attB site and the landing pad plasmid comprises a Bxb1 attP site, the recombinase may be a Bxb1 recombinase.

Other complementary elements include the partial viral genome of the transfer polynucleotide and the partial viral genome of the landing pad polynucleotide. For example, in embodiments, wherein the landing pad comprises a partial viral genome; the corresponding transfer polynucleotide can comprise a complementary part of the same partial viral genome. As such, integration of the transfer polynucleotide into the landing pad will result in a reconstituted, or reconstructed, viral genome (e.g., comprising two LTRs, viral protein genes, viral regulatory genes and/or viral accessory genes). In some embodiments, the partial viral genome in a landing pad is preferably from the same type of virus as the partial viral genome in a corresponding transfer polynucleotide that is to be integrated into the landing pad (in embodiments wherein the transfer polynucleotide comprises a partial viral genome).

For further example, in embodiments, wherein the landing pad polynucleotide comprises a partial viral genome comprising a 5′ LTR (or a variant, fragment, or component thereof); the corresponding transfer polynucleotide may comprise a partial viral genome from the corresponding 3′ LTR (or a variant, fragment, or component thereof) (e.g., of the same viral genome). For example, in some embodiments, the landing pad polynucleotide comprises a partial HIV viral genome comprising an HIV 5′ LTR (or a variant, fragment, or component thereof); and the corresponding transfer polynucleotide comprises a partial viral genome comprising the corresponding HIV 3′ LTR (or a variant, fragment, or component thereof).

Further the selectable makers of the transfer polynucleotide and the selectable markers of the landing pad polynucleotide, while not complementary, may be selected in a coordinated fashion such that none of the selectable markers utilized in the transfer plasmid are the same (or functionally the same (e.g., utilize the same selection agent)) as any of the selectable markers in the landing pad polynucleotide. As such, e.g., integration of the landing pad polynucleotide and integration of the transfer polynucleotide can be assessed individually without interference from the other.

### 5.11.3 Additional Components

### 5.11.3.1 Recombinases

In systems described herein comprising a landing pad, if the landing pad does not contain a polynucleotide sequence encoding a recombinase, a polynucleotide encoding a recombinase (e.g., a recombinase described herein) can further be part of the system. For example, in a system comprising (i) a transfer polynucleotide (e.g., a transfer plasmid) described herein and (ii) a cell comprising a landing pad described herein integrated into the cell's genome; if the landing pad does not contain a polynucleotide sequence encoding a recombinase, a polynucleotide encoding a recombinase (e.g., a recombinase described herein) can further be part of the system.

### 5.11.3.2 Helper Plasmids

In systems described herein comprising (i) a cell library encoding proteins of interest (e.g., viral entry proteins) described herein and (ii) a plurality of helper plasmids encoding one or more viral proteins sufficient for virion production in combination with the library; the required helper plasmids are known in the art. For example, helper plasmids for virion generation are described in e.g., Duvergé, Alexis, and Matteo Negroni. “Pseudotyping Lentiviral Vectors: When the Clothes Make the Virus.” Viruses vol. 12,11 1311. 16 Nov. 2020, doi: 10.3390/v12111311 (herein after “Duvergé”) and Merten, Otto-Wilhelm et al. “Production of lentiviral vectors.” Molecular therapy. Methods & clinical development vol. 3 16017. 13 Apr. 2016, doi: 10.1038/mtm.2016.17, the entire contents of each of which are incorporated herein by reference for all purposes.

For example, FIG. 3 of Duvergé outlines the helper plasmids of the first, second, and third generation vectors for virion production. As such, in some embodiments, the system comprises one or more an HIV based helper plasmid encoding any one or more of the HIV gag, pol, RRE, and rev proteins). Alternative viruses can be utilized for the pseudotyping backbone in addition to HIV, including e.g., vesicular stomatitis virus glycoprotein (VSV-G); and murine leukemia virus (MLV).

### 5.12 Compositions

In a one aspect, provided herein are compositions comprising any one or more of a transfer polynucleotide (e.g., transfer plasmid) described herein (see, e.g., § 5.2); a landing pad polynucleotide (e.g., landing pad plasmid) described herein (see, e.g., § 5.4); a cell (or population of cells) comprising a landing pad plasmid integrated into the cell's genome described herein (see, e.g., § 5.5); a library of transfer polynucleotides (e.g., transfer plasmids) described herein (see, e.g., § 5.3); a cell library described herein (see, e.g., § 5.6); a cell library made by a method described herein (see, e.g., § 5.14.1); a library of virions expressing and encoding a protein of interest (e.g., a viral entry protein) described herein (see, e.g., § 5.7); a virion library made by a method described herein (see, e.g., § 5.14.2); and/or a system described herein (see, e.g., § 5.11); or any combination of the foregoing.

In one aspect, provided herein are compositions comprising library of cells made by a method described herein (e.g., made by a method described in § 5.14.1). In some embodiments, the cells are mammalian cells line. In specific embodiments, the cells are human cells. In some embodiments, the cell are animal cells. In some embodiments, the cells are non-human mammal cells. In some embodiments, the cells are mouse, rat, hamster, rabbit, cat, dog, non-human mammal cells, or non-human primate cell. Exemplary cell lines include, but are not limited to, human embryonic kidney (HEK) e.g., HEK293, HEK 293F), HeLa, SH-SY5Y, MCF-7, H1, H9, CHO, COS, PC3, Vero, MC3T3, NSO, VERY, BHK, MDCK, W138, BT483, Hs578T, HTB2, BT20, T47D), CRL7030, and HsS78Bst cells. In some embodiments, the cell is a (HEK) e.g., HEK293, HEK 293F), HeLa, SH-SY5Y, MCF-7, H1, or H9 cell. In some embodiments, the cell is a HEK cell or cell line (e.g., HEK293 cells, HEK 293F cells, HEK 293 FT cells, HEK 293T cells, HEK 293S cells, HEK 293FTM cells, HEK 293SG cells, HEK 293SGGD cells, HEK 293H cells, HEK 293E cells, HEK EBNA1-6E cells, HEK 293MSR cells, HEK 293A cells). In some embodiments, the cells are HEK 293T cells.

In one aspect, provided herein are compositions comprising a virion library made by a method described herein (e.g., made by a method described in §§ 5.14.1, 5.14.2). In some embodiments, the virion library is made by a method described in made by a method described in § 5.14.2. In some embodiments, the virion library is made by a method described in made by a method described in § 5.14.1.

### 5.13 Kits

In a one aspect, provided herein are kits comprising any one or more of a transfer polynucleotide (e.g., transfer plasmid) described herein (see, e.g., § 5.2); a landing pad polynucleotide (e.g., landing pad plasmid) described herein (see, e.g., § 5.4); a cell (or population of cells) comprising a landing pad plasmid integrated into the cell's genome described herein (see, e.g., § 5.5); a library of transfer polynucleotides (e.g., transfer plasmids) described herein (see, e.g., § 5.3); a cell library described herein (see, e.g., § 5.6); a cell library made by a method described herein (see, e.g., § 5.14.1); a library of virions expressing and encoding a protein of interest (e.g., a viral entry protein) described herein (see, e.g., § 5.7); a virion library made by a method described herein (see, e.g., § 5.14.2); and/or a system described herein (see, e.g., § 5.11); or any combination of the foregoing.

In one embodiment, the kit comprises a transfer polynucleotide (e.g., transfer plasmid) described herein. In one embodiment, the kit comprises a landing pad polynucleotide (e.g., landing pad plasmid) described herein (see, e.g., § 5.2). In one embodiment, the kit comprises a cell (or population of cells) comprising a landing pad plasmid integrated into the cell's genome described herein. In one embodiment, the kit comprises a library of transfer polynucleotides (e.g., transfer plasmids) described herein. In one embodiment, the kit comprises a cell library encoding proteins or interest (e.g., viral entry proteins) described herein. In one embodiment, the kit comprises a library of virions expressing and encoding a protein of interest (e.g., a viral entry protein) described herein.

In some embodiments, the kit comprises instructions for use of any one or more component of the kit.

In some embodiments, the kit comprises one or more additional reagents useful to utilize any one or more of the components of the kit (e.g., to solubilize, dilute, detect, etc.).

Any of the kits described herein may be used in any of the methods described herein (see, e.g., § 5.14).

### 5.14 Methods

Provided herein are various methods of utilizing or making any one or more of a transfer polynucleotide (e.g., transfer plasmid) described herein (see, e.g., § 5.2); a landing pad polynucleotide (e.g., landing pad plasmid) described herein (see, e.g., § 5.4); a cell (or population of cells) comprising a landing pad plasmid integrated into the cell's genome described herein (see, e.g., § 5.5); a library of transfer polynucleotides (e.g., transfer plasmids) described herein (see, e.g., § 5.3); a cell library described herein (see, e.g., § 5.6); a cell library made by a method described herein (see, e.g., § 5.14.1); a library of virions expressing and encoding a protein of interest (e.g., a viral entry protein) described herein (see, e.g., § 5.7); a virion library made by a method described herein (see, e.g., § 5.14.2); and/or a system described herein (see, e.g., § 5.11); or any combination of the foregoing.

### 5.14.1 Methods of Making Cell Libraries (e.g., Encoding Viral Entry Proteins)

In one aspect, provided herein are, inter alia, methods of making cell libraries (e.g., collections) comprising a plurality of cells each encoding a protein of interest (e.g., a viral entry protein) (e.g., cell libraries described herein, see, e.g., § 5.6). Each of the cells in the library comprises an integrated landing pad polynucleotide described herein, and an integrated transfer polynucleotide described herein encoding a protein of interest (e.g., a viral entry protein).

The methods generally comprise the steps of: (a) providing a population of cells comprising a landing pad described herein that has been integrated into genomic DNA of the cells (e.g., cells described in § 5.5), (b) introducing a plurality of transfer polynucleotides (e.g., transfer plasmids) described herein (e.g., a library of transfer polynucleotide (e.g., transfer plasmids) described herein (see, e.g., § 5.3)) into the cells, (c) integrating the transfer polynucleotides into the landing pad in the cells using a recombinase that recognizes recombinase recognition sites in the landing pad and transfer polynucleotides, wherein integration of a transfer polynucleotide into the landing pad enables transcription of: (i) the polynucleotide encoding the protein of interest (e.g., the viral entry protein) under the control of a promoter sequence operably linked to the recombinase recognition site, and optionally (ii) the one or more selectable marker genes; and optionally (d) selecting cells that comprise an integrated transfer polynucleotide by detecting expression of the one or more selectable marker genes in the cells, to thereby obtain a cell library.

Transfer polynucleotides described herein can be introduced into cells comprising an integrated recombinase landing pad using standard reagents and techniques for introducing polynucleotides into cells, for example, by electroporation, lipofection, gene gun, hydroporation, magnetofection, microinjection, photoporation, sonoporation or ultrasound. In some embodiments, a transfer polynucleotide of the disclosure is introduced into cells comprising an integrated recombinase landing pad by a chemical method, for example, via dendrimers, exosomes, lipid nanoparticles lipofection, lipoplexes, liposomes, polymers, polyplexes, solid lipid nanoparticles, synthetic nanoparticles or vesicles. In certain embodiments, a transfer polynucleotide of the disclosure is introduced into cells comprising an integrated recombinase landing pad by transfection.

In some embodiments, the methods described herein comprise integrating transfer polynucleotides of the disclosure into the recombinase landing pads in the cells using a recombinase (e.g., an exogenous recombinase) that recognizes recombinase recognition sites in the recombinase landing pad and transfer polynucleotide. The recombinase protein, or a nucleic acid encoding the recombinase, can be introduced (e.g., transfected) into the cells prior to, concurrently with, or subsequent to introduction (e.g., transfection) of the transfer polynucleotides into the cells. In certain embodiments, the recombinase landing pad comprises a polynucleotide sequence encoding the recombinase. The recombinase landing pad can further comprise a promoter that is operably linked to a polynucleotide sequence encoding the recombinase. In some embodiments, the promoter that is operably linked to a polynucleotide sequence encoding a recombinase is a constitutive promoter (e.g., a CMV promoter).

The recombinase can be any of the recombinases known in the art and/or described herein including, for example, a tyrosine site-specific recombinase or a serine site-specific recombinase. In particular embodiments, the recombinase is a Bxb1 recombinase, and the polynucleotides and landing pad each comprise a recombinase recognition site that is recognized by a Bxb1 recombinase, such as an attB, attP, attP-GT, attP-GA, attB-GT, or attB-GA site.

Cells in which a transfer polynucleotide of the disclosure has been integrated into a landing pad can be selected using one or more of the selectable markers in the recombined product. For example, cells expressing a positive selection marker, such as a detectable protein (e.g., GFP) or antibiotic resistance gene, can be obtained (e.g., sorted via FACS and/or grown on selective medium containing antibiotic). In addition, or alternatively, cells in which there has been no integration of a transfer polynucleotide of the disclosure into a landing pad can be selected against, for example, based on lack of antibiotic resistance, or by activating a suicide gene that results in cell death.

The methods disclosed herein are useful, inter alia, for making libraries of cells encoding different viral entry proteins. For example, a library of transfer polynucleotides (e.g., transfer plasmids) of the disclosure can be introduced into cells that comprise an integrated recombinase landing pad, wherein each transfer polynucleotide of the library encodes a different viral entry protein. In some embodiments, each different viral entry protein comprises a unique barcode sequence, such that the barcode sequence serves as a unique identifier of the particular viral entry protein. In some embodiments, a plurality of transfer polynucleotide in the library each encodes a different variant of a reference viral entry protein; and optionally the library comprises a transfer polynucleotide encoding the reference viral entry protein. In some embodiments, each different viral entry protein comprises a unique barcode sequence, such that the barcode sequence serves as a unique identifier of the particular viral entry protein. In some embodiments, the cells comprising the integrated recombinase landing pad are cells described herein, see, e.g., § 5.5.

In some embodiments, the method further comprises expressing the proteins of interest (e.g., viral entry proteins) in the cells, for example, by activating an inducible promoter that is operably linked to a polynucleotide sequence encoding the protein of interest (e.g., viral entry protein). Once expressed, the protein of interest (e.g., viral entry protein) can be obtained and studied (e.g., characterized), for example, by isolating (e.g., purifying) the protein of interest (e.g., viral entry protein) from the cells or extracts thereof. Alternatively, or in addition, the protein of interest (e.g., viral entry protein) can be packaged into viral particles that are produced in the cells, and the resulting viral particles can be recovered from the cells and subsequently characterized.

Accordingly, in some embodiments, the methods of the disclosure further comprise the transfecting the selected cells with helper plasmids encoding one or more proteins that enable formation of virus particles that express a protein of interest (e.g., viral entry protein). In some embodiments, the helper plasmids encode one or more HIV-1 proteins selected from Tat, Gag-Pol, and Rev. In some embodiments, the methods further comprising recovering (e.g., obtaining, isolating, purifying) virus particles that express the protein of interest (e.g., viral entry protein) from the cells.

In some embodiments, a recovered (e.g., isolated protein of interest (e.g., viral entry protein)), or a virus expressing the protein of interest (e.g., viral entry protein), is subjected to one or more assay to determine one or more structural (e.g., sequence) or functional characteristic of the protein of interest (e.g., viral entry protein).

In some embodiments, assay, to determine whether the protein of interest (e.g., viral entry protein) has an activity (e.g., binding activity (e.g., to cells or receptors expressed on cells), infectivity) against a target (e.g., human cells). In some embodiments, the barcode or the protein of interest is sequenced. In some embodiments, the protein of interest is utilized in a high-throughput assay, such as a deep mutational scanning (DMS) high-throughput. In some embodiments, the protein of interest or virion is utilized in e.g., yeast surface displayed protein libraries that are subjected to Tite-Seq assays (PMID: 28035901, 32841599), Fluorescence-Activated Cell Sorting (FACS) and sequencing (PMID: 33259788), Magnetic-Activated Cell Sorting (MACS), sequencing, a virus-based assay (e.g., as described in US 2021/0147832 A1, the contents of which are incorporated herein by reference), where the protein variants in individual virions are subjected to growth or selective conditions (e.g., antibody or drug selection) in cell culture.

### 5.14.2 Methods of Making Virion Libraries

Provided herein are, inter alia, methods of making a library (e.g., collection) of virions comprising a plurality of virions, wherein each virion of the plurality expresses (on the surface) (and encodes (i.e., genotype-phenotype linked)) a different viral entry protein (e.g., a viral entry protein described herein). See, e.g., FIG. 3. The methods general comprise, (a) making or obtaining a cell library encoding different viral entry proteins described herein (see, e.g., § 5.6); (b) transfecting the library of cells of (a) with one or more helper plasmids encoding one or more viral proteins sufficient for virion production; (c) culturing the cells under conditions and for sufficient time to allow for virion production; and (d) optionally recovering (e.g., isolating, purifying, and/or quantifying) the produced virions.

In some embodiments, the virions are retrovirus based (e.g., lentiviruses (e.g., HIV)), adenovirus based, parvovirus (e.g., adeno-associated virus) based, or virus based of family orthoherpesviridae viruses (e.g., herpes viruses, e.g., herpes simplex virus). In some embodiments, the virions are retrovirus based. In some embodiments, the virions are lentivirus based. In some embodiments, the virions are HIV based. In some embodiments, the virions are adenovirus based. In some embodiments, the virions are parvovirus based. In some embodiments, the virions are adeno-associated virus based. In some embodiments, the virions are orthoherpesviridae family based. In some embodiments, the virions are herpes virus based. In some embodiments, the virions are herpes simplex virus based.

In some embodiments, the virions are replication incompetent. In some embodiments, the virions do not express or encode virulence factors (e.g., in the case of HIV-VPU, Vif, Nef).

Various viruses can be utilized for the pseudotyping backbone and are known in the art, including, e.g., HIV, MLV, and VSV-G. In some embodiments, the helper plasmids are HIV based comprise one or more plasmids encoding the HIV gag, pol, RRE, and/or rev protein.

A person of ordinary skill in the art would be able to determine and optimize suitable cell culture conditions for virion production utilizing standard known methods in the art. Likewise, methods of recovering (e.g., isolating, purifying, and quantifying) the produced virions are standard and known in the art.

### 5.14.3 Methods of Utilizing Virion Libraries

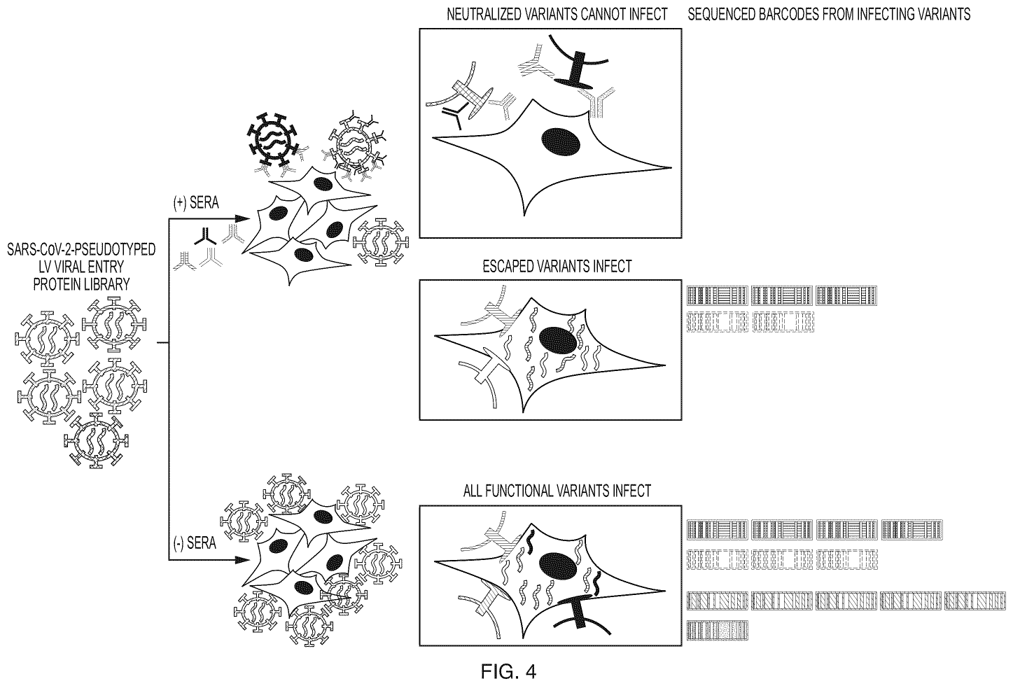

Also provided herein are various method of utilizing the virion libraries described herein (see, e.g., §§ 5.14.2, 5.7). The libraries can be used in various methods of functionally assessing the viral entry proteins (e.g., compared to each other, compared to the reference viral entry protein, etc.). Exemplary methods include, e.g., methods of assessing (determining) the ability of one or more agents (e.g., antibodies (e.g., isolated antibodies, antibodies in sera, antibodies in plasma, etc.)) to neutralize a plurality of viral entry protein. See, e.g., FIG. 4.

As such, provided herein are methods of assessing (determining) the ability of one or more agent (e.g., antibodies (e.g., isolated antibodies, antibodies in sera, antibodies in plasma, etc.)) to neutralize a plurality of viral entry proteins, the method comprising (a) making or obtaining the library of virions expressing and encoding viral entry proteins described herein (see, e.g., §§ 5.14.2, 5.7); (b) culturing a population of cells (e.g., a single population of cells) in the presence of the virion library of (a) and one or more agent (e.g., antibody) under conditions and for sufficient time to allow for infection of the cells; and (c) making a determination of whether the one or more agent (e.g., antibody) is capable of neutralizing a viral entry protein expressed by a virion of the library based on the ability of the virion within the library to infect the cells; wherein the one or more agent (e.g., antibody) is capable of neutralizing the viral entry protein if the virion does not infect the cells (or infection of the cells by the virion is not detectable).

In I some embodiments, each of the different viral entry proteins (e.g., each different variant, reference, etc.) comprises a different (unique) barcode (e.g., as described herein) (e.g., relative to each other). In some embodiments, the identity of the viral entry proteins that were not neutralized is determined by sequencing of the barcodes of the viral entry proteins inside the cultured cells.

In some embodiments, a control culture is included, wherein the control culture does not include the addition of the one or more antibodies. In such embodiments, (c) may comprise determining the ratio of barcode present in the no sera (or no monoclonal antibody) control and the sera experimental group can be compared to identify relevant escape variations in the viral entry protein variants (as compared to the reference viral entry protein).

In some embodiments, the agent is a protein, small molecule, nanoparticle (e.g., lipid nanoparticle, polynucleotide (e.g., an mRNA), a vector, or a virus.

In some embodiments, the one or more agent is one or more antibody.

In some embodiments, the one or more antibody is present in a blood sample (e.g., whole blood, sera, plasma) from a subject (e.g., a human subject, a non-human mammal subject (e.g., a ferret, mouse, hamster, non-human primate)) (or pooled blood sample (e.g., whole blood, sera, plasma) from one or more subjects (e.g., human subjects, non-human subjects)), wherein the blood sample (e.g., whole blood, sera, plasma) is added to the cell culture.

In some embodiments, the one or more antibody is present in a blood sample (e.g., whole blood, sera, plasma) from a human, wherein the blood sample (e.g., whole blood, sera, plasma) is added to the cell culture. In some embodiments, the one or more antibody is present in pooled a blood sample (e.g., whole blood, sera, plasma) from a plurality of humans, wherein the blood sample (e.g., whole blood, sera, plasma) is added to the cell culture.

In some embodiments, the one or more antibody is present in a blood sample (e.g., whole blood, sera, plasma) from a non-human mammal subject (e.g., a ferret, mouse, hamster, non-human primate) subject, wherein the blood sample (e.g., whole blood, sera, plasma) is added to the cell culture. In some embodiments, the one or more antibody is present in pooled a blood sample (e.g., whole blood, sera, plasma) from a plurality of non-human mammal subjects (e.g., a plurality of ferrets, mice, hamsters, non-human primates), wherein the blood sample (e.g., whole blood, sera, plasma) is added to the cell culture. Exemplary non-human mammals include, but are not limited to, ferrets, mice, rats, rabbits, hamsters (e.g., golden hamsters), non-human primates (e.g., rhesus macaques, long-tailed (also known as crab-cating or cynomolgus) macaques, stump-tailed macaques, pig-tailed macaques, squirrel monkeys, owl monkeys, African green monkeys, marmosets, baboons, spider monkeys, capuchin monkeys, titi monkey), sheep, cattle, pig, horses, and goats.

In some embodiments, the one or more antibody is present in a blood sample (e.g., whole blood, sera, plasma) from a ferret, wherein the blood sample (e.g., whole blood, sera, plasma) is added to the cell culture. In some embodiments, the one or more antibody is present in pooled a blood sample (e.g., whole blood, sera, plasma) from a plurality of ferrets, wherein the blood sample (e.g., whole blood, sera, plasma) is added to the cell culture.

In some embodiments, the one or more antibody is present in a blood sample (e.g., whole blood, sera, plasma) from a mouse, wherein the blood sample (e.g., whole blood, sera, plasma) is added to the cell culture. In some embodiments, the one or more antibody is present in pooled a blood sample (e.g., whole blood, sera, plasma) from a plurality of mice, wherein the blood sample (e.g., whole blood, sera, plasma) is added to the cell culture.

In some embodiments, the one or more antibody is present in a blood sample (e.g., whole blood, sera, plasma) from a hamster, wherein the blood sample (e.g., whole blood, sera, plasma) is added to the cell culture. In some embodiments, the one or more antibody is present in pooled a blood sample (e.g., whole blood, sera, plasma) from a plurality of hamsters, wherein the blood sample (e.g., whole blood, sera, plasma) is added to the cell culture.

In some embodiments, the one or more antibody is present in a blood sample (e.g., whole blood, sera, plasma) from a non-human primate, wherein the blood sample (e.g., whole blood, sera, plasma) is added to the cell culture. In some embodiments, the one or more antibody is present in pooled a blood sample (e.g., whole blood, sera, plasma) from a plurality of non-human primates, wherein the blood sample (e.g., whole blood, sera, plasma) is added to the cell culture.

In some embodiments, the blood sample (e.g., whole blood, sera, plasma) is obtained from a subject (e.g., a human subject) (or a plurality of subjects (human subjects)) that are known to have been infected with the virus that corresponds to the viral entry proteins (e.g., variants) of the library. In some embodiments, the blood sample (e.g., whole blood, sera, plasma) is obtained from a subject (e.g., a human subject) (or a plurality of subjects (human subjects)) that are known to have been infected with the virus that corresponds to the viral entry proteins (e.g., variants) of the library but at the time the blood sample (e.g., whole blood, sera, plasma) is obtained from the subject (subjects) they do not have detectable infection with the virus. In some embodiments, the blood sample (e.g., whole blood, sera, plasma) is obtained from a subject (e.g., a human subject, a non-human mammal subject) (or a plurality of subjects (e.g., human subjects, non-human mammal subject)) that are known to have been vaccinated (e.g., partially, or fully vaccinated) against the virus that corresponds to the viral entry proteins (e.g., variants) of the library.

In some embodiments, the one or more antibody is present in sera from a subject (e.g., a human subject, a non-human mammal subject) (or pooled sera from one or more subjects (e.g., human subjects, non-human mammal subjects)), wherein the sera is added to the cell culture. In some embodiments, the one or more antibody is present in sera from a human subject, wherein the sera is added to the cell culture. In some embodiments, the one or more antibody is present in pooled sera from a plurality of human subjects, wherein the sera is added to the cell culture. In some embodiments, the one or more antibody is present in sera from a non-human mammal, wherein the sera is added to the cell culture. In some embodiments, the one or more antibody is present in pooled sera from a plurality of non-human mammals, wherein the sera is added to the cell culture.

In some embodiments, the sera is obtained from a subject (e.g., a human subject, a non-human mammal subject) (or a plurality of subjects (human subjects, non-human mammal subjects)) that are known to have been infected with the virus that corresponds to the viral entry proteins (e.g., variants) of the library. In some embodiments, the sera is obtained from a subject (e.g., a human subject, a non-human mammal subject) (or a plurality of subjects (human subjects, non-human mammal subjects)) that are known to have been infected with the virus that corresponds to the viral entry proteins (e.g., variants) of the library but at the time the sera is obtained from the subject (subjects) they do not have detectable infection with the virus. In some embodiments, the sera is obtained from a subject (e.g., a human subject, a non-human mammal subject) (or a plurality of subjects (human subjects, non-human mammal subjects)) that are known to have been vaccinated (e.g., partially, or fully vaccinated) against the virus that corresponds to the viral entry proteins (e.g., variants) of the library.

In some embodiments, the one or more antibody is present in plasma from a subject (e.g., a human subject, a non-human mammal subject) (or pooled plasma from one or more subjects (e.g., human subjects, non-human mammal subjects)), wherein the plasma is added to the cell culture. In some embodiments, the one or more antibody is present in plasma from a human subject, wherein the plasma is added to the cell culture. In some embodiments, the one or more antibody is present in pooled plasma from a plurality of human subjects, wherein the plasma is added to the cell culture. In some embodiments, the one or more antibody is present in plasma from a non-human mammal subject, wherein the plasma is added to the cell culture. In some embodiments, the one or more antibody is present in pooled plasma from a plurality of non-human mammal subjects, wherein the plasma is added to the cell culture.

In some embodiments, the plasma is obtained from a subject (e.g., a human subject, a non-human mammal subject) (or a plurality of subjects (human subjects, non-human mammal subjects)) that are known to have been infected with the virus that corresponds to the viral entry proteins (e.g., variants) of the library. In some embodiments, the plasma is obtained from a subject (e.g., a human subject, a non-human mammal subject) (or a plurality of subjects (human subjects, non-human mammal subjects)) that are known to have been infected with the virus that corresponds to the viral entry proteins (e.g., variants) of the library but at the time the plasma is obtained from the subject (subjects) they do not have detectable infection with the virus. In some embodiments, the plasma is obtained from a subject (e.g., a human subject, a non-human mammal subject) (or a plurality of subjects (human subjects, non-human mammal subjects)) that are known to have been vaccinated (e.g., partially, or fully vaccinated) against the virus that corresponds to the viral entry proteins (e.g., variants) of the library.

In some embodiments, the one or more antibody is a monoclonal antibody. In some embodiments, the one or more antibody is purified and isolated. In some embodiments, the one or more antibody is a prophylactic or therapeutic antibody. In some embodiments, the one or more antibody is a prophylactic or therapeutic antibody. In some embodiments, the one or more antibody is a prophylactic or therapeutic antibody approved by a regulatory agency for use in humans (e.g., for use in the prevention, amelioration, and/or treatment of an infection with the virus that corresponds to the viral entry proteins (e.g., variants) of the library.

### 6. EXAMPLES

### Table of Contents

6.1 Example 1. Generation of an activatable cell library encoding viral entry proteins using a recombinase system.
6.2 Example 2. Landing Pad and Transfer Plasmid Design and Synthesis.
6.3 Example 3. Generation of a Genetically Engineered Cell Population Comprising Integrated Landing Pad and Barcoded Viral Entry Protein.

### 6.1 Example 1. Generation of an Activatable Cell Library Encoding Viral Entry Proteins Using a Recombinase System

The following example describes the generation of a cell library encoding viral entry proteins.

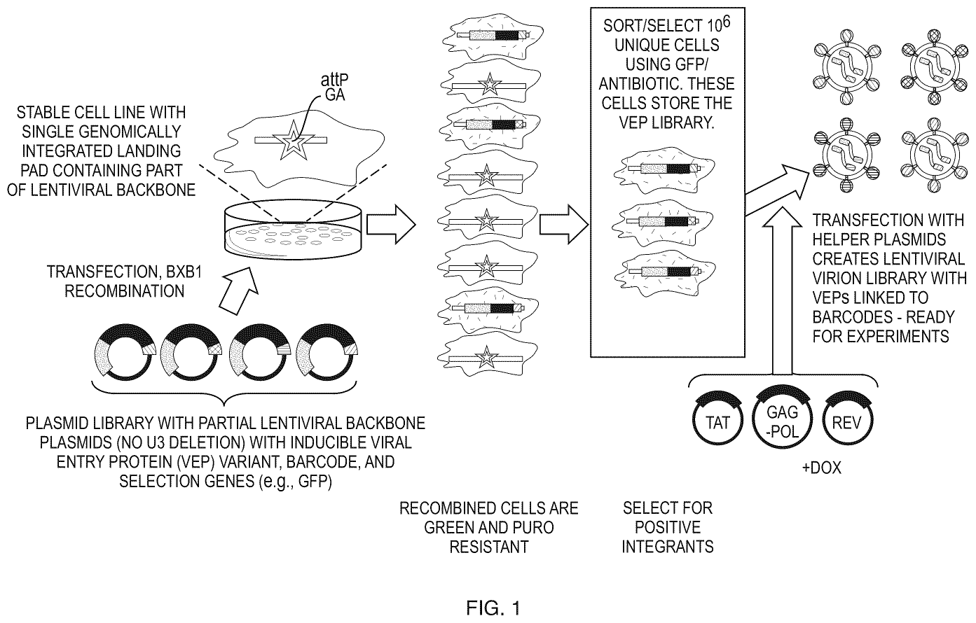

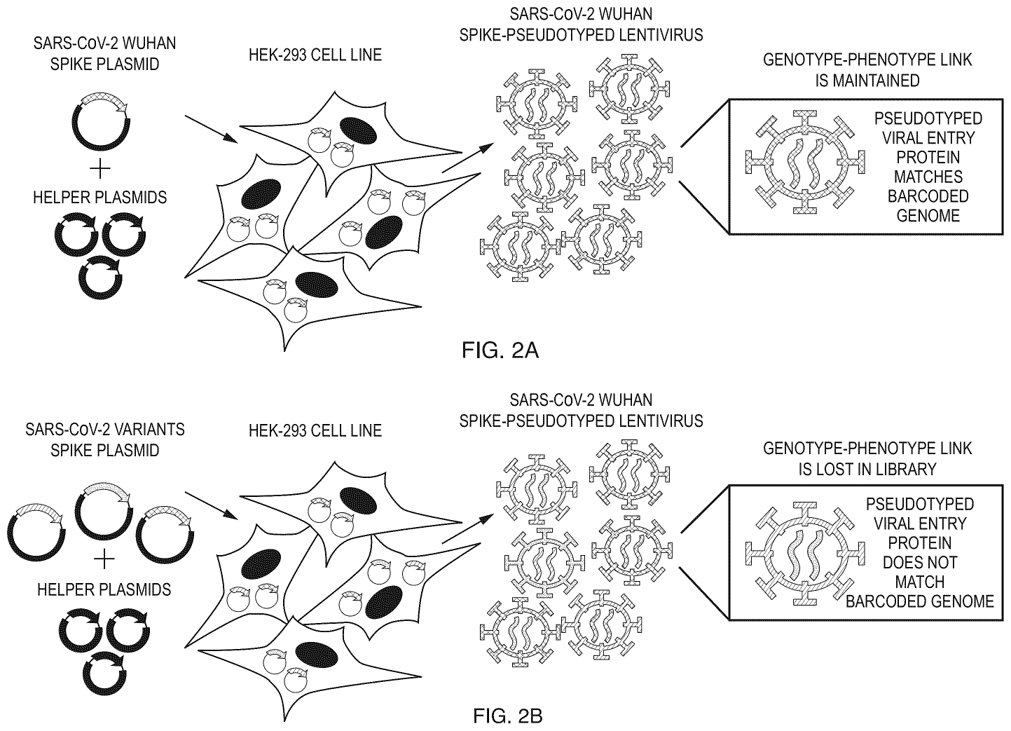

A viral entry protein (e.g., or variant thereof), barcode, and selectable marker are introduced into a defined landing pad site using a recombinase. In this Example, Bxb1 recombinase is used to deliver to an integrated attP landing pad site. The starting cell is engineered to have a single landing pad site (such as attP for Bxb1 mediated recombination) at a defined locus, along with other genomic components such as a portion of the lentiviral genome and a promoter to drive expression of an introduced selectable marker. As shown in FIG. 1, successful recombination results in introduction of the viral entry protein (e.g., or variant thereof) (under control of an inducible promoter) and unique barcodes within intact LTRs, which is subsequently able to be packaged into lentiviral particles. The recombination event also results in the introduction and expression of a selectable marker, to enable selection of integration positive cells.

FIG. 5 provides one example of the design of the cell's landing pad and the viral entry protein-containing transfer plasmid. The integrated landing pad (in this example, introduced at the AAVS1 locus) contains the 5′ end of the lentiviral genome and an inducible promoter, followed by an attP site and a blue fluorescent protein (BFP) coding sequence. Upon induction, a cell that does not receive a transfer plasmid payload would express BFP, providing a way to perform negative selection against cells that did not successfully receive a payload. There is also a selectable marker in the landing pad to retain the landing pad during cell growth before recombination. Additionally, in this design, the Bxb1 and rtTA (to enable use of the inducible promoter) genes are also integrated, but either of these can be integrated elsewhere or delivered via co-transfection.

The transfer plasmid contains an attB site, a viral entry protein (e.g., or variant thereof), barcode, and 3′ end of the lentiviral genome. In the reverse orientation on the other side of the attB site, there is an IRES followed by selectable markers (in this case, ZsGreen and Puromycin, linked by a T2A linker). Upon Bxb1-mediated recombination of the transfer plasmid into the landing pad (recombination between attB and attP shown in a grey X), the recombined product contains the entire activatable, packageable lentiviral genome and the viral entry protein driven by an inducible promoter. Additionally, the selectable markers from the transfer plasmid (here, ZsGreen-T2A-PuroR) are driven off a landing pad promoter in combination with the IRES, enabling selection for cells that successfully received an integrated transfer plasmid.

FIG. 6 provides an alternative example design of the cell's landing pad and the viral entry protein-containing transfer plasmid. In this alternative example, most of viral backbone is in the landing pad (including both the 5′ and 3′ LTRs) and the transfer plasmid lacks a partial viral genome.

The resulting cell-stored library of barcoded viral entry protein libraries of these recombinase-mediated approaches can provide, inter alia, one or more of the following advantages: high recombination rate after transfecting (e.g., 5-50%) which is more efficient relative to using low multiplicity of infection lentiviral transduction; presence of one barcoded viral entry protein (e.g., or variant thereof) per cell, enabling good control of genotype-phenotype linkage when there is a single landing pad per cell; and homogeneity in the viral entry protein pseudo-typed lentiviral library as the lentiviral genome is inserted at a defined integration site.

### 6.2 Example 2. Landing Pad and Transfer Plasmid Design and Synthesis

The following example described the design and synthesis of exemplary landing pad and transfer plasmids, along with the resulting recombined products within the genome of a cell.

The plasmid backbone of the exemplary landing pad and transfer plasmids contained an origin of replication and an ampicillin resistance cassette for replication and maintenance in E. coli, along with an SV40 origin of replication for replication in mammalian cell lines (e.g., HEK293T cells).

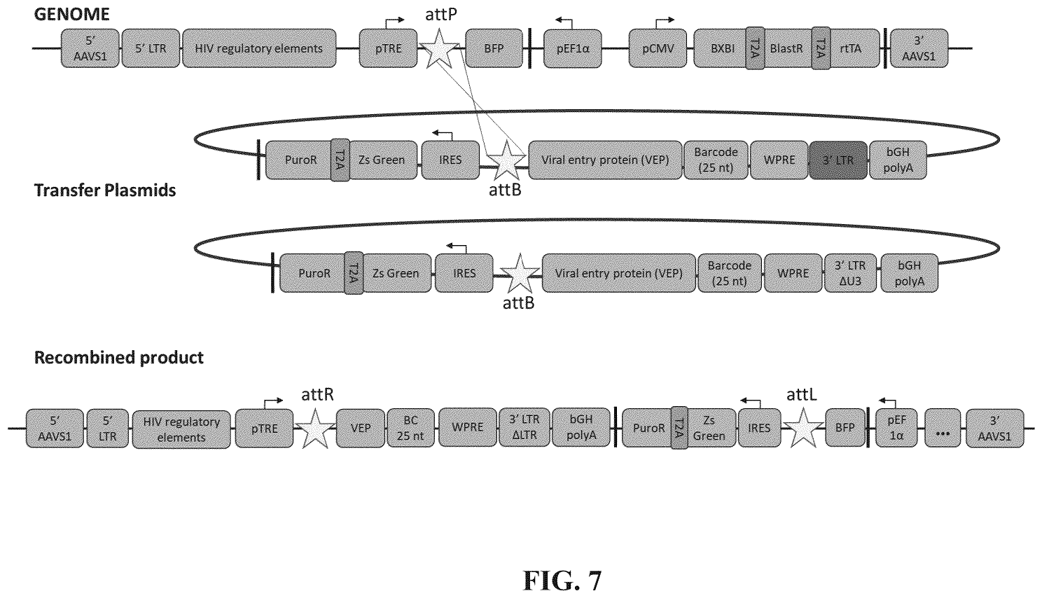

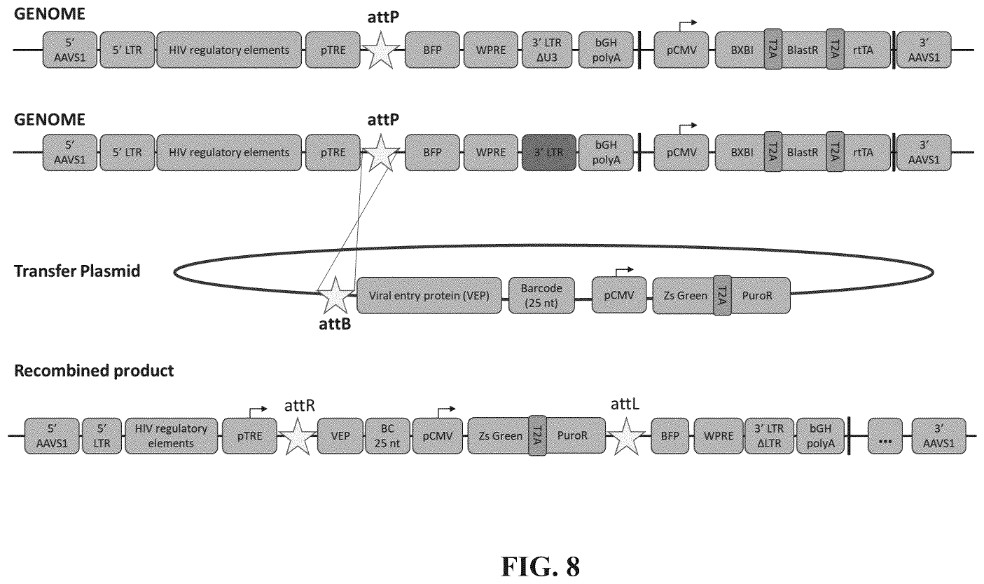

FIGS. 7-8 provide general schematics of the exemplary landing pad and transfer plasmids generated, along with the resulting recombined products. The components are described below in Table 4. It is clear to a person of ordinary skill in the art that the generated plasmids are exemplary and specified components could be removed (e.g., depending on the application), added, or swapped.

TABLE 4
Exemplary Components of Landing Pad and Transfer Plasmids.

| Component | Description |
|---|---|
| 5′ and 3′ AAVS1 | Homology arms function for targeted genomic integration of the landing pad |
| Homology Arms | plasmid using, e.g., a CRISPR based recombination approach. |
| Bxb1 Integrase | BXBI integrase recognizes specific attP and attB sequences and mediates |
| | recombination between these DNA constructs. In systems described herein the |
| | integrase mediates the integration of the transfer plasmid encoding the VEP into |
| | the integrated landing pad. |
| T2A | T2A sequences a class of 2A peptides that are amino acid sequences that mediate |
| | ribosome skipping resulting in the generation of separate polypeptides from one |
| | coding RNA (e.g., mRNA). In systems described herein the 2A peptides (e.g., |
| | T2A) function to drive the expression of multiple proteins from the same coding |
| | RNA (e.g., mRNA) under control of the same promoter. |
| pTRE, Tet | Tetracycline promoter that functions to selectively control VEP expression through |
| Promoter | the use of doxycycline. |
| rtTA Activator | Activator of the Tet-On system. The Tet-On system is based on a reverse |
| | tetracycline-controlled transactivator, rtTA. In the Tet-On system, transcription of |
| | the TRE-regulated VEP is stimulated by rtTA only in the presence of doxycycline. |
| PolyA Signal | PolyA sequence that mediates mRNA polyadenylation. |
| WPRE | Woodchuck hepatitis virus post-transcriptional regulatory element (WPRE) |
| | mediates enhanced transcription and protein expression. |
| BFP | Blue fluorescence protein utilized as a drop out marker to assess successful |
| | integration of a transfer plasmid into a landing pad. Initially BFP is under control |
| | of the Tet promoter and is replaced with the VEP after successful integration of the |
| | transfer plasmid. |
| BXBI attP | attP is the recognition sequence for BXBI integrase located in the landing pad. |
| BXBI attB | attB is the recognition sequence for BXBI integrase located in the transfer plasmid. |
| VEP | Viral entry protein of any enveloped virus. |
| CMV Promoter | Constitutive CMV promoter that drives expression of the selective markers, BXBI |
| | integrase, and the transcriptional regulator rtTA. |
| Zsgreen | Fluorescence marker that is utilized to assess the presence of the transfer plasmid |
| | in the target cells. Together with the BFP from the landing pad this marker can be |
| | used to select out cells that successfully integrated the transfer plasmid (e.g., |
| | selection of cells that are BFP negative and Zsgreen positive). |
| Puromycin | Selection marker that is utilized to select for cells that contain the transfer plasmid. |
| Resistance Gene | |
| (PuroR) | |
| Blasticidin | Resistance marker that is utilized to select for the presence of the landing pad in a |
| Resistance Gene | newly generated cell line. |
| (BlastR) | |

Viral Genome Components

| | |
|---|---|
| 3′ LTR | Provides signals for the termination and polyadenylation of the viral transcripts. |
| 3′ LTR_ΔU3 | 3′ LTR with U3 region deleted that renders the virus “self-inactivating” (SIN) after |
| | integration into the target cell genome. |
| HIV cpPu | HIV central polypurine tract that is the recognition site for proviral DNA synthesis. |
| | The HIV cpPu functions to increase transduction efficiency and transgene (VEP) |
| | expression. |
| RRE | Segment in the viral RNA that serves as a binding site for the Rev protein. The |
| | primary function of the RRE-Rev interaction is to regulate the transport of viral |
| | RNA from the nucleus (where it is generated) to the cytoplasm (where it is used to |
| | generate viral proteins). |
| PSI (Ψ) | Mediates viral particle packaging. It is recognized by the viral protein Gag, and it |
| | is this interaction that leads to the packaging of the viral genome into the |
| | assembling virus particle. |
| 5′ LTR | 5′ LTR contains elements necessary for the initiation transcription such as the |
| | TATA box and various binding sites for transcription factors. Functions as a |
| | promoter and enhancer for viral gene transcription |

### 6.3 Example 3. Generation of a Genetically Engineered Cell Population Comprising Integrated Landing Pad and Barcoded Viral Entry Protein

As described above in Example 1, exemplary systems described herein utilize a landing pad plasmid (pLP) and a series of transfer plasmids (pTF) (each encoding a viral entry protein (VEP) (or variant thereof)) to produce an in vitro cell library encoding viral entry proteins.

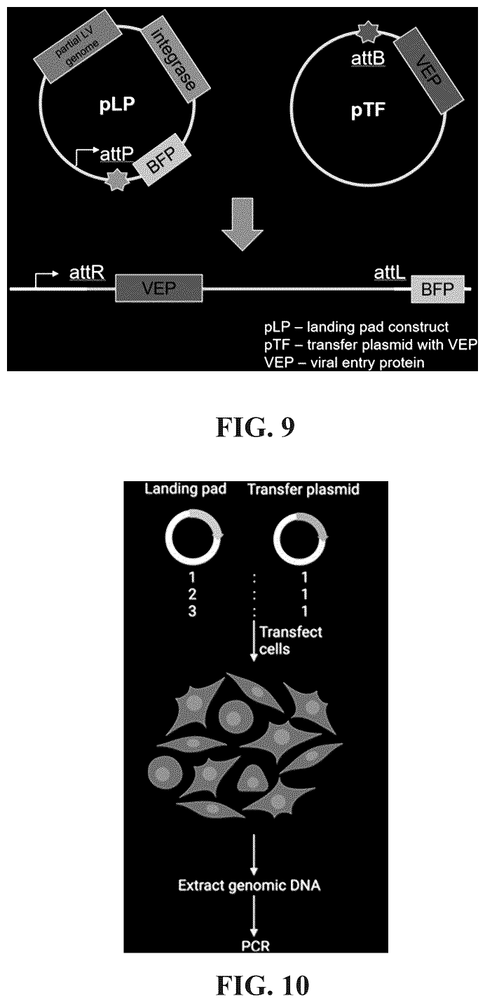

FIG. 9 provides a schematic overview of an exemplary landing pad system and recombined product post integration. The following example describes the generation of a cell line containing the integrated recombined product (produced from integration of the landing pad in the cellular genomic DNA and subsequent integration of the transfer plasmid into the landing pad) (FIG. 10).

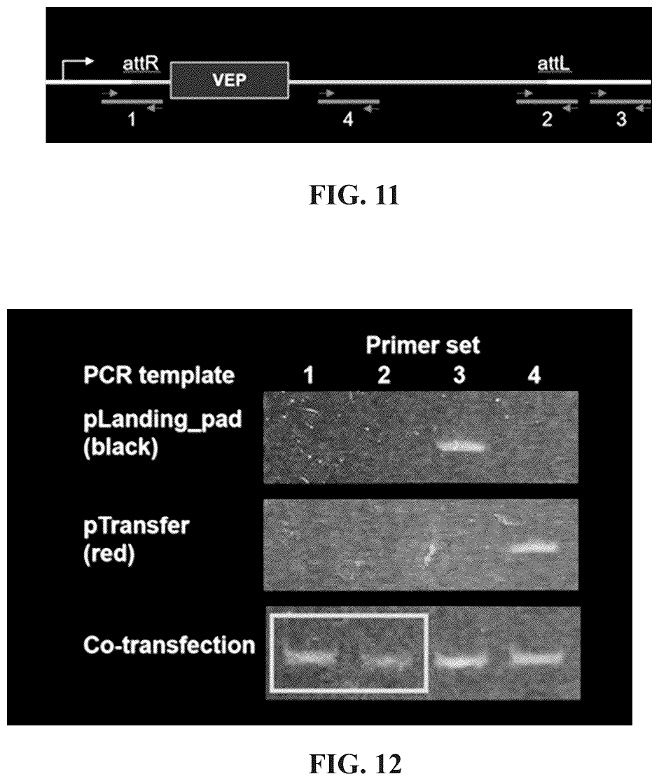

HEK-293T cells cultured in Dulbecco's Modified Eagle Medium containing 10% fetal bovine serum were co-transfected with the landing pad plasmid and the transfer plasmid (corresponding to FIG. 8) at a total concentration of 4 μg per million cells, 48 hours post transfection genomic DNA (gDNA) was isolated from the cells using standard methods using column-based purification (Qiagen Dneasy blood and tissue kit). 40-50 ng. The isolated gDNA was analyzed by polymerase chain reaction (PCR) to assess integration (integration of landing pad) and recombination (integration of the transfer plasmid into the integrated landing pad). Briefly, four sets of PCR primers were designed to amplify DNA fragments at the expected sizes using the gDNA as the template. Primer set 1 was designed to span the newly formed integration site at attR; primer set 2 was designed to span the newly formed integration site at attL, primer set 3 was designed to be specific for a portion of the landing pad plasmid; and primer set 4 was designed to be specific for a portion of the transfer plasmid (FIG. 11). The nucleotide sequence of the primers is set forth in Table 5.

TABLE 5
Nucleotide Sequence of PCR Primers.

| Primer | | SEQ | | SEQ |
|---|---|---|---|---|
| Set | Forward Primer | ID NO | Reverse Primer | ID NO |
| 1 | GCAGAGATCCAGTTTGGACTAG | 9 | CTCCAGATCCATCAAAAAAGGCT | 13 |
| | TCTCTC | | GTGA | |
| 2 | GTGAGCGAGGAAGCGGAAGAG | 10 | CTTGTCAGCCATGATGTACACAT | 14 |
| | | | TATGACTATTGAAGTTATATTC | |
| 3 | ATGGTGAGCAAAGGTGAAGAAC | 11 | ACACCACGCCACGTTGC | 15 |
| | TGTTCA | | | |
| 4 | ATGGCACAATCTAAGCATGGAT | 12 | CCGCAAAGACAGCTCCAGC | 16 |
| | TGACTAAGG | | | |
| 5 | AGGGAGTTTTCCACACGGACAC | 17 | TTCCTCCGTGCGTCAGTTTTACC | 22 |
| | CCCCCTCCTCACCACAGCCCTG | | TGTGAGATAAGGCCAGTAGCCAG | |
| | CCAGGACCACCTTATATTCCCA | | CCCCGGCCTAAGTTCAACGCGTA | |
| | ATGGAAG | | TAAGATACAT | |
| 6 | GGAACGGGGCTCAGTCTGA | 18 | CCCTTTCGCTTTCAAGTCCCTGT | 23 |
| | | | T | |
| 7 | AGTTGTGGTTTGTCCAAACTCA | 19 | GGCCCACTGTTTCCCCTTC | 24 |
| | TCAATGTATCTTAT | | | |
| 8 | GGAACGGGGCTCAGTCTGA | 20 | ACACCACGCCACGTTGC | 25 |
| 9 | ATGGTGAGCAAAGGTGAAGAAC | 21 | CTCTGGTTCTGGGTACTTTTATC | 26 |
| | TGTTCA | | TGTCC | |

PCR analysis showed the successful integration of the landing pad plasmid into the gDNA and successful integration of the transfer plasmid into the landing pad plasmid resulting in the recombined product (FIG. 12).

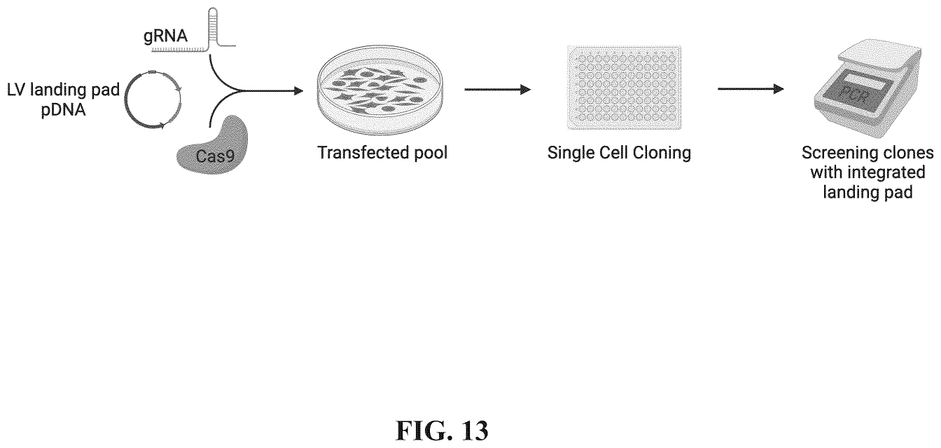

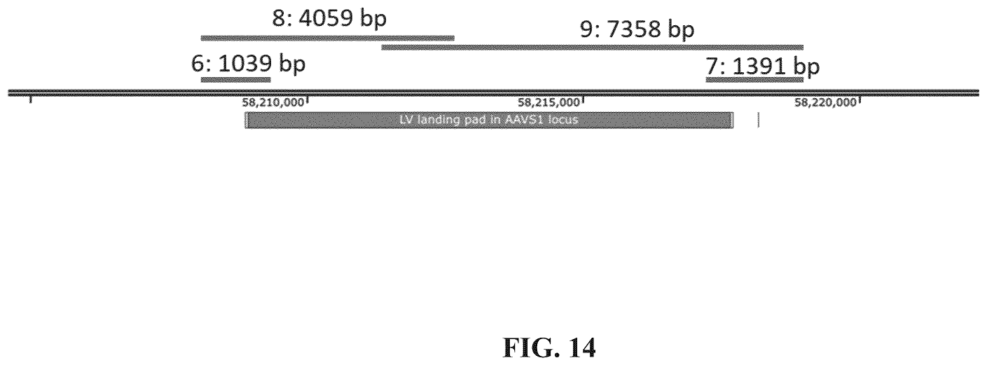

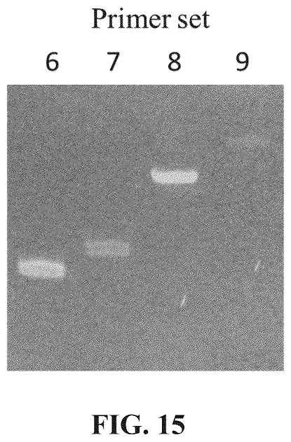

For generation of a stable HEK-293T landing pad cell line, the landing pad sequence (corresponding to FIG. 8) was amplified by PCR using primer set 5. HEK-293T cells cultured in Dulbecco's Modified Eagle Medium containing 10% fetal bovine serum were transfected with the amplified landing pad DNA at a total concentration of 1.5 ug DNA per million cells. 48 hours post transfection, cell culture media containing 10 μg/ml Blasticidin was added to the cells in order to select for successfully transfected cells. After 72 hours of Blasticidin selection, cells were detached and diluted to allow for selection of single cell clones. Cells were allowed to grow for 2-3 weeks and gDNA from single-cell-clone-derived cultures was screened to demonstrate landing pad integration using PCR primer sets 6-9 (FIGS. 13-15).

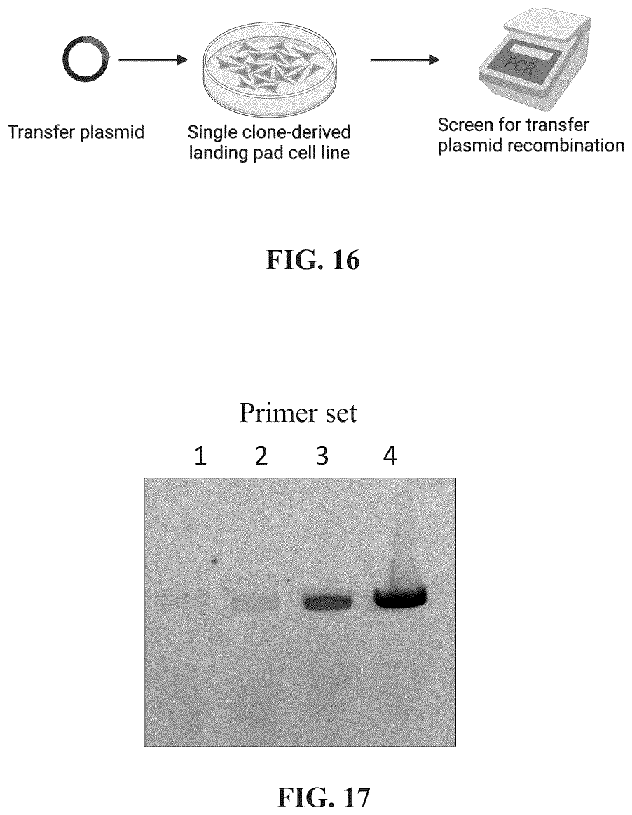

To assess recombination using the landing pad system, single-cell-clone-derived HEK-293T landing pad cells were transfected with the transfer plasmid DNA (corresponding to FIG. 8) at a total concentration of 4 μg per million cells. 72 hours post transfection, isolated gDNA was analyzed by PCR using primer sets 1-4 to assess recombination (integration of the transfer plasmid into the integrated landing pad) (FIG. 16 and FIG. 17)

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A transfer polynucleotide comprising: a polynucleotide sequence encoding a protein of interest, one or more selectable marker genes, and a recombinase recognition site, wherein the transfer polynucleotide is transcriptionally inactive.

2.-11. (canceled)

12. The transfer polynucleotide of claim 1, wherein the protein of interest is a viral entry protein (or a variant or fragment thereof).

13.-27. (canceled)

28. A library of transfer polynucleotides comprising a plurality of transfer polynucleotides of claim 1.

29.-31. (canceled)

32. A landing pad polynucleotide comprising: a partial viral genome, a recombinase recognition site, and a promoter operably linked to the recombinase recognition site.

33.-57. (canceled)

58. A cell comprising the landing pad polynucleotide of claim 32 integrated into the genomic DNA of the cell.

59.-63. (canceled)

64. A library of cells comprising a plurality of cells of claim 58 and each cell further comprises a transfer polynucleotide integrated into the integrated landing pad.

65.-67. (canceled)

68. A vector comprising the transfer polynucleotide of claim 1.

69. A vector comprising the landing pad polynucleotide of claim 32.

70.-71. (canceled)

72. A cell (or population of cells) comprising the transfer polynucleotide of claim 1 or a library thereof.

73. A system comprising (i) the transfer polynucleotide of claim 1; and (ii) a landing pad polynucleotide comprising: a partial viral genome, a recombinase recognition site, and a promoter operably linked to the recombinase recognition site.

74. A system comprising (i) the transfer polynucleotide of claim 1; and (ii) a cell comprising a landing pad polynucleotide comprising: a partial viral genome, a recombinase recognition site, and a promoter operably linked to the recombinase recognition site integrated into the genomic DNA of the cell.

75. A system comprising (i) the library of transfer polynucleotides of claim 28; and (ii) a cell comprising a landing pad polynucleotide comprising: a partial viral genome, a recombinase recognition site, and a promoter operably linked to the recombinase recognition site integrated into the genomic DNA of the cell.

76. A system comprising (i) the cell library of claim 64; and (ii) one or more helper plasmids encoding one or more viral proteins sufficient for virion production in combination with the library.

77.-78. (canceled)

79. A composition comprising transfer polynucleotide of claim 1.

80. A kit comprising the transfer polynucleotide of claim 1.

81. A method of making a cell library, the method comprising:
(a) making or obtaining a plurality of cells comprising a landing pad polynucleotide comprising: a partial viral genome, a recombinase recognition site, and a promoter operably linked to the recombinase recognition site integrated into the genomic DNA of each of the cells;
(b) introducing the library of transfer polynucleotides of claim 28 into the cells;
(c) culturing the cells under conditions and for a period of time sufficient to allow for recombinase mediated integration of a transfer polynucleotide into the integrated landing pad in a cell, wherein integration of a transfer polynucleotide into the landing pad enables transcription of: (i) the polynucleotide from the transfer polynucleotide encoding the protein of interest under the control of the promoter operably linked to the recombinase recognition site from the landing pad, and (ii) the one or more selectable marker genes from the transfer polynucleotide;
(d) optionally selecting cells that comprise an integrated transfer polynucleotide by detecting expression of the one or more selectable marker genes from the transfer polynucleotide in the cells
to thereby obtain a library of cells encoding proteins of interest.

82.-95. (canceled)

96. A library of cells made by the method of claim 81.

97. The method of claim 81, further comprising transfecting the selected cells with one or more helper plasmids encoding one or more proteins viral proteins that enable formation of virions that express and encode the proteins.

98.-99. (canceled)

100. A library of virions comprising a plurality of virions made the method of claim 97.

101. A method of making a library of virions, the method comprising
(a) making or obtaining the library of cells of claim 64, wherein each cell in the library comprises integrated transfer polynucleotide that encodes a different viral entry protein;
(b) transfecting the library of cells of (a) with one or more helper plasmids encoding one or more viral proteins sufficient for virion production; and
(c) culturing the cells under conditions and for sufficient time to allow for virion production; and
(d) optionally isolating, purifying, and/or quantifying the produced virions.

102.-107. (canceled)

108. A library of virions comprising a plurality of virions made by the method of claim 101.

109. A method of assessing the ability of one or more agents (e.g., antibodies) to neutralize a plurality of different viral entry proteins, the method comprising
(a) making or obtaining the library of virions of claim 108;
(b) culturing a population of cells in the presence of the virion library of (a) and one or more agent under conditions and for sufficient time to allow for infection of the cells; and
(c) making a determination of whether the one or more agent is capable of neutralizing a viral entry protein expressed by a virion of the library based on the ability of the virion within the library to infect the cells; wherein the one or more agent is capable of neutralizing the viral entry protein if the virion does not infect the cells (or infection of the cells by the virion is not detectable).

110.-117. (canceled)

118. A system comprising (i) the cell library of claim 96; and (ii) one or more of helper plasmids encoding one or more viral proteins sufficient for virion production in combination with the library.

119. A system comprising (i) the library of virions expressing and encoding protein of claim 100; and (ii) a population of cells.

120. A system comprising (i) the library of virions expressing and encoding protein of claim 108; and (ii) a population of cells.
